(19) United States
(12) Patent Application Publication
(10) Pub. No.: US 2025/0082897 A1
(43) Pub. Date: Mar. 13, 2025

(54) CATHETER ASSEMBLY

(71) Applicant: ConvaTec Limited, Flintshire (GB)

(72) Inventor: Oliver Pfleger, Spital (GB)

(21) Appl. No.: 18/891,131

(22) Filed: Sep. 20, 2024

Related U.S. Application Data
(63) Continuation of application No. PCT/GB2023/050687, filed on Mar. 20, 2023.
(60) Provisional application No. 63/349,641, filed on Jun. 7, 2022.
(60) Provisional application No. 63/394,717, filed on Aug. 3, 2022.
(60) Provisional application No. 63/394,710, filed on Aug. 3, 2022.

(30) Foreign Application Priority Data
Mar. 21, 2022 (GB) 2203882.2
Mar. 21, 2022 (GB) GB2203893.9
Aug. 12, 2022 (GB) 2211843.4

(51) Int. Cl. A61M 25/00; A61M 39/24

(52) U.S. Cl. CPC A61M 25/0043; A61M 25/0009; A61M 25/002; A61M 39/24; A61M 2025/0062; A61M 2202/0014; A61M 2202/0496; A61M 2205/0222; A61M 2207/10; A61M 2209/06; A61M 2210/1078

(57) ABSTRACT

A catheter assembly having a catheter having a proximal end for insertion ingot the body and a distal end, a fluid collection bag arranged to receive fluid from the distal end of the catheter, a sleeve enclosing the catheter along the length of the catheter, and a fluid reservoir arranged at the proximal end. The fluid reservoir is configured to release wetting fluid into the sleeve to wet the catheter and the distal end of the sleeve is in fluid communication with the fluid collection bag. Fluid may flow from the distal end of the catheter into the sleeve or from the distal end of the sleeve into the catheter. An inlet to receive fluid from the distal ends of the sleeve and catheter. The inlet may comprise a one-way valve. There is also provided a packaged catheter assembly comprising a pouch containing the catheter assembly.

### CROSS REFERENCE TO RELATED DISCLOSURES

The present disclosure is a Continuation of International Application No. PCT/GB2023/050687 filed on Mar. 20, 2023 and claims the benefit of GB2203882.2 filed Mar. 21, 2022; GB2203893.9 filed Mar. 21, 2022; GB2211843.4 filed Aug. 12, 2022; U.S. 63/349,641 filed Jun. 7, 2022, U.S. 63/394,710 filed Aug. 3, 2022; and U.S. 63/394,717 filed Aug. 3, 2022; the disclosures of which being incorporated herein by references in entirety.

### TECHNICAL FIELD OF THE INVENTION

The present invention relates to catheter assemblies. In particular the invention concerns packaged urinary catheter assemblies, and most particularly, but not exclusively packaged intermittent male urinary catheter assemblies, especially “closed” catheter assemblies.

### BACKGROUND TO THE INVENTION

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows them to enjoy a relatively normal lifestyle.

Many existing catheter assemblies require the catheter to be strapped to a user's body, typically the user's leg to support the fluid collection bag when in use. This is conventional when the catheter is a permanent catheter, such as a Foley catheter. For intermittent catheters, that is, catheters which are used intermittently throughout a day for short term catheter-based management of the bladder, it is not practical or comfortable for the user to have a catheter strapped to their body for the entire day when the catheter is intended to be inserted into the user only long enough to drain the bladder, then removed. As such, when a user intends to use an intermittent catheter, they must either hold the fluid collection bag during use or find a suitable place to rest the bag. In each instance, this is cumbersome and creates problems for the user, in particular for users with limited dexterity. It must be appreciated that holding a fluid collection bag by the bag itself during urination is increasingly difficult when the bag is being filled with liquid. This is because the user may grip the bag too tightly, which can disrupt the filling of the bag, cause the distal end of the catheter to detach from the bag, or cause the bag to overflow with liquid. In each case, what should be a private and discrete moment for the user, can become problematic and embarrassing.

In some existing catheter assemblies, the fluid collection bag is provided with an outlet valve for releasing the contents of the bag after use. This is particularly common in permanent catheter assemblies, such as a Foley catheter. Permanent catheters are attached, for example by a strap, to the body of a user, typically the user's leg. As such, movement of the fluid collection bag is prevented when the user operates the outlet valve. Moreover, permanent catheters comprise a ‘north-south’ outlet valve, meaning that the valve must be actuated in an upward and downward motion to open and close the valve. Thus, the valve is arranged parallel to the user's leg, so the user does not accidentally open the valve when crossing their legs, as lateral movement will not open the valve. Moreover, the ‘north-south’ outlet valve on a catheter of this type is typically operable by a user who is fully dextrous (having the Foley catheter as a temporary measure) or by a healthcare professional on the user's behalf, where the user is bedridden. However, if the user has limited dexterity or the catheter assembly was not attached to the user's body, for example where the catheter is an intermittent catheter, then ‘north-south’ outlet valves would be cumbersome to operate as they require the fluid collection bag to be held whilst operating the valve. This can be problematic for the user making the outlet valves difficult to operate.

To reduce the risk of damage to the urinary tract and improve comfort while using the catheter, it is also important they are activated before use for example through wetting of the catheter by a wetting fluid. However, once wetted and/or used a catheter can be slippery and difficult to handle. This can make handling and re-packaging of the catheter after use difficult and unhygienic, as the catheter may inadvertently contact the user's hands, clothing or personal items. Existing intermittent catheters disclose continuous handling sleeves that cover the entire length of the catheter. While these provide some assistance to handling, the sleeve can make the catheter more difficult to wet as wetting fluid may not be able to easily reach all parts of the catheter.

When using a catheter, it is important that the catheter remains clean but when packaged this can be challenging as the catheter must be removed from the packaging. This can lead to the user touching a part of the catheter that is intended to be introduced into the body, such as the tube, by accident while they attempt to remove the catheter from the packaging which can lead to a risk of infection during use. It is therefore important to provide packaging which is convenient and easy for the user to open, and which presents the catheter to the user such that it can be removed from the packaging for use without accidentally touching a part of the catheter that should remain clean directly.

To provide discrete and portable catheter assemblies, packaging may be provided to contain the catheter and any other equipment such as wetting reservoirs or sachets and fluid collection bags before and/or after use. However, these items may become wet which makes handling and re-packaging of them difficult. In addition, where liquid is contained within a sachet, reservoir or bag within the packaging, any release (intentional or otherwise) of this may cause liquid to accidentally leak out onto parts of the catheter assembly that the user has to touch-such as a handling element, or onto the packaging causing problems for the user.

Users can also have problems in opening packaging and accessing its contents. This can happen because the packaging must be robust enough to ensure its contents is not inadvertently released and so opening of the packaging may require careful manipulation as well as a large force. This can mean the user either cannot use the catheter as they cannot access it or that the package is not opened in a controlled manner. This can cause embarrassment for the user but also inconvenience as the catheter may be rendered useless if it is allowed to fall on the floor or otherwise comes into contact with dirt.

When using a catheter assembly, often multiple different actions are required to prepare the catheter for use. These actions may also require the manipulation of different parts of the catheter assembly and as such at any one time one or more parts of the catheter assembly may not be required. This can make the catheter difficult to use as the user must carefully hold and manipulate the different parts of the assembly while ensuring the entire assembly remains clean and free from dirt to minimise the risk of infection.

To allow self-catheterisation as required, closed catheter assemblies collect and retain urine using a fluid collection bag, which removes the need for urine to pass directly into a toilet. To provide a fluid collection bag that is discreet prior to use but also able to store a suitable volume of liquid, fluid collection bags can be substantially 2D prior to use, consisting of two panels joined at their periphery. However, this can cause problems for the user as they must support or hold the fluid collection bag to ensure that it fills during use. Once used, a fluid collection bag is also heavy and bulky as it is filled with liquid. To allow the bag to be carried and disposed of more discretely and easily, existing bags may be provided with a drain to allow them to be emptied into a toilet. However, existing drains can either leak or are difficult to open which potentially results in inadvertent or uncontrolled release of liquid from the bag. In addition, after use the bag may be stored before cleaning or disposal, however, the drain can make storage of the bag difficult and unhygienic, as well as adding bulk to the catheter assembly which makes it less discreet and consequently less desirable for the user.

It is an object of embodiments of the present invention to at least partially overcome or alleviate the above problems and/or to provide an improved catheter assembly.

### SUMMARY OF THE INVENTION

In broad terms, the present invention concerns a catheter assembly comprising a catheter.

According to a first aspect of the present invention there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag. Consequently, excess wetting fluid may flow from the sleeve into the fluid collection bag.

Advantageously, the present invention provides for a catheter assembly which benefits from the introduction of wetting fluid at the proximal end of the catheter which reduces the risk of discomfort and injury while using the catheter as the proximal end, which enters the body first, is adequately wetted prior to use. In addition, as the wetting fluid is contained within the sleeve and flows from the proximal end to the distal end, the fluid is used efficiently to wet the whole length of the catheter by prioritising the parts of the catheter that enter the body first. Due to the sleeve being in fluid communication with the fluid collection bag, wetting fluid cannot escape outside the sleeve and as such the user's hands or clothing does not inadvertently contact the wetting fluid which could make handling the catheter difficult. This ensures that the catheter is easy and convenient to use, as well as being discrete and hygienic to dispose of as the wetting fluid is contained within the sleeve/bag.

The catheter may be a urinary catheter. The catheter may be a male urinary catheter (i.e. a catheter for catheterisation of men). The catheter may be a female urinary catheter (i.e. a catheter for catheterisation of women). The catheter may be an intermittent catheter. In one embodiment, the catheter is an intermittent male urinary catheter. Thus, the features of the present invention allow intermittent male urinary catheters to be adequately wetted prior to use which can be more difficult than for other types of catheters which are generally shorter.

The sleeve may comprise a flexible plastics material. The sleeve may be liquid impermeable. The sleeve may comprise a thermoplastic polyurethane (TPU) or low-density polyethylene (LDPE). Thus, the sleeve is cheap and easy to produce and can be easily manipulated by the user during use.

The catheter may comprise a proximal end for insertion into the body and a distal end. The sleeve may comprise a proximal end and a distal end. The distal end of the sleeve may be secured or attached either directly or indirectly to the distal end of the catheter. The sleeve may be configured to enclose a majority of the length of the catheter. The sleeve may be liquid permeable. Where the sleeve is liquid permeable, it may be provided in a configuration in which it covers a majority of the length of the catheter.

The fluid reservoir may be configured to retain wetting fluid within it. The fluid reservoir may comprise a deformable, frangible or burstable sachet. A deformable, frangible or burstable reservoir enhances the convenience for a user as fluid can be easily released without additional tools. A sachet in particular can be more easily handled on an assembly line compared to handling fluid itself.

The wetting fluid may activate the surface of the catheter and render it lubricious. The wetting fluid may be water, such as where the catheter has a hydrophilic surface. Any suitable wetting fluid may be used, for example polar (e.g. water-based) or non-polar (e.g. oil-based) wetting fluids depending on the surface properties of the catheter.

The fluid reservoir may be configured to retain at least 5, 10, 15, 25, or 50 ml of wetting fluid. The fluid reservoir may be configured to retain the wetting fluid within it. The fluid reservoir may be configured to retain at least 5, 10, 15, 25, or 50 ml of wetting fluid, most preferably at least 12 ml of fluid. The fluid reservoir may be configured to release wetting fluid into the sleeve; in particular the fluid reservoir may be configured to release wetting fluid between an internal surface of the sleeve and an external surface of the catheter. The fluid reservoir may be configured to retain and/or release excess wetting fluid. Excess wetting fluid is defined herein as more than is normally required to activate/wet the catheter, for example 10%, 20%, 50% or more, in excess of that which would normally be provided to wet the catheter (having regard for example to the length of the catheter and the composition of the wetting fluid). The sleeve may be configured to allow excess wetting fluid to flow into the fluid collection bag. Thus, the reservoir may release an excess of wetting fluid to ensure that the catheter is well wetted and excess fluid can flow into the fluid collection bag ensuring it does not negatively affect subsequent use of the catheter.

The fluid collection bag may be any suitable shape or size, for example rectangular, circular, elliptical, cuboid, spherical, etc. The fluid collection bag may comprise a front panel and a rear panel. The fluid collection bag may comprise a peripheral bond joining the periphery of the front panel and rear panel to form the fluid collection bag. The peripheral bond may define a base, two lateral edges and an upper edge of the fluid collection bag. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag. The bag may have a width between the left lateral edge and right lateral edge of at least 10, 12, 15 or 20 cm. The bag may have a width no more than 25, 20, or 15 cm. Preferably, the width is between 10 and 20 cm, most preferably between 12 and 15 cm. The base may define a bottom of the bag. The upper edge may define a top of the bag. The height of the bag from the base to the upper edge may be at least 25, 30, 35 or 40 cm. The height of the bag may be no more than 45, 40, 35 or 30 cm. The height may be between 25 and 40 cm, 30 and 35 cm, or most preferably 33 and 35 cm. The bag may be configured to hold at least 500, 700, or 1000 ml of fluid, and may hold no more than 1000 ml, for example, it may hold at least 700 ml of fluid.

The fluid collection bag may comprise a flexible plastics material. For example, polypropylene (PP), polyethylene terephthalate (PET), low density polyethylene (LDPE), metalized polyester (MET PET), orientated polypropylene (OPP), or polyvinyl chloride (PVC).

The peripheral bond may provide a water-tight seal. Preferably, the peripheral bond provides a sterile seal. The peripheral bond may comprise any one or more of: a weld; mechanical seal; heat seal; pressure seal; adhesive; solvent bond; ultraviolet bond; ultrasonic weld; laser weld; impulse weld; or friction weld. This ensures that the catheter is maintained within a sterile environment prior to use and also that once the catheter has been used any fluid contained within the bag does not leak out unintentionally.

The fluid collection bag may comprise an inlet to allow fluid to enter the bag, for example from the distal end of the catheter and/or the sleeve. The inlet may be connected to one end of the sleeve which corresponds to the distal end of the catheter. The inlet may comprise a one-way valve which prevents fluid flow through the inlet from the fluid collection bag to the sleeve and/or catheter. Thus, the inlet allows liquid to pass into the fluid collection bag but optionally may prevent fluid flow out of the bag and into the sleeve. This can reduce the likelihood of leaking.

Accordingly, in one embodiment there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag wherein the fluid collection bag comprises an inlet to allow fluid to enter the bag and the inlet comprises a one-way valve which prevents fluid flow through the inlet from the fluid collection bag to the sleeve and/or catheter.

The sleeve may comprise a distal end corresponding to the distal end of the catheter. The distal end of the sleeve may be attached to the distal end of the catheter. The inlet may be configured to allow wetting fluid to enter the fluid collection bag from the distal end of the sleeve and optionally via the catheter. Thus, excess wetting fluid can drain out of the sleeve if required.

Accordingly, in one embodiment, there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag, wherein the sleeve comprises a distal end corresponding to the distal end of the catheter and the fluid collection bag comprises an inlet configured to allow fluid to enter the fluid collection bag from the distal end of the sleeve and optionally via the catheter.

The catheter assembly may be configured to allow fluid to pass directly from the sleeve to the inlet and/or directly from the catheter to the inlet. One lateral side (e.g. a right lateral side) of the catheter may be attached to one side (e.g. a right side) of the inlet. There may be a gap between a different lateral side (e.g. a left lateral side) of the catheter and a respective different side (e.g. a left side) of the inlet. The distal end of the catheter may be within the inlet. The sleeve may be sealed around a periphery of the inlet. As such, fluid travelling within the sleeve is directed into the inlet directly via the gap. In addition, fluid travelling within the catheter passes out the distal end and into the inlet. In other examples, the catheter may be positioned differently and may be attached, for example, to the left side of the inlet.

The catheter assembly may be configured to allow fluid to pass from the catheter into the sleeve. One lateral side (e.g. a right lateral side) of the catheter may be attached to a side (e.g. right side) of the sleeve. A distal end of the catheter may be within the sleeve. A distal end of the catheter may not be within the inlet. The sleeve may be sealed around a periphery of the inlet. As such, fluid travelling within the sleeve is directed into the inlet directly and fluid travelling within the catheter passes out the distal end into the sleeve and then from there into the inlet. In other examples, the catheter may be positioned differently and may be attached to a left side of the sleeve (or indeed a front side or rear side). Furthermore, the distal end of the catheter may be blocked and an outlet may be provided in a lateral side of the catheter to allow fluid to pass out into the sleeve.

The catheter assembly may be configured to allow fluid to pass from the sleeve into the catheter. The catheter assembly may be configured to allow fluid to pass from the distal end of sleeve into the catheter. One lateral side (e.g. a right lateral side) of the catheter may be attached to one side (e.g. a right side) of the inlet. The inlet may be sealed around the catheter on all sides. Consequently, there may be no gap between the inlet and the catheter. As such, fluid may only pass from the sleeve/catheter into the inlet if it is within the catheter. The catheter may comprise a sleeve intake opening. The sleeve intake opening may be at or towards the distal end of the catheter. A side, e.g. a lateral side of the catheter may comprise the sleeve intake opening. The sleeve intake opening may be any suitable shape such as oval or circular. The sleeve intake opening may be adjacent to the inlet. The sleeve intake opening may permit the passage of fluid from the sleeve into the catheter. Thus, fluid may enter the catheter from the sleeve to join fluid travelling within the catheter. From here, the fluid may pass out the distal end of the catheter and into the inlet. Many other configurations may be possible to achieve the same effect, for example, the sleeve may be attached to the outer sides of the catheter itself.

Accordingly in one embodiment, there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag, wherein the sleeve comprises a distal end corresponding to the distal end of the catheter and the fluid collection bag comprises an inlet configured to allow fluid to enter the fluid collection bag from the distal end of the sleeve and optionally via the catheter, wherein the catheter assembly is configured to allow fluid to pass from the distal end of the catheter into the sleeve, or from the distal end of the sleeve into the catheter via a sleeve intake opening adjacent to the inlet.

The inlet of the fluid collection bag may be positioned at a corner of the peripheral bond. The fluid collection bag may comprise a neck adjacent to the inlet. The neck may comprise one or more projections extending into the fluid collection bag. The one or more projections may comprise regions of the peripheral bond that extend into the bag. The one or more projections may restrict fluid flow through a region of the bag adjacent to the inlet. The projections may be configured to provide gripping points adjacent to the inlet. Thus, the fluid collection bag resists the flow of fluid back towards the inlet and also is more easily handled by the user.

Any one or more of the one or more projections may have any one or more of the features of a projection as described below.

A projection may have any suitable size or shape. A projection may be triangular, square, rectangular, circular, ellipsoidal or irregularly shaped, preferably, a projection is triangular. A point on the projection furthest from an edge of the bag may be a (right-angled) corner in the projection. The projection may be a right-angled triangle. The hypotenuse of the projection may be aligned parallel to an edge of the bag. Thus, the projections can be suitably shaped to minimise loss of volume in the bag due to the projections. In addition, its shape can help to direct fluid flow within the bag, for example assisting flow into the bag and reducing flow out of the bag.

A projection may extend along no more than 90%, 80%, 70%, 60%, 50%, 40%, 30%, 20% or 10% of the length of an edge of the bag. A projection may extend by at least 10%, 20%, 30%, 40%, 50%, 60%, 70%, 80%, or 90% of the length of an edge of the bag. A projection may comprise a corner of the bag. A projection may not comprise from a corner of the bag. Thus, the projections can be flexibly sized and positioned to be most effective.

A projection may be a lateral projection extending from a left (or right) lateral edge of the bag. The lateral projection may be provided on the same side of the bag as the inlet. The lateral projection may extend along 10-40% of the length of the lateral edge of the bag, preferably 25%. The lateral projection may extend across 5-20% of the width of the bag, preferably 10%. The lateral projection may be a right-angled triangle. The right-angled vertex of the lateral projection may project into the bag. The right-angled vertex may be positioned below the inlet inside the bag. The right-angled vertex may be positioned 10% of the way from the upper edge to the base of the bag. The non-right-angled vertices of the lateral projection may be provided on the lateral edge of the bag from which the lateral projection extends. Thus, the lateral projection can assist in directing the flow of fluid into the bag and also helps prevent fluid flowing directly from the bag back up into the sleeve/catheter.

A projection may be an end projection extending from the upper edge or base of the bag. Preferably, the end projection extends from the upper edge. The end projection may be provided adjacent to the inlet. The end projection may extend along 40-95% of the width of the bag, preferably 90%. The end projection may extend from a corner of the bag. The end projection may extend down 5-20% of the length of the bag, preferably 10%. The end projection may be a right-angled triangle. The right-angled vertex of the end projection may project into the bag. The right-angled vertex may be positioned adjacent the inlet inside the bag. The right-angled vertex may be positioned 20% of the way across the width of the bag. The non-right-angled vertices of the lateral projection may be provided on the edge of the bag from which the end projection extends. Thus, the end projection can assist in directing the flow of fluid into the bag and also helps prevent fluid flowing directly from the bag back up into the sleeve/catheter.

In a preferred embodiment, the fluid collection bag comprises both a lateral projection and an end projection extending from the upper edge of the bag as described above. Thus, the two projections work together to assist the user in handling the bag and also to manage fluid flow into/out of the bag.

A projection may comprise a rib. The rib may resist bending or deformation in a given direction. The rib may facilitate folding of the fluid collection bag. The rib may assist the user in grasping or gripping the bag. The strengthening element may comprise a region of the projection that is thicker or thinner than the rest of the projection. The rib may be any suitable shape or size, such as a line which could be straight or curved. The rib may extend across the projection. The rib may extend from an edge of the bag. The rib may extend from a midpoint of the projection along the edge of the bag. The rib may extend to a point on the projection furthest from the edge of the bag, for example a right-angled vertex of a right-angled projection. The rib may be at an angle (i.e. not parallel or perpendicular to) an edge of the bag. Preferably, the lateral projection may comprise a rib. Thus, the mechanical properties of the projection can be controlled through use of a rib to the benefit of the user.

A projection may be a join that is independent from the peripheral bond of the fluid collection bag. A join may have any one or more of the features of a projection as described herein. A join can be beneficial to reduce the total area of the panels of the fluid collection bag that are sealed together.

The fluid collection bag may comprise a finger hole. The finger hole may be sized to allow a user to put their finger through the finger hole and handle the bag. Thus, the finger hole allows the bag to be handled more easily by a user. The finger hole may be provided at an edge of the bag. The finger hole may be provided in the peripheral bond of the bag. The finger hole may be provided at a corner of the bag. The finger hole may be provided at any of the following corners: where the left lateral edge meets the base; where the base meets the right lateral edge; where the right lateral edge meets the upper edge; or where the upper edge meets the left lateral edge. Where the finger hole is provided in the bag, the peripheral bond may be shaped to accommodate the finger hole. The peripheral bond may truncate a corner of the bag to accommodate the finger hole. The peripheral bond may project into the bag to accommodate the finger hole. As an alternative to the finger hole, the fluid collection bag may comprise a series of ridges or otherwise easily gripped surface or feature in the place of the finger hole to allow the bag to be easily handled.

The fluid collection bag may comprise more than one finger hole, for example at least two, three or more finger holes. One, two, three or more, or each finger hole may have any one or more of the features of the finger hole described above. Thus, easy handling of the bag is facilitated through positioning of finger holes or equivalent features which also does not restrict or affect the volume of liquid that can be contained by the bag.

The finger hole may be provided in a projection of the fluid collection bag. Preferably, the end projection as described above comprises a finger hole. The finger hole may be positioned adjacent to the right-angled vertex of the end projection.

The one or more finger holes may be configured to allow the bag to be rotated to a horizontal position wherein the base of the bag is aligned vertically. In a preferred embodiment, the fluid collection bag comprises a finger hole at either end of the right lateral edge, and/or a finger hole at either end of the left lateral edge, and/or a finger hole along the upper edge and a finger hole along the base. Advantageously, this allows the bag to be rotated in a controlled manner which aids with handling and using the bag.

The fluid collection bag may comprise a fill level indicator. The fill level indicator may comprise fill markers. The fill markers may allow the user to measure the volume of fluid contained within the fluid collection bag. The fill markers may be regularly spaced or irregularly spaced. The fill markers may correspond to an absolute volume of liquid contained within the bag. The fill markers may correspond to a proportion of the volume of the bag which is filled with fluid. The fill markers may indicate when a safe fill level of the bag has been reached. The fill level indicator may be positioned in a transparent or translucent region of the bag. Thus, the fill level indicator ensures the bag is not overfilled.

The upper edge of the peripheral bond may comprise a neck. The inlet may be located on the neck. The neck may project out in the plane of the fluid collection bag. The neck may project away from the fluid collection bag. The neck may span at least 10%, 20%, 30%, 40%, 50%, 60%, or 70% of the length of the upper edge. The neck may be positioned at any point on the upper edge, but preferably in the middle of the upper edge. The neck may be any suitable shape, but preferably it is triangular. The neck may comprise a left sloped edge and/or a right sloped edge. The left sloped edge and right sloped edge may project from the upper edge and converge at the inlet. Thus, the neck spaces the inlet from the rest of the bag which makes it easier for the user to locate. The provision of a neck can also help liquid entering the bag to be dispersed evenly within the bag.

The fluid collection bag may comprise an outlet to allow fluid to be drained from the bag. The inlet and the outlet may be positioned next to one another. The outlet may be sealed to allow fluid to fill the bag. The outlet may comprise a pull ring configured to be pulled and thereby form an opening in the bag. The pull ring may be configured to be moved in a direction away from the inlet to form the opening. The inlet may be configured to be folded along a neck fold-line. Once folded, the neck fold-line may form an edge of the bag. The pull ring may project beyond the edge of the bag defined by the neck fold-line.

Accordingly, a second aspect of the present invention provides a fluid collection bag for use with a catheter having a proximal end for insertion into the body and a distal end, the fluid collection bag comprising an inlet to allow fluid to enter the bag from the distal end of the catheter, and an outlet to allow fluid to be drained from the bag, wherein the inlet and the outlet are positioned next to one another, the outlet is sealed to allow fluid to fill the bag, the outlet comprises a pull ring configured to be moved in a direction away from the inlet to form an opening in the bag, and the inlet is configured to be folded so as to form an edge of the bag with the pull ring projecting beyond the edge.

Advantageously, the outlet is sealed so there is a low risk of leakage of fluid during filling the bag. In addition, the folding of the inlet reinforces the bag and inlet to ensure the opening is easily and reliably formed with clean edges that allow controlled emptying of the bag.

The inlet and outlet may be positioned on the same edge of the bag. The inlet may positioned centrally on an edge of the bag. The outlet may be position between the neck and a lateral edge of the bag. The outlet may be positioned at a corner of the bag. Thus, tearing/shear forces on the bag are minimised as they are concentrated on the corner of the bag this reduces the likelihood of the bag breaking and spilling liquid.

The outlet may be positioned distal from a finger hole of the bag, for example the finger hole described above in relation to the first aspect of the present invention. Where the outlet is positioned at or adjacent to an edge of the bag, that edge may be free from finger holes. Where the outlet is position at or adjacent an edge of the bag, a different edge of the bag (that is the right/left lateral edge) may comprise one or more finger holes. Thus, the finger holes are positioned on edges of the bag that generally do not correspond to the position of the outlet, this makes handling/emptying of the bag easier as the bag can be rotated and held using the finger holes to allow fluid to leave the bag due to gravity.

The outlet may project beyond an upper edge of the bag. The outlet may comprise a spout that projects from the upper edge of the bag. The pull-ring may be attached to the spout. The spout may be any suitable shape, for example rectangular, square, triangular or irregularly shaped, preferably it is rectangular or square. Advantageously, the outlet/spout projects from the edge of the bag and as such is easier to tear off the bag without damaging the rest of the bag and risking uncontrolled release of fluid from the bag.

The pull-ring may be disposed between the spout and inlet. The pull-ring may have any suitable shape, for example circular, elliptical, rectangular, square, triangular, or irregularly shaped. Thus, as the pull-ring is disposed between the spout and the inlet, when it is moved away from the inlet, shearing force from the movement is concentrated at the point the outlet meets the rest of the bag. This facilitates controlled tearing of the bag to create the opening.

The inlet may extend away from the bag, for example where the bag comprises a neck as described in relation to the first aspect of the invention. The outlet and the inlet may extend/project the same distance beyond an edge of the bag. The outlet and inlet/neck may have corresponding shapes. The outlet and inlet/neck may be shaped to fit into, around or next to one another. The pull-ring may be shaped to fit into, around or next to the inlet/neck. As such, there may be little or no gaps between the outlet/pull-ring and inlet/neck. Where the neck is triangular as described above in relation to the first aspect of the invention, the pull-ring may also be triangular in shape. The pull-ring may have the shape of a right-angled triangle with two perpendicular sides and a hypotenuse. The hypotenuse may be parallel and/or adjacent to one of the left sloped edge or right sloped edge of the neck. One of the perpendicular sides may form a side of the spout. As the inlet and outlet project the same distance beyond the edge of the pouch and have corresponding shapes, they protect one another from damage due to being caught on other objects.

The outlet may comprise a point of weakness. The point of weakness may be disposed in the peripheral bond at a point the outlet meets the peripheral bond. The point of weakness may be disposed at a point the outlet meets a lateral edge of the peripheral bond. The point of weakness may be arranged at the start or end of the opening. The point of weakness may be a notch, or thinned section, in the peripheral bond. The outlet may comprise one or more points of weakness as described above. The outlet may comprise a tear line. The tear line may comprise a weakened region of the outlet. An end of the tear line may comprise a point of weakness. Where the outlet comprises two or more points of weakness, a point of weakness may be positioned at either end of the tear line. The tear line may be configured to preferentially tear. The tear line may be formed by any suitable means, such as laser scoring. Thus, the provision of one or more points of weakness further assists controlled tearing of the bag by providing points in the outlet which preferentially break to form parts of the opening such as the start and/or end.

The sleeve may comprise a reservoir adapter. An end of the sleeve may comprise a reservoir adapter. The reservoir adapter may be configured to engage the fluid reservoir. The reservoir adapter may be configured to receive wetting fluid from the fluid reservoir. The reservoir adapter may be configured to detach from the fluid reservoir. The reservoir adapter may be configured to permit the catheter to pass through the reservoir adapter.

According to a broad aspect of the present invention, there is provided catheter assembly comprising a fluid reservoir, a catheter which comprises a proximal end for insertion into the body and a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

The catheter assembly may be a packaged catheter assembly comprising a pouch containing the fluid reservoir and catheter.

The sleeve and catheter may not be separatable. The sleeve may have a diameter that is no more than four times, three times or two times the diameter of the catheter.

Accordingly in one embodiment there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag and the sleeve has a diameter that is no more than four times the diameter of the catheter

The pouch may provide a sterile environment for the catheter. Accordingly, in one embodiment there is provided a sterile sealed packaged catheter assembly comprising a pouch containing a catheter assembly according to any of the first to thirteenth aspects described herein. Thus, in one embodiment there is provided a sterile sealed packaged catheter assembly comprising a pouch containing a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag.

According to a third aspect of the present invention, there is provided a packaged catheter assembly comprising a fluid reservoir, a catheter, and a pouch containing the fluid reservoir and catheter, wherein the catheter comprises a proximal end for insertion into the body, a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

Thus, wetting fluid is efficiently and easily introduced to wet the catheter as the reservoir adapter retains the fluid reservoir at one end of the sleeve. This ensures that wetting fluid is not wasted and also that wetting fluid is less likely to leak out and wet the external surface of the sleeve or pouch which may cause discomfort for the user.

The sleeve may comprise a proximal end and a distal end corresponding to the proximal end and distal end of the catheter respectively. The proximal end of the sleeve may comprise the reservoir adapter. The reservoir adapter may be positioned at or adjacent to the proximal end of the catheter. Thus, the reservoir is adapted to deliver wetting fluid to the proximal end of the catheter first, this ensures that the part of the catheter first introduced into the body is most likely to be adequately wetted. This reduces the risk of injury or discomfort during use of the catheter.

The reservoir adapter may be configured to receive the fluid reservoir. The fluid reservoir may be configured to fit into the reservoir adapter. The reservoir adapter may be configured to enclose at least part of the fluid reservoir. Thus, the reservoir adapter may be configured to enclose part of the fluid reservoir and receive it, this can be advantageous to help direct wetting fluid from the reservoir into the sleeve when the reservoir is burst at a point enclosed by the reservoir adapter.

The reservoir adapter may tubular. The reservoir adapter may comprise a bore therethrough. The bore may be sized to allow the catheter to pass through the reservoir adapter. The bore may be sized to allow the catheter and wetting fluid to pass through the reservoir adapter. The reservoir adapter may be configured to inhibit passage of the catheter through the reservoir adapter/bore while the fluid reservoir is engaged by the reservoir adapter. The fluid reservoir may be configured to block the catheter from passing through the reservoir adapter/bore. The fluid reservoir may be configured to block the catheter from passing through the reservoir adapter/bore if the fluid reservoir is engaged by the reservoir adapter. Thus, the catheter may be withdrawn from the sleeve through the reservoir adapter and bore, but only once the fluid reservoir is removed from the reservoir adapter. This helps to protect the catheter from dirt and damage prior to use.

The reservoir adapter may be cylindrical, or alternatively may be cuboid or any other suitable shape. The reservoir adapter may have a length in a direction parallel to the axis of the catheter and a width in a plane perpendicular to its length. The reservoir adapter may have a diameter or width at least twice a diameter of the catheter. The reservoir adapter may have a length no longer than half the diameter or width of the reservoir adapter. Thus, the reservoir adapter provides an end of the sleeve that projects outwardly so as to function as a handling element and be easy to identify and manipulate. This enables the proximal end of the catheter to be easily positioned for use without actually touching the catheter directly.

The reservoir adapter may comprise, or be referred to as, a handling element. The handling element may be configured to allow the user to move at least part of the sleeve along the catheter, preferably the proximal end of the sleeve. The reservoir adapter/handling element may have a higher rigidity than the sleeve and/or catheter. The reservoir adapter/handling element may comprise high density polyethylene (HDPE). Thus, the reservoir adapter may act as a handling element for the catheter and provide the user with a convenient point to control the use of the catheter and the movement of the sleeve up and down the catheter.

The reservoir adapter may comprise an inserter tip. The inserter tip may disposed at the proximal end of the reservoir adapter distal from the sleeve. The inserter tip may be configured to guide the passage of the catheter into the body. Thus, the reservoir adapter can be used by the user to ensure comfortable, safe and efficient use of the catheter.

The fluid reservoir may be configured to receive the reservoir adapter, preferably when the reservoir adapter comprises an inserter tip. The reservoir adapter may be configured to fit into the fluid reservoir. The fluid reservoir may be configured to enclose at least part of the reservoir adapter and preferably the inserter tip. Thus, the fluid reservoir can then protect the reservoir adapter. Where the reservoir adapter comprises an inserter tip, this is particularly useful as it can keep the tip clean and sterile before use.

The reservoir adapter may comprise a tube extending therefrom. The tube may define an aperture (e.g. the bore mentioned above) which permits the catheter to exit the sleeve. The tube may have any suitable cross-section but preferably it is the same shape as the catheter, that is preferably circular. The tube may have a diameter or width greater than the diameter of the catheter. The tube may have a diameter or width less than a diameter or width of the reservoir adapter. The tube may have a length no longer than 4×, 3×, 2× or 1× its diameter or width, preferably 1× its diameter or width. Thus, the tube creates a space between the reservoir adapter and the aperture through which the catheter exits the sleeve. This reduces the likelihood that the users hand blocks a view of the catheter leaving the sleeve and shields the catheter from direct contact with the user's hand or fingers which are holding the reservoir adapter as the catheter leaves the sleeve.

The reservoir adapter may comprise a distal end corresponding to an end of the reservoir adapter attached to the sleeve. The distal end may be circular or cylindrical. The distal end may have an annular cross-section. The distal end may comprise an internal diameter sized to allow the catheter and wetting fluid to pass therethrough. The distal end may have an outer structure that is any shape and optionally does not correspond to the shape of the inner structure. Thus, the sleeve and catheter are attached to the reservoir adapter and the catheter may pass through it.

The reservoir adapter may be configured to engage the fluid reservoir by any suitable means, such as push-fit, bayonet fit, or screw-fit engagement. The reservoir adapter may have an internal size and shape that matches the fluid reservoir. Thus, the fluid reservoir may be easily push-fit into the reservoir adapter.

The aperture and/or tube may engage the fluid reservoir. The aperture and/or tube may releasably engage the fluid reservoir. The aperture and/or tube may be configured to engage the fluid reservoir through any suitable means such as a push-fit, bayonet, or screw fitting. The fluid reservoir may be moveable to a position in which it cannot be removed from the sleeve. The fluid reservoir may be moveable to a position in which it can be removed from the sleeve. When fastened/engaged to the sleeve/reservoir adapter, the fluid reservoir may be moveable axially with respect to the sleeve. The fluid reservoir may configured to be removed from the sleeve through an axial movement and then a rotational movement. In one embodiment, the fluid reservoir is moveable axially with respect to a (screw) fitting of the sleeve (between a position in which the fluid reservoir does not engage the fitting and a position in which the fluid reservoir does engage the fitting). The aperture and/or tube may be in fluid communication with the fluid reservoir. Thus, the fluid reservoir is conveniently retained on the end of the sleeve prior to use such that it may deliver wetting fluid directly into the sleeve and can also be removed to allow the catheter to exit the sleeve. Further, the fluid reservoir is less likely to be unintentionally removed from the sleeve as it requires two different movements to be removed. The fluid reservoir may prevent the catheter from being withdrawn from the sleeve. The fluid reservoir may prevent the catheter from being withdrawn from the sleeve until after the fluid reservoir has released wetting fluid into the sleeve. A water-tight, and preferably sterile, seal may be provided between the reservoir adapter/sleeve and fluid reservoir. Thus, the catheter is always wetted prior to removal of the fluid reservoir and fluid released from the fluid reservoir does not leak out between the fluid reservoir and sleeve, this ensures it is most effectively used to wet the catheter. In addition, the sterility of the catheter is maintained prior to use which reduces the risk of infection to the user.

Consequently, in one embodiment there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag, wherein the sleeve comprises a handling element at one end corresponding to the proximal end of the catheter, the fluid reservoir is disposed on the handling element and the handling element releasably engages the fluid reservoir, wherein the fluid reservoir is movable axially with respect to the sleeve between a position where it can be removed from the sleeve and a position where it cannot be removed from the sleeve.

In another embodiment there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag, wherein the fluid reservoir is configured to prevent the catheter from being withdrawn from the sleeve.

The fluid reservoir may be a sachet. The fluid reservoir may be any suitable shape or size, such as square, rectangular, circular, ellipsoidal or irregularly shaped. Preferably, the fluid reservoir is elongate and optionally rectangular. The reservoir adapter may be configured to engage a first end of the fluid reservoir. The fluid reservoir may comprise a second end distal the first end. The fluid reservoir may have a length defined as the distance between the first end and the second end. The length of the fluid reservoir may be at least 50 mm, at least 60 mm, at least 70 mm, at least 80 mm, at least 90 mm, at least 100 mm, at least 120 mm or at least 140 mm. the length of the fluid reservoir may be no more than 160, 150, 140, 120, 100, 90 or 80 mm. The length of the fluid reservoir may be between 120 and 150 mm, e.g. 140 mm, which is typical for fluid reservoirs used for male intermittent catheters. Alternatively, the length of the fluid catheter may be between 70 and 90 mm, e.g. 80 mm. This length is typical for female intermittent catheters, but, because of the provision for the reservoir adaptor, which ensures that wetting fluid is not wasted, may be suitable even for male catheters (which are much longer than female catheters).

The fluid reservoir may be removable from the pouch. Where the fluid reservoir is removeable from the pouch, the fluid reservoir may comprise an activation marker. The fluid reservoir may be removable from the pouch with the reservoir adapter. The reservoir adapter may be configured to detach from the fluid reservoir only when outside the pouch. Thus, the user can more easily control when wetting fluid is released by first removing the fluid reservoir and reservoir adapter from the pouch and then releasing wetting fluid/detaching the fluid reservoir.

The fluid reservoir may be retained within the pouch. Where the fluid reservoir is retained in the pouch, the pouch may comprise a wetting marker. The fluid reservoir may be attached to the pouch. The fluid reservoir may be attached to the pouch by any suitable means, such as an adhesive, weld, or hook and hook/hook and loop type fasteners or the like. The fluid reservoir may be retained in the pouch by the jacket. The reservoir adapter may be configured to detach from the fluid reservoir if the reservoir adapter is removed from the pouch. Thus, the reservoir adapter may automatically detach which can be more convenient for the user. In addition, as the fluid reservoir is retained in the pouch, any leaks from it are contained within the pouch. In addition, there are less objects for the user to keep track of while using the catheter assembly.

The reservoir adapter may be configured to extend around the first end of the fluid reservoir. The reservoir adapter may be configured to extend along the fluid reservoir. The reservoir adapter may be configured to extend along at least 10%, 20%, 30%, 40% or 50% of the length of the fluid reservoir. The reservoir adapter may be configured to extend along no more than 60%, 50%, 40%, 30% or 20% of the length of the fluid reservoir. Preferably, the reservoir adapter is configured to extend along a third of the length of the fluid reservoir. Thus, the reservoir adapter ensures that the fluid reservoir is securely engaged and that wetting fluid is delivered efficiently from the fluid reservoir into the sleeve.

The reservoir adapter may comprise a proximal end distal from the sleeve. The proximal end may be configured to receive and/or retain the fluid reservoir. The proximal end may extend around and along the fluid reservoir as described above. Where the fluid reservoir is a rectangular sachet, the proximal end may comprise an elliptical annular cross-section. Thus, the fluid reservoir is engaged in a push-fit/interference fit with the reservoir adapter.

The reservoir adapter may comprise a midsection between the proximal end and distal end. The proximal end may be tapered, for example where the proximal end and distal end are different shapes/sizes. The proximal end may have a larger diameter than the distal end. The midsection may be configured to funnel wetting fluid towards the proximal end of the reservoir adapter. The midsection may comprise a convex surface. Thus, the midsection aids handling of the reservoir adapter by the user and also helps ensure wetting fluid passes through the reservoir adapter onto the catheter.

The fluid reservoir may be configured to release wetting fluid when activated by a user. The fluid reservoir may comprise an activation marker. The fluid reservoir may be configured to release wetting fluid upon activation of the activation marker. The activation marker may be on a surface of the fluid reservoir. The activation marker may comprise a region of the fluid reservoir that is identifiable to the user, for example through visual and/or tactile feedback. The activation marker may be located at a position on the fluid reservoir that is not enclosed by the reservoir adapter when the fluid reservoir is engaged by the reservoir adapter. Preferably, the activation marker may be at least visually recognisable, for example a different colour, or pattern, to the fluid reservoir. Thus, the activation marker is easily recognisable while the fluid reservoir is engaged by the reservoir adapter.

The fluid reservoir may be configured to break to release wetting fluid, for example by creating a tear in the fluid reservoir. The fluid reservoir may be configured to break at a point enclosed by the reservoir adapter when the fluid reservoir is engaged by the reservoir adapter. The reservoir adapter and/or fluid reservoir may comprise a sealing element. The sealing element may be configured to prevent wetting fluid from passing out of the proximal end of the reservoir adapter. The sealing element may be configured to provide resistance to movement of the reservoir adapter with respect to the fluid reservoir. The sealing element may comprise an O-ring provided on an internal surface of the reservoir adapter and/or an O-ring provided on an external surface of the fluid reservoir. Thus, wetting fluid is released into the reservoir adapter and does not leak out of the distal end of the reservoir adapter where it could cause discomfort/inconvenience for the user.

The fluid reservoir may be activated by a user by any suitable means, for example twisting the fluid reservoir, or applying a compressive force to the fluid reservoir. The fluid reservoir may be configured to break at a point adjacent to the aperture of the sleeve through which the catheter exits the sleeve. The fluid reservoir may be configured to release wetting fluid by pressing it against/towards the reservoir adapter. As the reservoir adapter/handling element is typically arranged at the proximal end of the catheter. The fluid reservoir may be configured to release wetting fluid by pressing it against/towards the proximal end of the catheter.

Accordingly in one embodiment, there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag, wherein the fluid reservoir is configured to release wetting fluid by pressing it against/towards the proximal end of the catheter.

In one embodiment, the fluid reservoir is configured to be pierced. The fluid reservoir may be pierced when it is pressed against/into the sleeve, for example against the reservoir adapter/handling element. The fluid reservoir may be pierced by a spike on the reservoir adapter. The fluid reservoir may then be detached from the reservoir adapter, for example by unscrewing the fluid reservoir from the reservoir adapter. Thus, the fluid reservoir may ensure timely and easy release of wetting fluid to wet the catheter.

Consequently, in one embodiment there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag wherein the sleeve comprises a handling element at one end corresponding to the proximal end of the catheter and the fluid reservoir is disposed on the handling element, wherein the fluid reservoir is configured to release wetting fluid by pressing the fluid reservoir against the handling element and the fluid reservoir is configured to be pierced by the handling element.

As mentioned above, the fluid reservoir may be movable between a position where it can be removed from the sleeve and a position where it cannot be removed from the sleeve. The fluid reservoir may be configured to release wetting fluid when it is in a position where it can be removed from the sleeve. The fluid reservoir may be configured to release wetting fluid before it is removed from the sleeve. The catheter assembly may be configured such that the fluid reservoir is inhibited from being removed from the sleeve until wetting fluid has been released from the fluid reservoir. Thus, the fluid reservoir may not be removed from the sleeve without it first being pierced/rupturing and releasing wetting fluid into the sleeve to wet the catheter for use.

Accordingly in one embodiment, there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag, wherein the catheter assembly is configured such that the fluid reservoir is inhibited from being removed from the sleeve until wetting fluid has been released from the fluid reservoir.

The fluid reservoir may be flexible. The fluid reservoir may comprise a flexible plastics material such as a hard moulded thermoplastic, for example HDPE.

The fluid reservoir may be secured to the pouch, e.g. secured by adhesive, by a weld, or by other means. As such, the fluid reservoir may be retained in the pouch after wetting, preventing misuse, e.g. by users taking the fluid reservoir out, pulling back the sleeve and seeking to wet the catheter manually.

The catheter assembly may comprise a jacket surrounding the fluid reservoir. The jacket may be configured to receive a wetting fluid from the reservoir. The jacket may be configured to direct wetting fluid into the sleeve to wet the catheter. Consequently, in a broad aspect of the invention there is provided a catheter assembly comprising a fluid reservoir and a catheter, the catheter having a proximal end for insertion into the body, a distal end, and a sleeve extending from the proximal end to the distal end; the assembly further comprising a jacket surrounding the fluid reservoir, the jacket being configured to receive a wetting fluid from the reservoir and direct the wetting fluid into the sleeve to wet the catheter.

The wetting fluid may be directed to the proximal end of the catheter before the distal end of the catheter. The wetting fluid may be directed into the proximal end of the sleeve. Thus, according to a fourth aspect of the invention, there is provided a catheter assembly comprising a fluid reservoir and a catheter, the catheter having a proximal end for insertion into the body, a distal end, and a sleeve extending from the proximal end to the distal end; the assembly further comprising a jacket surrounding the fluid reservoir, the jacket being configured to receive a wetting fluid from the fluid reservoir and direct the wetting fluid into the sleeve at the proximal end to wet the catheter.

Thus, the provision of a jacket around the fluid reservoir ensures that the wetting fluid released by the reservoir is directed efficiently onto the catheter to wet the catheter. This prevents loss/leaks of wetting fluid which can lead to insufficient wetting of the catheter rendering it unsuitable for use and/or causing discomfort/injury to the user during use.

The jacket also allows more design freedom in relation to the fluid reservoir and catheter assembly as a whole as the jacket controls the delivery of fluid to the sleeve/catheter. This allows the fluid reservoir/catheter assembly to be more user friendly.

The reservoir adapter may be configured to engage the jacket. The reservoir adapter may engage the fluid reservoir via the jacket. The reservoir adapter may be configured to receive wetting fluid from the fluid reservoir via the jacket. The jacket may be configured to block the catheter from passing through the reservoir adapter (until the jacket is detached from the reservoir adapter). The reservoir adapter may be configured to detach from the jacket (to allow the catheter to pass through the reservoir adapter). Thus, the jacket can provide a more flexible engagement between the reservoir adapter and fluid reservoir, for example, where the fluid reservoir is an inappropriate size to directly engage with the reservoir adapter.

The reservoir adapter may be configured to receive the jacket. The jacket may be configured to fit into the reservoir adapter. The reservoir adapter may be configured to enclose at least part of the jacket. Thus, the reservoir adapter may be configured to enclose part of the jacket and receive it, this can be advantageous to help direct wetting fluid from the jacket into the sleeve.

The jacket may be configured to receive the reservoir adapter, preferably when the reservoir adapter comprises an inserter tip. The reservoir adapter may be configured to fit into the jacket. The jacket may be configured to enclose at least part of the reservoir adapter and preferably the inserter tip. Thus, the reservoir adapter may be received into the jacket, this can be advantageous as the jacket can then protect the reservoir adapter. Where the reservoir adapter comprises an inserter tip, this is particularly useful as it can keep the tip clean and sterile before use.

The jacket may comprise a wetting aperture. The wetting aperture may be arranged at a position corresponding to a part of the fluid reservoir that is enclosed by the reservoir adapter. The wetting aperture may be arranged at an end of the fluid reservoir, preferably the first end referred to above. The wetting aperture may be configured to direct wetting fluid into the sleeve and/or onto the catheter. Thus, the wetting aperture helps ensure that wetting fluid released by the fluid reservoir is directed into the sleeve.

The fluid reservoir may be configured to rupture before the jacket. The jacket may be configured not to rupture under compression. The wetting aperture may be configured to allow fluids to exit and enter the jacket. Thus, the jacket remains intact when the user pinches the fluid reservoir to rupture it as any pressure build up is relieved through the wetting aperture.

The jacket may be impermeable to fluids, for example impermeable to liquid, in particular impermeable to water. The jacket may be formed of a material that is impermeable to fluids. The jacket may be formed from a flexible plastics material, for example a thermoplastic polyurethane (TPU) or low-density polyethylene (LDPE). Thus, the jacket can ensure that wetting fluid is directed into the sleeve and doesn't escape/leak out.

The jacket may be formed of a transparent or translucent material. The jacket may be formed of an opaque material. The jacket may comprise a window of transparent or translucent material at a position corresponding to the activation marker of the fluid reservoir. The jacket may comprise an activation marker. The activation marker may be on a surface of the jacket. The activation marker may comprise a region of the jacket that is identifiable to the user, for example through visual and/or tactile feedback. The activation marker may be located at a position on the jacket corresponding to a position of the fluid reservoir which when pressed will cause the reservoir to rupture. The activation marker may be in a position corresponding to the activation marker positioned on the fluid reservoir. Thus, the user can see the activation marker of the fluid reservoir through the jacket (through a window in the jacket if necessary), or alternatively can activate the fluid reservoir through an activation marker disposed on the jacket.

The jacket may be formed from two strips of material joined about their edges. Each strip of material may have a shape corresponding to the shape of the fluid reservoir. The wetting aperture may comprise a section around the edge of the jacket where the two strips of material are not joined together. The wetting aperture may comprise a section of the jacket which has been cut or punched to form the wetting aperture. Thus, the jacket and wetting aperture can be easily formed around the fluid reservoir if required.

The jacket may be secured to the pouch, e.g. secured by adhesive, by a weld, or by other means. As such, the jacket and the fluid reservoir within may be retained in the pouch after wetting, preventing misuse, e.g. by users taking the fluid reservoir out, pulling back the sleeve and seeking to wet the catheter manually.

In broad terms the invention also concerns a packaged catheter assembly comprising a pouch and the catheter assembly, wherein the pouch is configured to contain the catheter assembly within it. The fluid collection bag may be arranged within the pouch. The pouch may comprise an interaction region to form an opening in the pouch and allow the catheter and fluid collection bag to be removed from the pouch. The fluid collection bag may be arranged adjacent to the opening.

Thus, according to a fifth aspect of the present invention there is provided a packaged catheter assembly comprising a catheter, a pouch, and a fluid collection bag configured to receive fluid from the catheter in use, wherein: the catheter and fluid collection bag are arranged within the pouch; the pouch comprises an interaction region to form an opening in the pouch and allow the fluid collection bag and catheter to be removed from the pouch; and the fluid collection bag is arranged adjacent to the opening.

With this arrangement, the fluid collection bag protects the catheter from coming into contact with dirt as the pouch is opened and also helps prevent the user from accidentally touching the catheter during opening of the pouch. This helps to reduce risk of infection during use of the pouch as the catheter is less likely to be dirty.

The fluid collection bag may be provided in a stowed configuration, preferably while inside the pouch. The fluid collection bag may be configured to remain in the stowed configuration. In the stowed configuration, the fluid collection bag may be folded or rolled to reduce its size in any one dimension. The stowed configuration may be a folded or rolled configuration. In the stowed configuration, the fluid collection bag may be cylindrical in shape. Thus, the size of the fluid collection bag is minimised inside the pouch and this assists the user in removing the bag and other elements of the packaged catheter assembly from the pouch. The stowed configuration of the fluid collection bag further inhibits fluid flow onto the exterior surface of the fluid collection bag as it must penetrate between folds.

The pouch may comprise a flexible plastics material. For example, polypropylene (PP), polyethylene terephthalate (PET), low density polyethylene (LDPE), metalized polyester (MET PET) or orientated polypropylene (OPP).

The pouch may comprise two walls (a front wall and a rear wall) and a peripheral seal between the two walls. The peripheral seal may provide a sterile seal. Thus, the pouch may maintain the sterility of its contents.

The peripheral seal may form one or more edges of the pouch. The peripheral seal may comprise a left lateral edge, right lateral edge, an upper edge and a base of the pouch. The left and right lateral edges may be defined as the left and right edges when viewing the pouch with the front wall in front of the rear wall, the base at the bottom and the upper edge at the top. The pouch may be any suitable shape such as circular, elliptical, triangular, tubular, prismatic or polygon, preferably the pouch is rectangular. The pouch may have a width defined as the distance between the left lateral edge and right lateral edge of the pouch, the width preferably being at least 60, 70, 80, 90 or 100 mm. The pouch may have a width of no more than 110, 100, 90, 80, or 70 mm. Preferably, the width is between 90 and 110 mm, and most preferably about 100 mm. The pouch may have a height defined as the distance between the upper edge and base, the height preferably being at least 100, 125, 150, 175, 200 or 225 mm. The pouch may have a height of no more than 250, 225, 200, 175, 150, or 125 mm. Preferably, the height is between 120 and 140 mm, and most preferably it is about 130 mm.

The peripheral seal may comprise any one or more of: a weld; mechanical seal; heat seal; pressure seal; adhesive; solvent bond; ultraviolet bond; ultrasonic weld; laser weld; impulse weld; or friction weld. The peripheral seal may be re-sealable, for example through a resealable adhesive, an additional adhesive that is activated prior or during resealing, or a hook and loop/hook and hook fastener. Thus, the peripheral seal can be easily constructed to suit the specific needs of the pouch.

The pouch may comprise an interaction region defining a first point of contact for a user of the packaged catheter assembly. The interaction region may facilitate user access to the contents of the pouch. Actuation of the interaction region may create an opening in or near an upper edge of the peripheral seal. The catheter may be withdrawn through the opening. The catheter may be withdrawn through the opening. Thus, access to the pouch is provided at or near an upper edge of the pouch and as such, the fluid in the pool adjacent the base of the pouch is retained in the pouch while the catheter is withdrawn reducing the risk of spilling fluid outside the pouch.

The interaction region may comprise a breakable region of the peripheral seal. The breakable region may comprise a tear-away region which may comprise a tear line defining a line along which the pouch may be torn to at least partially separate the tear-away region from the remainder of the pouch, thereby creating an opening in the peripheral seal. The fluid collection bag may be arranged adjacent to the tear line. The tear-away region may comprise a tear start. The tear start may be provided at a first end of the tear-away region. The tear start may comprise a notch, or area of weakness, in one or both of the walls of the pouch. The tear start may define the first part of the pouch to be torn. The tear-away region may comprise a tear stop. The tear stop may be provided at a second end of the tear-away region. The tear stop may act to prevent or reduce the likelihood of the tear-away region being completely separated from the remainder of the packaging. A tear line may be defined between the tear start and the tear stop. The tear line may be configured to preferentially tear. The tear line may comprise a weakened region of the pouch. The tear line may be formed by any suitable means, such as laser scoring. Thus, the breakable region provides a convenient and easy means to open the pouch with minimal effort and no additional equipment/tools.

The tear-away region may be substantially triangular in shape having a sloped upper edge. The tear-away region may be tapered. The tear-away region may extend upwards more on one side than the other, preferably the tear-away region extends upwards more on the side corresponding to the tear start. The tear-away region may extend upwards no more than 200%, 100%, 75%, or 50% more on one side than the other. The tear-away region may extend upwards at least 25%, 50%, 75%, 100%, or 150% more on one side than the other. Preferably, the tear-away region extends upwards 50% more on one side than the other. Thus, the tear-away region is conveniently shaped to guide the user to grasp it on one side to open the pouch and also provides sufficient material for the user to securely grasp it on that side.

The interaction region may comprise an aperture. The aperture may be sized to allow a finger to pass through and grip the interaction region. Preferably, the aperture is located in the tear-away region. The aperture may be located on the same side of the pouch as the tear start. The aperture may be located adjacent to the tear start. The aperture may be any suitable shape, for example, circular, elliptical, square or irregularly shaped. Preferably, the aperture is circular. Thus, the user can more easily grasp the tear-away region and open the pouch.

The pouch may be formed from an opaque material. The pouch may have a textured or printed fabric-like appearance and/or feel. Where the pouch is opaque, the pouch may comprise a wetting marker to assist the user in locating the fluid reservoir within the pouch. Thus, the pouch is more comfortable and discreet for the user to carry in day-to-day life and the user can easily find and activate the fluid reservoir.

An axis parallel to the longest dimension of the stowed fluid collection bag may be arranged parallel to the tear line of the pouch and/or the opening formed in the pouch. This helps to protect the contents of the pouch from dirt when opening the pouch as the fluid collection bag provides an additional fluid impermeable barrier.

A first end of the stowed fluid collection bag (for example the inlet) may be arranged adjacent to one lateral edge of the pouch, or a first end of the opening. A second end of the stowed fluid collection bag (distal the first end) may be arranged adjacent a second end of the opening. For example, the first end of the stowed fluid collection bag may be arranged adjacent to the tear start. Thus, the fluid collection bag is positioned to ensure the distal end of the catheter is arranged along the same side of the pouch as the tear start. This helps the catheter to be withdrawn in a smooth and straight pulling action parallel to the lateral edge of the bag which reduces the force required to remove the catheter from the pouch.

The fluid collection bag may be configured to be withdrawn through the opening before the catheter. The fluid collection bag may be configured to withdraw the catheter from the pouch. Thus, the fluid collection bag facilitates removal of the catheter from the pouch without the user having to touch the catheter.

The fluid collection bag may be arranged to span across a majority of the width of the pouch. The fluid collection bag may span at least 50%, 60%, 70%, 80%, or 90% of the width of the pouch. Thus, the fluid collection bag forms a barrier across the opening in the pouch and protects the contents of the pouch.

The fluid collection bag may be arranged closer to the upper edge of the pouch than the base. The fluid collection bag may be arranged at least ½, ⅔, ¾ or ⅘ of the way from the base to the upper edge of the pouch. Thus, the fluid collection bag is arranged in the upper region of the pouch and close to the opening.

The fluid collection bag may obscure the catheter from the opening. The fluid collection bag may be arranged to span across a majority of the opening. The fluid collection bag may span at least 50%, 60%, 70%, 80%, or 90% of the opening. The fluid collection bag may obscure the rest of the contents of the bag from the opening. Thus, the user is less likely to see and then immediately grasp or touch the catheter itself.

The fluid collection bag may be configured to separate the two walls of the pouch. The fluid collection bag may be configured to separate the two walls of the pouch at the opening of the pouch. Thus, once the opening is formed in the pouch, the fluid collection bag removes the need for the user to separate the walls of the pouch which can be inconvenient or difficult, especially for users with reduced mobility.

The inlet of the fluid collection bag may be arranged adjacent to one lateral edge of the pouch and/or the opening. Where the packaged catheter assembly comprises a tear start, the inlet of the fluid collection bag may be arranged on the same side of the pouch as the tear start. Thus, the inlet is positioned to ensure the distal end of the catheter is arranged along the same side of the pouch as the tear start. This helps the catheter to be withdrawn in a smooth and straight pulling action parallel to the lateral edge of the bag which reduces the force required to remove the catheter from the pouch.

The fluid reservoir may be arranged within the pouch. The fluid reservoir may be arranged with its length parallel to one edge of the pouch. Preferably, the length of the fluid reservoir is parallel to the left and/or right lateral edge of the pouch. The fluid reservoir may be arranged adjacent to one edge of the pouch. Preferably, the fluid reservoir is arranged adjacent to the same edge of the pouch that the fluid reservoir's length is parallel to. Thus, the fluid reservoir is conveniently located against an edge of the pouch where it does not take up too much space.

The catheter may be arranged within the pouch in a shape having two opposing turns of at least 180 degrees along the catheter's length.

According to a broad aspect of the present invention, there is provided a packaged catheter assembly comprising a pouch and a catheter arranged within the pouch, wherein the catheter is arranged within the pouch in a shape having two opposing turns of at least 180 degrees along the catheter's length.

The pouch may comprise two walls and a peripheral seal between the two walls (as described above). Accordingly, in a sixth aspect of the present invention there is provided a packaged catheter assembly comprising a pouch having two walls and a peripheral seal between the two walls and a catheter arranged within the pouch, wherein the catheter is arranged within the pouch in a shape having two opposing turns of at least 180 degrees along the catheter's length.

Thus, the catheter is arranged in an “S-shape” which helps to retain the catheter (and anything connected to it) in a specific orientation while sealed within the pouch. This ensures the catheter is easy to remove from the pouch and use. In addition, it helps to protect the catheter from damage for example by reducing the likelihood of kinks forming along the catheter.

This is especially useful where the packaged catheter assembly is a packaged catheter assembly according to the third aspect of the invention as the catheter can easily be withdrawn to a straight (i.e. substantially straight) configuration by pulling the distal end of the catheter straight out of the opening of the bag, whilst leaving the proximal end of the catheter in its position. Then, the catheter assembly can be simply inverted and wetting fluid can flow straight down the sleeve. Obviously, this approach could be trickier if the catheter were curled up inside the pouch.

The two opposing turns may comprise a first turn and a second turn.

The distal end of the catheter may be adjacent to the inner (left or right) lateral edge near the upper edge of the pouch. The catheter may extend down the inside of the pouch. The two opposing turns may comprise a first turn arranged at an end of the pouch distal from an opening formed in the pouch. The first turn may be adjacent the base of the pouch. The catheter may then extend back up the pouch. The catheter may extend back up through a central region of the pouch, i.e. in a central half, third or quarter of the width of the pouch. The catheter may extend in a direction towards the distal end of the catheter. The two opposing turns may comprise a second turn arranged at an end of the pouch adjacent to an opening formed in the pouch. The second turn may be positioned adjacent the fluid collection bag. The second turn may be at a height in the pouch corresponding to the position of the distal end of the catheter. The catheter may then extend down the inside of the pouch. The catheter may extend adjacent to the inner edge opposite the edge adjacent to the distal end of the catheter (i.e. the right or left lateral edge). The proximal end of the catheter may be positioned approximately half the way down the height of the pouch. Thus, the catheter is safely stored within the pouch in a curved configuration which minimises risk of damage to the catheter—for example through kinking of the catheter—and also facilitates easy withdrawal of the catheter by pulling it by its distal end.

The catheter may be configured to be withdrawn from the pouch by the distal end of the catheter. The catheter may be configured such that during withdrawal of the catheter from the pouch the radius of curvature of a, or each, turn in the catheter is constant or increases. The two opposing turns may be configured to unravel simultaneously (during withdrawal of the catheter from the pouch). The two opposing turns may be configured to unravel after withdrawal of the catheter from the pouch. Thus, by pulling the catheter out of the pouch by its distal end, the catheter unravels without any one part of the catheter curving excessively, which could cause damage to the catheter.

The fluid reservoir may have a length of at least 20%, 30% or 40% the height of the pouch. The fluid reservoir may have a length no more than 50%, 40% or 30% the height of the pouch. Preferably the fluid reservoir has a length of 30% the height of the pouch. The fluid reservoir may have a width of at least 5%, 10%, 15% or 20% the width of the pouch. The fluid reservoir may have a width of no more than 25%, 20%, 15% or 10% the width of the pouch. Preferably, the fluid reservoir has a width of 15% the width of the pouch. The fluid reservoir (and reservoir adapter) may extend from the proximal end of the catheter to the base of the pouch. Thus, the fluid reservoir may be flexibly shaped and sized to conveniently fit within the pouch while allowing the catheter to remain in its S-shape.

A, or each, turn may comprise a turn of at least 180, 190, 200, 210 or 220 degrees. A, or each, turn may comprise a turn of no more than 230, 220, 210, 200 or 190 degrees. Preferably, a, or each, turn is between 180-220 degrees and most preferably a, or each, turn is 200 degrees. This ensures that the shape of the catheter makes best use of the pouch and that the catheter is protected.

A, or each, turn may comprise a constant curvature. Alternatively, a, or each, turn may comprise a variable curvature.

A, or each, turn may comprise a radius of curvature of at least ⅕, ¼ or ⅓ the width of the pouch. A, or each, turn may comprise a radius of curvature of no more than ½, ⅓, or ¼ the width of the pouch. Preferably, the radius of curvature of a, or each, turn is ⅓ the width of the pouch. A, or each, turn may span at least ¼, ⅓, ½, or ⅔ the width of the pouch. A, or each, turn may span no more than ¾, ⅔, ½, or ⅓ the width of the pouch. Preferably, a, or each, turn spans ⅔ the width of the pouch. Thus, the size of the turns and their respective radii of curvature are maximised to protect the catheter while providing space in the pouch for the other components of the catheter assembly as required.

The pouch may comprise an absorbent material. Accordingly, a seventh aspect of the present invention provides a packaged catheter assembly comprising a pouch and a catheter, wherein the pouch is configured to contain the catheter within it and wherein the pouch comprises an absorbent material.

Advantageously the provision of absorbent material allows any fluid in the pouch to be absorbed. This reduces leakage from the pouch making it more convenient and comfortable for a user to use and carry. A leak could occur prior to use (e.g. if someone inadvertently bursts a fluid reservoir); during use (e.g. when shaking the pouch to distribute wetting fluid within it or using the pouch as a surface on which to set up, or pack away the catheter assembly); or after use. In each of these situations, the provision of an absorbent material will be advantageous. Furthermore, as fluid in the pouch is absorbed by the absorbent material, then there is less fluid pooling within the bag and less fluid moving around within the pouch. This reduces noise associated with fluid sloshing within the bag and also makes the bag easier to carry as the weight distribution within it is more stable.

The absorbent material may be any suitable shape or size. The absorbent material may be any one of: an absorbent sachet; an absorbent layer; an absorbent strip. In one preferred embodiment the absorbent material is an absorbent layer. The absorbent layer may line at least part of an inside surface of the pouch. The absorbent layer may line at least 50%, 60%, 70%, 80%, or 90% of the inside surface of the pouch. The absorbent layer may line the whole inside surface of the pouch. The absorbent layer may line at least part of the front and/or rear walls of the pouch. The absorbent layer may line both the front and rear walls. The absorbent layer may comprise a single sheet of absorbent material folded to line both the front and rear walls of the pouch. Advantageously, the absorbent layer can be easily formed and added to the pouch construction as it can line the inside of the pouch and be folded inside.

The absorbent material may be configured to absorb at least 10%, 20%, 30%, 40%, 50%, 60%, 70%, 80%, or 90% of the liquid capacity of the fluid collection bag. In one embodiment, the absorbent material is configured to absorb 100% of the liquid capacity of the fluid collection bag. Thus, the absorbent material ensures that it can absorb (all) liquid within the fluid collection bag if required to reduce sloshing of liquid within the pouch/fluid collection bag.

The absorbent material may be configured to absorb at least 10%, 20%, 30%, 40%, 50%, 60%, 70%, 80%, or 90% of the liquid capacity of the fluid reservoir. In one embodiment, the absorbent material is configured to absorb 100% of the liquid capacity of the fluid reservoir. Thus, the absorbent material ensures that it can absorb liquid released from the fluid reservoir if required to dry components of the catheter assembly that must be handled by the user and to avoid liquid from leaking out of the pouch.

In another preferred embodiment the absorbent material may comprise an absorbent strip. The absorbent strip may be elongate. The absorbent strip may have a length and a width wherein the length is at least 2, 3, 4, 5, or 6 times larger than the width. The absorbent strip may be located adjacent to a mouth of the pouch. The absorbent strip may span the majority of the mouth of the pouch. The absorbent strip may span at least 50%, 60%, 70%, 80% or 90% of the width of the mouth. The absorbent strip may span no more than 90%, 80%, 70%, or 60% of the width of the pouch. The mouth may correspond to a part of the pouch which forms an opening to allow access to the contents of the pouch. The absorbent strip may line the mouth of the pouch. Two or more absorbent strips may be provided. For example, two opposing absorbent strips may be provided. One absorbent strip may be provided on each of two sides of the mouth of the pouch. Thus, absorbent strips may be used to help prevent fluid from leaking out of the pouch through the mouth of the pouch.

The absorbent material may comprise an absorbent polymer, a super absorbent, for example polyacrylic acid.

The absorbent strip may be positioned adjacent the tear line. The absorbent strip may be positioned below the tear line. The absorbent strip may span a majority of the tear line, for example at least 50%, 60%, 70%, 80% or 90% of the length of the tear line. Thus, the opening formed along the tear line is next to the absorbent strip and the absorbent strip may then absorb fluid within the pouch before it leaves through the opening.

One edge of the pouch may comprise a fold, i.e. it may be a folded edge. A folded edge may comprise a fold between the two walls. All edges other than the folded edge may be formed by the peripheral seal. The pouch may be formed by folding the two walls to form the folded edge. Thus, the pouch can be flexibly constructed from folding material if required.

The peripheral seal may be configured to be broken to allow the fold to be opened to a flat position.

In a broad aspect of the present invention there is provided a packaged catheter assembly comprising a catheter and a pouch; the pouch having two walls and a peripheral seal between the two walls, wherein one edge of the pouch comprises a permanent join between the two walls, and the remaining edges are sealed by the peripheral seal, which is configured to be broken to allow the pouch to be opened to a flat position, (by unfolding it about the permanent join). The permanent join is preferably a fold. Alternatively, it could be a bond formed, for example, by a weld or adhesive. A fold is preferred as by using a fold in a naturally flat sheet of material the pouch may be naturally biased towards the unfolded configuration.

Accordingly, in a eighth aspect of the present invention there is provided a packaged catheter assembly comprising a catheter and a pouch; the pouch having two walls and a peripheral seal between the two walls, wherein an edge of the pouch comprises a fold between the two walls, and the peripheral seal is configured to be broken to allow the fold to be opened to a flat position.

Advantageously, the inside of the pouch is clean and can be opened to present a flat surface which the user can take advantage of when using the catheter assembly, for example by resting items of the assembly on the surface during use.

The flat position may be defined as when the angle between the first wall and the second wall is less than 30, 20, 10, 5, or 1 degree. The flat position may be defined as when the angle between the first wall and second wall is zero. The angle between the first wall and second wall may be taken between lines extending from a point (that is on the pouch fold-line) at which the walls meet to respective points at the centre (of mass) of each wall. The angle between the first wall and second wall may be taken between lines extending from a point (that is on the pouch fold-line) at which the walls meet to respective points at the edge of each wall distal the point at which the walls meet. The flat position may be defined as when a vector normal to the plane of the first wall is substantially parallel to a vector normal to the plane of the second wall, that is within 45, 30, 20, 10 or 5 degrees of parallel to each other. The vector normal to the plane of the first/second wall may be a vector normal to a centre of the first/second wall. Thus, the two walls are aligned with each other in the flat position and therefore provide a flat surface for the user.

The fold between the two walls may define a pouch fold-line. The two walls may be formed from a single sheet of material. The pouch fold-line may correspond to a line of symmetry of the sheet of material. The pouch fold-line may correspond to the middle of the sheet of material. The pouch fold-line may be at a midpoint along a length of the sheet of material. The pouch fold-line may span a width of the sheet of material. Thus, the pouch is efficiently formed from a single fold of the sheet of material which maximises the volume contained within the pouch and while minimising the size of the pouch in any one dimension.

The fold may be configured to remain in the flat position. The fold may remain in the flat position if at least a threshold force is applied to separate the first and second walls. The threshold force may be less than 5 N, 1 N, 0.5 N, or 0.1 N. The fold may be configured to remain in a folded position. The fold may undergo plastic deformation when opened to the flat position and/or when closed to the folded position. Thus, the fold does not move from the flat position to the folded position and vice versa unless the user specifically moves it between the two positions making it more convenient to use.

The fold may be configured to open into the flat position from the folded position. Once the peripheral seal is broken, the fold may be configured to open into the flat position. The fold may be biased to the flat position. The fold may be elastically deformable. Thus, the fold may preferentially open flat once the peripheral seal is broken. This can make the pouch more easy to use for users with reduced mobility who may struggle to open the peripheral seal and also open the fold to the flat position.

The pouch may be configured to be closed by folding the pouch. The pouch may be configured to be closed by bringing the two walls together. The pouch may be configured to preferentially fold along the pouch fold-line. The pouch may be configured to be re-folded along the pouch fold-line. The peripheral seal may be re-sealable. Thus, the pouch may be folded back to its original closed configuration and the used parts of the catheter re-packaged within it to allow hygienic storage, transport and disposal of the catheter assembly.

Consequently, the pouch may have a closed configuration in which it is folded, and preferably sealed closed by the peripheral seal. The pouch may have an open configuration in which it is unfolded. The pouch may be initially provided in its closed configuration. The catheter assembly may be a sealed packaged catheter assembly.

The peripheral seal may provide a sterile seal. Thus, the pouch may maintain the sterility of its contents. In some embodiments, both the peripheral seal and peripheral bond of the fluid collection bag may provide a sterile seal. Thus, additional protection is provided to ensure the catheter remains sterile and furthermore, the pouch can be opened to provide a sterile surface which, if used and made dirty, does not affect the sterility of the catheter.

The peripheral seal may not provide a sterile seal. This can be advantageous where sterility of the catheter is maintained by other features of the catheter assembly and allows the pouch to be more cheaply constructed and further allows the peripheral seal to be more easily opened/an opening formed in the peripheral seal and re-sealed as described below.

The catheter may be arranged within the pouch/fluid collection bag such that it is curved at a point corresponding to a fold-line of the pouch and/or fluid collection bag. The catheter may be arranged with a tangent of the catheter at a fold-line of the pouch/fluid collection bag parallel to the fold-line. Thus, the catheter curves around the folds in the pouch and fluid collection bag to ensure that the catheter is at a reduced risk of kinking.

Each wall of the pouch may comprise a tab. Each tab may extend from an edge of the pouch. The tabs may be configured to be separated to create an opening in the peripheral seal of the pouch.

Accordingly, a ninth aspect of the present invention provides a packaged catheter assembly comprising a catheter and a pouch having two walls and a peripheral seal between the two walls, wherein the pouch is configured to contain the catheter within it, each wall comprises a tab extending from an edge of the pouch, and the tabs are configured to be separated in use to create an opening in the peripheral seal.

Advantageously, the tabs extend from the pouch and so are easy to grasp by the user. This makes opening the pouch easy even for users with reduced mobility.

The tabs may extend from the same edge of the pouch. The tabs may be overlapping. The tabs may extend from the same point on an edge of the pouch. The tabs may be completely overlapping. Thus, the tabs may be at the same position which reduces the overall size of the pouch, this makes it more discrete and convenient to carry before use.

The tabs extend from different points on an edge of the pouch. The tabs may extend from adjacent points on edge of the pouch. The tabs may extend from different edges of the pouch. Thus, the tabs are not positioned in the same place and each is therefore more easily identifiable and accessible to the user. This can be advantageous for users with reduced mobility or eyesight.

The tabs may extend from different, but overlapping points, on an edge of the pouch. Thus, the tabs can be easily separately identified, accessed and grasped on account of extending from different points, but the pouch can be more easily opened, on account of being pulled apart from an overlapping point on the edge of the pouch.

Each of the tabs may extend from an edge of the pouch that corresponds to a frangible portion of the peripheral seal. For example, the frangible portion as described above in relation to the eighth aspect. Thus, the tabs are suitably located to ensure the frangible portion breaks when the tabs are separated.

The pouch may be configured to be opened to a flat position, for example as described in the eighth aspect above. After opening of the pouch, the tabs may be disposed at opposite ends of the opened pouch.

Each tab may be configured to provide a handling point for the pouch. Thus, the tabs can be used to conveniently handle and move the pouch as may be required.

The tabs may be configured to allow the pouch to be closed. The tabs may be configured to be brought towards each other to close the pouch. This feature may preferably be used in combination with a peripheral seal that is re-sealable as described in relation to the eighth aspect. Thus, the tabs provide a handling point for the user to close the pouch, this can be particularly useful after using the catheter as the rest of the pouch may be dirty or soiled and so is undesirable to touch.

The tabs may be movable to a position where they do not project beyond an edge of the pouch. Each tab may be movable by deformation, folding or rolling of the tab. Each tab may be folded along a line corresponding to the edge of the pouch. Each tab may be folded against a wall of the pouch (e.g. the front wall or the rear wall). Both tabs may be moved in the same direction. Both tabs may be folded against the same wall of the pouch. Thus, the tabs may be moved to a position where they do not project beyond an edge of the pouch, this beneficially reduces the size of the pouch. In addition, it reduces the likelihood of the pouch being inadvertently opened, for example by the tabs becoming caught on another object.

Each of the tabs may be any suitable size or shape, such as rectangular, square, circular, elliptical, or irregular in shape. In one example, the tabs are trapezoid in shape with two sides, a base and a top, and a height. The length of the base may be greater than the length of the top. The length of the base may be at least twice as great as the height. A tab may be arranged with its respective base parallel to an edge of the pouch. Advantageously, the trapezoid shape of each tab makes it less likely to snag on other objects which could cause accidental opening of the pouch.

Each tab may comprise a gripping hole. Each gripping hole may provide a grippy surface or feature. Each gripping hole may comprise an aperture through the tab. Each gripping hole may be any suitable size or shape, for example, rectangular, square, circular, elliptical. In one embodiment, each gripping hole is in the shape of a stadium, that is a rectangle with semicircles replacing two opposite sides. The parallel sides of each gripping hole may be parallel to the base and top of each respective tab. The gripping hole of one tab may be in overlap with the other tab. Thus, the addition of a gripping hole to a tab allows the user to easily grasp it, of course a hole itself may not be necessary and instead any feature that allows the user to easily grasp the tab could be used.

According to a broad aspect of the present invention, there is provided a fluid collection bag for a catheter assembly, wherein the fluid collection bag comprises a hand strap.

‘Hand strap’ as used herein means a strap sized to be easily gripped by hand.

The fluid collection bag may be provided in a packaged catheter assembly, the packaged catheter assembly may comprise a pouch, a catheter, and the fluid collection bag. The packaged catheter assembly may comprise a fluid reservoir. The fluid collection bag may be configured to receive fluid from the catheter. The catheter, fluid reservoir and fluid collection bag may be arranged within the pouch. The fluid collection bag may comprise a front panel and a rear panel. A bond between the two panels may define a (internal) cavity.

The fluid collection bag of the broad aspect of the invention may comprise any of the features, including any optional features, as described in relation to the first to ninth aspects, or any other aspect, of the invention below.

According to a tenth aspect of the present invention, there is provided an intermittent catheter assembly comprising: a catheter having a proximal end for insertion into the body and a distal end; and a fluid collection bag arranged to receive fluid from the distal end of the catheter, wherein the fluid collection bag comprises a hand strap.

Advantageously, if the user is to hold the fluid collection bag when in use, the hand strap provides for a convenient and easy way of holding the fluid collection bag, where the hand strap can be temporarily worn around the user's wrist or be easily held. As such, the provision of a hand strap overcomes the problems of prior art intermittent catheters and ensures that the catheter is easy and convenient to use, in addition to being discrete and hygienic.

Further, the hand strap allows the fluid collection bag to be easily manipulated by the user during use. This means that the bag can be easily handled and manoeuvred by the user as is necessary. It also means that the user is no longer relying on their surroundings to use the catheter. For example, catheters described in the prior art are difficult to hold during use and so may need to be placed on the floor or leaning against an object to support the fluid collection bag during use. As stated above, this is associated with its own disadvantages. The present invention provides an assembly which can be easily and reliably held by the user.

Moreover, the provision of a hand strap means that the fluid collection bag can be hung from any suitable article, such as a wheelchair handle or bed post, when the catheter assembly is in use, i.e., during urination. Beneficially for the user, this means that the user does not need to transfer to a toilet cubicle or place the bag on the floor, which is plainly unhygienic, in order to urinate.

The fluid collection bag may receive fluid via an inlet arranged at an edge of the fluid collection bag. The hand strap may be arranged on the same edge as the inlet.

Advantageously, this means that when the fluid collection bag is held via the hand strap, for example by a user or by an article such as a wheelchair handle, the fluid entering the fluid collection bag flows away from the inlet, preventing back flow of the fluid toward the inlet and then the catheter, which can prevent the bag from being filled. Further advantageously, this provides for easier manipulation of the bag relative to the inlet. In use, it may be that the user will have to adjust the catheter and this will likely affect the position of the catheter relative to the fluid collection bag. As such, it is beneficial that the present invention provides for a fluid collection bag which can be easily manoeuvred and adjusted in use.

The fluid collection bag may comprise a front panel and a rear panel, and a bond between the two panels defining an internal cavity. The bond may be a peripheral bond. The peripheral bond may define a base, two lateral edges and an upper edge of the fluid collection bag. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag. The bag may have a width between the left lateral edge and right lateral edge of at least 8, 10, 12, 15 or 20 cm. The bag may have a width no more than 25, 20, 15, 10 or 8 cm. Preferably, the width is between 8 and 20 cm, more preferably between 10 and 15 cm, and most preferably about 12 cm. The base may define a bottom of the bag. The upper edge may define a top of the bag. The height of the bag from the base to the upper edge may be at least 20, 25, 30, 35 or 40 cm. The height of the bag may be no more than 45, 40, 35, 30, 25 or 20 cm. The height may be between 20 and 40 cm, 25 and 35 cm, or most preferably between 28 and 32 cm, for example 30 cm. The bag may be configured to hold at least 500, 700, or 1000 ml of fluid, and may hold no more than 1000 ml, for example, it may hold at least 700 ml of fluid.

The inlet may be arranged at the upper edge, left lateral edge, right lateral edge, or base of the fluid collection bag. Preferably, the inlet is arranged at the upper edge of the fluid collection bag. The hand strap may be arranged at the upper edge, left lateral edge, right lateral edge, or base of the fluid collection bag. Preferably, the hand strap is arranged at the upper edge of the fluid collection bag.

The hand strap may be arranged on a corner of the fluid collection bag where the upper edge and left lateral edge meet, a corner where the upper edge and right lateral edge meet, a corner where the left lateral edge and base meet, or a corner where the right lateral edge and base meet.

The fluid collection bag may comprise an outlet provided to allow fluid to pass out of the fluid collection bag after use. The hand strap may be arranged at an opposite end of the fluid collection bag to the outlet. Advantageously, this means that, in use, the bag is arranged such that the fluid entering the bag flows toward the outlet, preferably at or toward the base of the bag, irrespective of the position of the inlet. This facilitates draining of the bag during and/or after use. Moreover, when the user wishes to drain the bag, the bag can be held via the hand strap and the fluid discharged from the bag via the outlet, therefore, the user is not required to upend the bag to pour away the contents, which can be cumbersome.

The outlet may be arranged at the upper edge, left lateral edge, right lateral edge, or base of the fluid collection bag. The outlet may be arranged on the front panel or rear panel of the fluid collection bag, toward the upper edge, left lateral edge, right lateral edge or base. Preferably, the outlet is arranged on the front panel toward the base of the fluid collection bag.

The outlet may be arranged off-centre when the fluid collection bag is viewed with the rear panel behind the front panel. The outlet may be arranged off-centre, toward the left lateral edge or toward the right lateral edge.

The outlet may be arranged off-centre toward the side of the fluid collection bag on which the hand strap is arranged. For example, the hand strap may be arranged on the corner where the upper edge and right lateral edge meet, and the outlet may be arranged proximal to the base, off-centre toward the right lateral edge.

The hand strap may be joined to the fluid collection bag by any one or more of: a weld; mechanical seal; heat seal; pressure seal; adhesive; solvent bond; ultraviolet bond; ultrasonic weld; laser weld; impulse weld; or friction weld. In a preferred embodiment, the hand strap is joined to the bag by a weld. Advantageously, the hand strap may be joined to the bag easily and added to the fluid collection bag construction as part of conventional techniques for the manufacture of fluid collection bags.

The length of the hand strap may be between about 4 and about 30 cm, between about 5 and about 28 cm, between about 6 and about 26 cm, between about 7 and about 24 cm, between about 8 and about 22 cm, between about 9 and about 20 cm, between about 10 and about 19 cm, between about 11 and about 18 cm, between about 12 and about 17 cm, between about 13 and about 16 cm, between about 14 and about 16 cm, or about 15 cm. Advantageously, this provides a hand strap which can be arranged around many articles which a user may wish to hang the hand strap upon, such as a wheelchair handle or bed frame.

The length of the hand strap may be at least about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 22 cm, about 24 cm, about 26 cm, about 28 cm or at least about 30 cm.

The length of the hand strap may be no more than about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 22 cm, about 24 cm, about 26 cm, about 28 cm or no more than about 30 cm.

The hand strap may have a length equal to between about 20 and 100%, between about 30 and about 90%, between about 35 and about 80%, between about and about 70%, between about 40 and about 60%, between about 45 and about 55%, or about 50% of the length of the fluid collection bag.

The hand strap may have a length equal to at least about 20%, about 30%, about 40%, about 50%, about 60%, about 70%, about 80%, about 90%, or at least about 100% of the length of the fluid collection bag.

The hand strap may have a length equal to no more than about 20%, about 30%, about 40%, about 50%, about 60%, about 70%, about 80%, about 90%, or no more than about 100% of the length of the fluid collection bag.

The size of the hand strap may be fixed, i.e., the size of the hand strap may not be adjustable. This means that the length of the hand strap cannot be decreased or increased. This may provide a more secure hand strap because means for shortening or lengthening the strap can create areas of weakness in the hand strap, which can cause the strap to break after repeated use or increased tension.

Alternatively, the size of the hand strap may be adjustable such that the length of the hand strap can be decreased or increased.

The hand strap may have a width of between about 0.5 and about 4 cm, between about 0.6 and about 3.6 cm, between about 0.6 and about 3.2 cm, between about 0.7 and about 2.8 cm, between about 0.75 and about 2.4 cm, between about 0.8 and about 2 cm, between about 0.85 and about 1.6 cm, between about 0.9 and about 1.2 cm, between about 0.95 and about 1.1 cm, or about 1 cm.

The hand strap may have a width of at least about 0.5 cm, about 0.6 cm, about 0.7 cm, about 0.8 cm, about 0.9 cm, about 1 cm, about 1.5 cm, about 2 cm, about 2.5 cm, about 3 cm, about 3.5 cm, about 4 cm, about 4.5 cm, or at least about 5 cm. The hand strap may have a width of no more than about 0.5 cm, about 0.6 cm, about 0.7 cm, about 0.8 cm, about 0.9 cm, about 1 cm, about 1.5 cm, about 2 cm, about 2.5 cm, about 3 cm, about 3.5 cm, about 4 cm, about 4.5 cm, or no more than about 5 cm.

The width of the hand strap may be constant along its entire length.

The hand strap may be a loop of material or a strip of material. The hand strap may be a single, continuous loop of material.

Where the hand strap is a strip of material it may be attached at only one end to the fluid collection bag. Where the hand strap is a loop of material, it may be attached at two ends to the fluid collection bag. Where the hand strap is a loop of material, it may be joined to the fluid collection bag at two positions on the fluid collection bag. Each of the two positions may be substantially the same.

The hand strap may be joined to the fluid collection bag at a first position and at a second position. Preferably, the first position and the second position are substantially the same. The first position and the second position may each be on the upper edge, left lateral edge, base, right lateral edge, front panel, or rear panel of the fluid collection bag. The first position may be on a different edge or panel to the second position. For example, the first position may be on the front panel and the second position may be on the rear panel. As a further example, the first position may be on the upper edge and the second position may be on the rear panel.

The hand strap may be joined to the fluid collection bag via an aperture on the surface of the bag, and the hand strap is threaded through the aperture. The aperture may be provided on an edge of the fluid collection bag, preferably on the upper edge of the bag.

The hand strap may be formed of polyethylene terephthalate (PET), polypropylene or low-density polyethylene (LDPE).

The catheter may have a length of at least about 10 cm, about 15 cm, about 20 cm, about 25 cm, about 30 cm, about 35 cm, about 40 cm, about 45 cm, or about 50 cm.

The catheter may have a length of no more than about 70, 60, 50, 45, 40 or 35 cm.

The catheter may have a width of no more than about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 80 cm, about 90 cm, or no more than about 100 cm.

The intermittent catheter may be formed of a material of the group comprising: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, and water disintegrable or enzymatically hydrolysable material, or combinations, blends or co-polymers of any of the above materials.

The intermittent catheter may be formed of a material of the group comprising: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or co-polymers of any of the above materials.

The water disintegrable or enzymatically hydrolysable material may comprise a material of the group comprising: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

Preferably, the intermittent catheter is formed of a polyolefin material, in particular polyethylene and/or polypropylene.

Preferably, the intermittent catheter is formed of a thermoplastic elastomeric material.

At least a portion of the fluid collection bag may be formed from an opaque material or be coated to present the appearance of being opaque. At least 10%, 20%, 30%, 40%, 50%, 60%, 70% or at least 80% of the fluid collection bag may be formed from an opaque material or be coated to present the appearance of being opaque. No more than 10%, 20%, 30%, 40%, 50%, 60%, 70% no more than 80% of the fluid collection bag may be formed from an opaque material or be coated to present the appearance of being opaque. The fluid collection bag may be opaque at the inlet and/or outlet. Advantageously, this makes the contents of the pouch during and after use at least partially hidden and, therefore, more discrete for the user.

The inlet may be located at the end of a spout. The spout may extend from the upper edge, left lateral edge, base or right lateral edge of the fluid collection bag, but preferably from the upper edge. The diameter of the spout may remain constant for its entire length. The spout may comprise an end, distal to the fluid collection bag, which comprises the inlet. The spout may have a length, i.e., the distance from where the spout joins an edge of the fluid collection bag to the inlet, of between 2 and 14 cm, between 4 and 12 cm, between 6 and 10 cm, between 7 and 9 cm, or about 8 cm.

Advantageously, the spout makes it easier for the user to locate the inlet and provides a more secure connection between the catheter and the fluid collection bag.

The fluid collection bag may comprise an outlet valve configured to control release of fluid out of the fluid collection bag in use. The outlet valve may comprise: a valve base attached to a panel of the fluid collection bag, the valve base comprising a base opening for receiving a liquid output from the fluid collection bag; and an outlet element configured to be laterally rotatable relative to the valve base from a closed configuration to an open configuration.

The outlet element may comprise a handle operable to rotate the outlet element between the closed configuration and the open configuration. The handle may be planar.

The plane of handle may be aligned with, i.e., parallel with, the plane of the panel of the fluid collection bag to which the valve base is attached, when in the closed configuration.

According to a eleventh aspect of the invention, there is provided an intermittent catheter assembly comprising a catheter having a proximal end for insertion into the body and a distal end, a fluid collection bag arranged to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration.

Advantageously, the provision of the outlet element configured to be laterally rotatable relative to the valve base from a closed configuration to an open configuration means that the outlet valve is easily operable by a user. Of course, the downsides of a laterally rotatable outlet element associated with Foley catheters is irrelevant to intermittent catheters as the fluid collection bag is not attached to the user. The lateral rotation of the outlet element does not require movement of the fluid collection bag to be restricted, for example by holding the bag, to operate the valve. Thus, the outlet valve can be operated by a user with limited dexterity and by users to whom the fluid collection bag is not attached to their body, such as where the catheter is an intermittent catheter.

The outlet element may be rotatable such that the outlet element moves through an arc of rotation and wherein at a first end of the arc of rotation, the outlet element may be in the closed configuration, and at a second end of the arc of rotation, the outlet element may be in the open configuration.

The fluid collection bag may comprise a base and an upper edge and first and second lateral edges. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag.

The outlet valve may be arranged on the front panel, rear panel, upper edge, left lateral edge, right lateral edge, or base of the fluid collection bag. The outlet valve may be arranged on the front panel or rear panel of the fluid collection bag, toward the upper edge, left lateral edge, right lateral edge, or base. The means that the outlet valve may be arranged proximal to the upper edge, left lateral edge, right lateral edge, or base. Preferably, the outlet valve is arranged on the front panel toward the base of the fluid collection bag. It will be understood that the left and right edges need not be sharp edges and whilst a preferred embodiment comprises front and rear walls joined at their periphery to define the upper edge, left lateral edge, right lateral edge and base, alternatives could be envisaged, such as a cylindrical fluid collection bag, with circular upper and lower edges and left and right edges formed by the curved left and right sides of the cylinder.

In the closed configuration, the outlet element may substantially overlie a cavity of the fluid collection bag.

The outlet element may comprise a handle.

The handle may be rotatable from a position closer to the first lateral side to a position closer to the second lateral side.

The handle may be planar. Advantageously, this means that the handle is discreet and compact, and does not protrude away from the fluid collection bag. Thus, the handle is less likely to become caught on an object or be accidentally operated by the user, which may otherwise lead to unintentional opening of the outlet element when the fluid collection bag contains fluid.

The handle may comprise a finger-hole. The handle may be sized to allow at least one finger to pass through it. The handle may be sized to allow a user to grip it between at least their thumb and index finger. The handle may be any suitable shape, for example, circular, elliptical, square or irregularly shaped. Preferably, the aperture is elliptical. Thus, the user, in particular users with limited dexterity, can more easily grasp the outlet element. to operate the outlet valve.

The handle may be operable to rotate the outlet element between the closed configuration and the open configuration. Thus, the user, in particular users with limited dexterity, can more easily grasp the outlet element to operate the outlet valve.

The plane of the handle may be aligned with, i.e., parallel with, the plane of the panel of the fluid collection bag to which the valve base is attached, when in the closed configuration. The plane of the handle may be aligned with the plane of the panel of the fluid collection bag to which the valve base is attached, when in the open configuration. Advantageously, this means that the handle provides tactile feedback to the user to confirm when the handle, and therefore the outlet element, is in the open configuration or the closed configuration as the handle will be aligned with the valve base in each configuration. The user therefore has certainty that the outlet element is either open or closed, as desired.

Moreover, there is a reduced risk of the handle being caught on an object or be accidentally operated by the user, which may otherwise lead to unintentional opening of the outlet element when the fluid collection bag contains fluid.

The handle may rotate by at least about 90, about 120, about 150 or about 180 degrees between the closed configuration and the open configuration. Advantageously, this means that a rotation of significant magnitude is required to operate the outlet valve whilst till providing a rotation easily manageable by those with limited dexterity. This reduces the possibility of accidentally opening the outlet valve which may occur should the required rotation be smaller.

The handle may rotate by no more than about 90, about 120, about 150 or about 180 degrees between the closed configuration and the open configuration.

The handle may be inhibited from rotating more than 180 degrees clockwise or anti-clockwise due to obstruction by the valve base.

The handle may extend above the outlet element. The handle may extend above the valve base. When in the closed or open configuration, the handle may extend laterally further than the valve base. Advantageously, this means that the handle is sufficiently sized to be easily grasped by a user, in particular by a user with limited dexterity.

The valve base may be any suitable shape, for example, circular, elliptical, square, trapezoidal or irregularly shaped. Preferably, the valve base is trapezoidal.

The valve base may be attached to a panel of the fluid collection bag any one or more of: a weld; mechanical seal; heat seal; pressure seal; adhesive; solvent bond; ultraviolet bond; ultrasonic weld; laser weld; impulse weld; or friction weld. In a preferred embodiment, the valve base is attached to a panel of the fluid collection by a weld. Advantageously, the valve base may be joined to the bag easily and added to the fluid collection bag construction as part of conventional techniques for the manufacture of fluid collection bags.

The valve base may comprise a housing which houses a portion of the outlet element and in which the outlet element rotates. Thus, the housing may protect the outlet element from knocks against other objects, or similar. This prevents the outlet element becoming damaged and potentially inoperable or faulty.

The housing may define a cylindrical bore.

The handle may be connected to the outlet element.

The handle may be integrally formed with the outlet element. The handle and the outlet element may be formed as a single moulding.

The outlet element may have a generally tubular form.

The housing may have a generally tubular form.

The outlet element has a connector portion which may be may be configured for use with a connector having a connector diameter, the housing having a diameter marginally smaller than the connector diameter such that, on insertion of the connector onto a distal end of the housing in use, the connector portion can accommodate the connector.

The connector portion may comprise a flange distal to the outlet element. The flange may be arranged to inhibit a connector being extended toward the outlet element whilst still allowing the connector portion to accommodate the connector. The flange may be annular. The flange may be tapered inwardly as it extends away from the outlet element.

The valve base may be formed of a material which is more rigid than the relatively flexible material from which the fluid collection bag is formed. This assists the user in rotating the outlet element.

The valve base, housing and/or outlet element may be formed, for example, from polypropylene or High-density polyethylene (HDPE), acrylonitrile butadiene styrene (ABS) or polycarbonate. The materials of the valve base, housing and the outlet element may be chosen to have different shrinkage rates to allow for hot assembly of these two components to produce a good seal therefore preventing leakage. The use of different materials may also be different to avoid sticking between the two components over time.

The outlet element may be rotatable about a vertical axis of rotation when in use.

The outlet element may be rotatable about an axis parallel to the longest dimension of the fluid collection bag. The longest dimension of the fluid collection bag may be along the left lateral edge and along the right lateral edge, i.e., between the upper edge and the base.

The outlet element may be rotatable about an axis parallel to the plane of the panel of the fluid collection bag to which the valve base is attached, when in the closed configuration or the open configuration.

The valve base may be arranged on the front panel of the fluid collection bag. The outlet element may be rotatable about an axis parallel to the plane of a front panel of the fluid collection bag. The valve base may be arranged on the rear panel of the fluid collection bag. The outlet element may be rotatable about an axis parallel to the plane of a rear panel of the fluid collection bag.

The outlet valve may be arranged on the front panel or on the rear panel of the fluid collection bag.

The outlet element may protrude from an outer face of the valve base. The outlet element may protrude from an outer face of the valve base no more than 1 cm, 1.5 cm, 2 cm, 2.5 cm, 3 cm, 3.5 cm, 4 cm, 4.5 cm, or no more than 5 cm. The outlet element may protrude from an outer face of the valve base by at least 1 cm, 1.5 cm, 2 cm, 2.5 cm, 3 cm, 3.5 cm, 4 cm, 4.5 cm, or at least 5 cm.

When in the open configuration or in the closed configuration, the outlet valve may extend from the surface of the fluid collection bag no more than 1 cm, 1.5 cm, 2 cm, 2.5 cm, 3 cm, 3.5 cm, 4 cm, 4.5 cm, or no more than 5 cm.

When in the open configuration or in the closed configuration, the outlet valve may extend from the surface of the fluid collection bag by at least 0.5 cm, 1 cm, 1.5 cm, 2 cm, 2.5 cm, 3 cm, 3.5 cm, 4 cm, 4.5 cm, or at least 5 cm.

Advantageously, this provides a discreet and compact outlet valve which is not prone to accidental operating of the valve.

The catheter may have a length of at least about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 80 cm, about 90 cm, or at least about 100 cm.

The catheter may have a width of no more than about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 80 cm, about 90 cm, or no more than about 100 cm.

At least a portion of the fluid collection bag may be formed from an opaque material or be coated to present the appearance of being opaque. At least 10%, 20%, 30%, 40%, 50%, 60%, 70% or at least 80% of the fluid collection bag may be formed from an opaque material or be coated to present the appearance of being opaque. No more than 10%, 20%, 30%, 40%, 50%, 60%, 70% no more than 80% of the fluid collection bag may be formed from an opaque material or be coated to present the appearance of being opaque. The fluid collection bag may be opaque at the inlet and/or outlet. Advantageously, this makes the contents of the pouch during and after use at least partially hidden and, therefore, more discrete for the user.

The outlet valve may be formed of opaque materials.

The outlet valve may be arranged off-centre when the fluid collection bag is viewed with the rear panel behind the front panel. The outlet valve may be arranged off-centre, toward the left lateral edge or toward the right lateral edge.

The outlet valve may be arranged off-centre toward the side of the fluid collection bag on which the hand strap is arranged. For example, the hand strap may be arranged on the corner where the upper edge and right lateral edge meet, and the outlet valve may be arranged proximal to the base, off-centre toward the right lateral edge.

The fluid collection bag may be arranged to receive fluid from the distal end of the catheter via a valve assembly. The valve assembly may comprise a first configuration in which the valve assembly inhibits fluid flow in a direction from the fluid collection bag towards the catheter. The valve assembly may comprise a second configuration in which the valve assembly permits fluid flow in a direction from the fluid collection bag towards the catheter. Preferably, in the first configuration the valve assembly permits fluid flow in a direction from the catheter towards the fluid collection bag. In the second configuration, the valve assembly may inhibit fluid flow in a direction from the catheter towards the fluid collection bag.

Accordingly, a twelfth aspect of the present invention provides a catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag.

Advantageously, the two configurations of the valve assembly allow the user to efficiently fill the fluid collection bag and store fluid within it, as well as drain the bag in a controlled manner without the need to tear or open the bag. In addition, the valve assembly removes the need for a separate inlet and outlet in the fluid collection bag as a single opening in the fluid collection bag can operate as both the inlet and outlet, this reduces the manufacturing cost and complexity of the fluid collection bag. In addition, it makes the bag simpler to use and more robust.

The valve assembly may be provided in the first configuration. For example, the catheter assembly may be provided in the first configuration in a sealed pouch. The valve assembly may be configured to move from the first configuration to the second configuration. The valve assembly may be configured to move from the second configuration to the first configuration. The valve assembly may be configured to move reversibly between the first and second configurations. Thus, the valve assembly is reversible between the first and second configurations which allows the user to effectively re-seal the fluid collection bag if necessary. This can help prevent further leaks after draining the bag which can be common in bags where an outlet is opened irreversibly (for example by tearing the bag) and fluid lining the inside of the fluid collection bag slowly collects and flows out.

The valve assembly may be configured to resist movement from the second configuration to the first configuration. The valve assembly may be configured to resist movement from the first configuration to the second configuration. The valve assembly may remain in the first or second configuration. Thus, the valve assembly may not be reversibly switched between the first and second configurations as once in a configuration it resists movement back to the other configuration. This can help reduce the risk of inadvertently changing the configuration multiple times when only one change of configuration is required.

The valve assembly may comprise two passageways: a proximal passageway and a distal passageway. The proximal passageway may be configured to provide a fluid communication between the valve assembly and the catheter. The distal passageway may be configured to provide a fluid connection between the valve assembly and the fluid collection bag. The valve assembly may comprise a valve housing. The proximal and distal passageways may connect to opposite sides of the valve housing. The valve housing may be any suitable shape such as cuboid, spherical, cylindrical or irregularly shaped, preferably it is cylindrical. The proximal and distal passageways may connect to a side wall of the valve housing. The valve housing may have a diameter (or width) that is at least 1 cm, 1.5 cm, 2 cm or 3 cm. The valve housing may have a diameter that is no more than 4 cm, 3 cm, 2 cm or 1.5 cm. Preferably, the valve housing has a diameter of 3 cm. The valve housing may have an aspect ratio (length:width/diameter) of at least 1:1, 2:1 or 3:1. The valve housing may have an aspect ratio (length:width/diameter) of no more than 3:1, 2:1 or 1:1. Preferably, the valve housing has an aspect ratio of (length:width/diameter) 2:1. Thus, the valve assembly can be conveniently be placed in-line with the catheter and fluid collection bag via the passageways.

Each passageway may extend away from the valve housing by a distance that is no more than twice the diameter/width of the valve housing, and preferably a distance that is no more than the diameter/width of the valve housing. Thus, the valve assembly itself remains compact and the passageways are less likely to be damaged by bending forces experienced during use/manipulation of the catheter and fluid collection bag.

Each passageway may be tubular. Each passageway may have an internal diameter that is the same size or greater than an internal diameter of the catheter. Thus, the passageways have a lower fluid resistance than the catheter and do not unnecessarily inhibit the speed of fluid flow between the catheter and fluid collection bag.

The valve assembly may comprise a user operable control. The user operable control may be configured to move the valve assembly from the first configuration to the second configuration. The user operable control may be configured to move the valve assembly from the second configuration to the first configuration. The user operable control may be configured to reversibly move the valve assembly between the first and second configurations. Alternatively, the user operably control may not be reversible and may irreversibly switch the valve assembly from the first to the second configuration. The user operable control may comprise any suitable user actuable input such as a switch, button, lever, slider, knob. Preferably, the user operable control comprises a button. The button may be any suitable shape or size but is preferably the same shape as one side or face of the valve housing. The button preferably covers a majority of a side or face of the valve housing. Where the valve housing is cylindrical, the button is preferably cylindrical. One end of the valve housing may be an open end. The button may fill the open end of the valve housing. The button may be configured to move axially with respect to the (cylindrical) valve housing. The button may have an extended configuration. The extended configuration of the button may correspond to the first configuration of the valve assembly. In the extended configuration the button may extend beyond the open end of the valve housing. The button may have a depressed configuration. The depressed configuration of the button may correspond to the second configuration of the valve assembly. In the depressed configuration the button may be closer to the open end of the valve housing than when it is in the extended configuration. Preferably in the depressed configuration the button is flush with the open end of the valve housing. The valve assembly is therefore easy to use as the user can simply press down anywhere on one side of the valve assembly to operate the valve assembly, this is particularly useful where a user has reduced manual dexterity.

The user operable control may be biased to move the valve assembly to the first configuration. The user operable control may comprise a biasing element, for example a spring or other material/object that can be resiliently deformed by a user's hand or grip strength. The biasing element may be configured to bias the user operable control to move the valve assembly to the first configuration. The user operable control may be biased to keep the valve assembly in the first configuration. When the valve assembly is in the first configuration, the biasing element may be configured to bias the user operable control to keep the valve assembly to the first configuration.

The user operable control may be biased to move the valve assembly to the second configuration. The biasing element may be configured to bias the user operable control to move the valve assembly to the second configuration. The user operable control may be biased to keep the valve assembly in the second configuration. When the valve assembly is in the second configuration, the biasing element may be configured to bias the user operable control to keep the valve assembly to the second configuration. Thus, the user operable control helps to ensure that inadvertent switching between the first and second configurations is avoided.

The user operable control may comprise a switching point. The biasing element may be configured to reverse the bias of the user operable control when the user operable control is moved to and/or past the switching point. When the valve assembly is in the first configuration, the biasing element may be configured to bias the user operable control to move the valve assembly to the second configuration when the user operable control is moved to and/or past the switching point. When the valve assembly is in the second configuration, the biasing element may be configured to bias the user operable control to move the valve assembly to the first configuration when the user operable control is moved to and/or past the switching point. The switching point may correspond to the depressed configuration of the button. The switching point may correspond to a position in which the button is depressed further than the depressed configuration. Thus, the switching point ensures the valve assembly is only switched between the first and second configurations when the user intends which reduces the risk of leaks through the valve assembly.

The valve assembly may be configured to provide feedback as it switches between the first and second configurations, for example an audible/tactile output such as a “click” and/or a change of visual appearance. An audible and/or tactile output may be provided by any suitable means such as via a protrusion on the inside of the valve housing that overrides a detent on the button as the button moves with respect to the valve housing.

The valve assembly may be configured to indicate if it is in the first or second configuration. The user operable control may be configured to change the appearance of at least part of the valve assembly as the valve assembly is moved between the first and second configurations. The button may comprise a side wall that is a different colour or shade of colour from the valve housing. The side wall of the button may be visible when the button is in the extended configuration/the valve assembly is in the first configuration. The side wall of the button may be hidden by the valve housing when the button is in the depressed configuration/the valve assembly is in the second configuration. Thus, button provides a visual reference to the state of the valve assembly.

The valve housing may comprise a window. The window may be located in a side wall of the valve housing. The window may be configured to change appearance depending on if the valve assembly is in the first configuration or second configuration. The window may be located equidistance from each of the passageways. The window may be elongate. The window may comprise a length in a direction parallel to the axis of the (cylindrical) valve housing. The window may have a length that is approximately half the diameter/width of the valve housing. The window may have an aspect ratio (length:width) or 1:1, 2:1, 3:1 or 4:1, preferably 2:1. The window may be any suitable shape, such as circular, semi-circular, triangular, rectangular, or irregularly shaped, preferably, the window is stadium-shaped, that is rectangular semicircles replacing two opposite sides. The button may comprise a side wall with two regions which are visually distinct from one another, for example they may be a different colour, a different shade of colour and/or comprise different markings on them. One of the regions may correspond to the position of the window when the button is in the extended configuration and the other region may correspond to the position of the window when the button is in the depressed configuration. One region may be the same colour as the side wall of the button and the other may be a different colour/shade of colour as the side wall of the button. Alternatively, both regions may be a different colour or shade of colour from the side wall of the button. Thus, a window can provide a simple visual reference to the state of the valve assembly as a specific region of the button is visible through the window depending on the state of the button and therefore the valve assembly.

A top end of the button, that is the end that is outside the valve housing, may comprise an activation marker. The activation marker may comprise a visual indication to signal to the user how to use the button, for example it may comprise the word “PUSH” written on the button. The side wall of the button may also comprise an activation marker, for example an arrow pointing in the direction the button should be depressed. Thus, the operation of the button is intuitive for the use.

The valve assembly may comprise a valve body inside the valve housing. The valve body may be fluidly connected to the catheter (e.g. via the proximal passageway). The valve body may be fluidly connected to the fluid collection bag (e.g. via the distal passageway). The valve body may be connected inline between the catheter and fluid collection bag (e.g. via the proximal and distal passageways). The valve body may be configured to inhibit fluid flow in a direction from the fluid collection bag towards the catheter when the valve assembly is in the first configuration. The valve body may be configured to permit fluid flow in a direction from the catheter towards the fluid collection bag when the valve assembly is in the first configuration. The valve body may be configured to permit fluid flow in a direction from the fluid collection bag towards the catheter when the valve assembly is in the second configuration. The valve body may be configured to inhibit fluid flow in a direction from the catheter towards the fluid collection bag when the valve assembly is in the second configuration. The valve body may be coupled to the user operable control. Thus, the valve body enables the valve assembly to function as required.

The valve body may be configured to inhibit fluid flow in a direction from the distal passageway towards the proximal passageway when the valve assembly is in the first configuration. The valve body may be configured to permit fluid flow in a direction from the proximal passageway towards the distal passageway when the valve assembly is in the first configuration. The valve body may be configured to permit fluid flow in a direction from the distal passageway towards the proximal passageway when the valve assembly is in the second configuration. The valve body may be configured to inhibit fluid flow in a direction from the proximal passageway towards the distal passageway when the valve assembly is in the second configuration.

The valve body may be any suitable fluidic system to enable the valve assembly to operate as described above. The valve body may comprise a first check valve. The first check valve may be connected to the catheter (e.g. via the proximal passageway). The first check valve may be connected to the fluid collection bag (e.g. via the distal passageway). The first check valve may be configured to inhibit fluid flow in a direction from the fluid collection bag towards the catheter. The first check valve may be configured to permit fluid flow in a direction from the catheter towards the fluid collection bag. The valve body may be configured to connect the first check valve to the catheter and fluid collection bag when the valve assembly is in the first configuration. The valve body may be configured to disconnect the first check valve from the catheter and fluid collection bag when the valve assembly is in the second configuration. The valve body may be configured to connect the catheter and fluid collection bag by a tube (or a second check valve with the opposite polarity to the first check valve) when the valve assembly is in the second configuration. Thus, a simple check valve and tube may be used to operate the valve assembly. In an alternative, the first check valve could be operable to reverse its polarity in the second configuration. For example, a user operable control such as a lever or handle, could rotate the first check valve to reverse its polarity.

The valve body may be configured to move (e.g. axially) within the valve housing. The valve body may be configured to move the first check valve (e.g. axially) within the valve housing to connect and disconnect it from the proximal and distal passageways. The first check valve may be coupled to (and may move with) the button. The first check valve and tube (or second check valve) may be stacked (e.g. axially) within the valve housing. The valve body and user operable control/button may be integrally formed. As such, simple operation of the button allows the valve assembly to be operated with minimal additional components, of course, the first check valve, tube, and second check valve could be replaced with other fluidic components that operate in a similar manner to achieve the same result/benefits as the example given above.

The valve assembly may be directly connected to the fluid collection bag, for example the fluid collection bag may be directly connected to the distal passageway. Thus, the catheter assembly can form a compact unit and the operation of the valve assembly is more intuitive for the user as it is close to the bag.

The distal passageway may be connected to an access opening of the fluid collection bag. The access opening may be configured to allow fluid access to the inside of the fluid collection bag, thus the access opening may function as both an inlet and outlet of the bag. As such, any feature of an inlet or outlet described herein may equally apply to the access opening. The access opening may be formed of a flexible material. This is useful to allow the valve assembly to be moved with respect to the bag and furthermore to allow for more compact packaging of the fluid collection bag and valve assembly.

The valve assembly may be indirectly connected to the fluid collection bag, for example via an intervening tube or other fluidic component. This can allow the valve assembly to be more conveniently accessed by the user in some circumstances.

The catheter assembly may comprise a sleeve configured to enclose the catheter along the length of the catheter from the proximal end to the distal end. The sleeve may be attached to the valve assembly, preferably the sleeve is attached to the proximal passageway. The catheter and/or sleeve may be configured to deliver fluid to the valve assembly/proximal passageway. The catheter and/or sleeve may be configured to receive fluid from the valve assembly/proximal passageway. The sleeve may be configured to receive fluid from the valve assembly during draining of the fluid collection bag. The valve assembly may be directly connected to the sleeve, for example the sleeve may be directly connected to the proximal passageway. Thus, the sleeve can be used to ensure a simple and effective fluid connection between the catheter and the valve assembly.

The fluid collection bag may comprise a panel. The fluid collection bag may comprise an inlet (for example the access opening may function as an inlet). The fluid collection bag may comprise a base. The fluid collection bag may be configured to stand up on the base. Consequently, in a broad aspect, there is provided a catheter assembly comprising a fluid collection bag and a catheter; the fluid collection bag comprising a panel, an inlet to receive fluid from the catheter, and a base, wherein the fluid collection bag is configured to stand up on the base. The fluid collection bag may comprise two panels. The fluid collection bag may comprise a peripheral bond between the two panels. The two panels may be configured to form the base. The base may be distal from the inlet.

Accordingly, a thirteenth aspect of the present invention provides a catheter assembly comprising a fluid collection bag and a catheter; the fluid collection bag comprising at least two panels, a peripheral bond between the two panels, and an inlet to receive fluid from the catheter, wherein: the two panels are configured to form a base of the fluid collection bag distal from the inlet; and the fluid collection bag is configured to stand up on the base.

Advantageously, the fluid collection bag is free-standing and is more convenient to use as it does not need to be hung or held during filling. As it is free-standing, with the inlet distal the base, the bag also fills more efficiently with liquid and can ensure that more of the bag is filled with liquid. This means the fluid collection bag can have a smaller form factor which is cheaper, lighter and more discreet for the user.

The fluid collection bag may be any suitable shape or size, for example rectangular, circular, elliptical, cuboid, spherical, etc. The fluid collection bag may comprise a front panel and a rear panel. A peripheral bond may join at least part of the periphery of the front panel and rear panel to form the fluid collection bag. The peripheral bond may define a base, two lateral edges and/or an upper edge of the fluid collection bag. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag.

The fluid collection bag may have a width between the left lateral edge and right lateral edge. The width of the fluid collection bag may be at least 20, 25, 30 or 35 cm. The bag may have a width no more than 40, 35, 30, or 25 cm. Preferably, the width is between 30 and 35 cm, most preferably between 33 and 35 cm. The base may define a bottom of the bag. The upper edge may define a top of the bag. The length/height of the bag may be defined from the base to the upper edge. The height of the bag may be at least 15, 20, 25, 30, 35 or 40 cm. The height of the bag may be no more than 45, 40, 35, 30, 25, or 20 cm. The height may be between 20 and 40 cm, 25 and 40, 20 and 30 cm, 30 and 35 cm or most preferably between 33 and 35 cm, or 28 cm. Thus, preferably, the bag is square. The bag may be configured to hold at least 500, 700, or 1000 ml of fluid, and may hold no more than 1000 ml. For example, it may hold at least 700 ml of fluid.

The fluid collection bag may comprise a side panel, for example two side panels. The side panels may be configured to allow the front and rear panels to at least partially separate. Thus, the side panels allow the fluid collection bag to hold a greater volume of liquid.

Each side panel may be any suitable shape, for example lens shaped, rectangular or triangular. Preferably, the side panel is triangular, for example an isosceles triangle. Each side panel may have a height that is equal to the height of the fluid collection bag. The front panel may be joined to each side panel. The rear panel may be joined to each side panel. The front and rear panels may be joined to opposite sides of each side panel. Each side panel may have two equal length sides. The two equal length sides of a side panel may be bonded to the front and rear panels respectively. One side panel may be on a side of the fluid collection bag corresponding to the left lateral edge, and the other may be on the side corresponding to the right lateral edge. The side panels may be configured to form a base of the fluid collection bag. The side panels may be configured to allow the front and rear panels to separate further at the base of the fluid collection bag than at the upper edge of the fluid collection bag. The side panels may be configured to prevent separation of the fluid collection bag along the upper edge of the fluid collection bag. For example, the front panel and the rear panel may be directly joined to each other along the upper edge of the fluid collection bag. The two side panels may be configured to allow the front and rear panels to separate along the left lateral edge and/or right lateral edge of the fluid collection bag. The fluid collection bag may be wedge shaped. Thus, the side panels efficiently create additional volume within the bag and allow it to form a free-standing wedge shape.

The fluid collection bag may comprise a base panel. The base panel may span between the front panel and rear panel along the base of the peripheral bond. The base panel may be configured to form a base of the fluid collection bag. That is to say the base of the fluid collection bag may be formed by the front panel, the rear panel and the base panel, optionally also the two side panels. The base panel and side panels may be integrally formed. The base panel may be configured to allow the front panel and rear panel to separate to form the base of the fluid collection bag. The front panel may be joined to the base panel. The rear panel may be joined to the base panel. The front and rear panels may be joined to opposite sides of the base panel. The base may be formed by two flanges; the rear panel joined to the base panel (and/or side panels) may form one flange and the front panel joined to the base panel (and/or side panels) may form the other flange. Each flange may extend from one side of the upper edge of the peripheral bond to the other around the fluid collection bag, that is down a lateral edge (e.g. right lateral edge) along the base and back up the other lateral edge (e.g. left lateral edge). Consequently, the front panel and rear panel may be (directly) joined by the peripheral bond (only) along an upper edge of the fluid collection bag. Thus, the base panel and side panels may be used to form a base of the bag and allow it to be free-standing.

The base panel may be elongate. The base panel may comprise two elongate edges corresponding to the edges of the front and rear panels. The base panel may comprise two side edges corresponding to the edges of the two side panels. The base panel may be rectangular. The two flanges may extend from the two elongate edges. Thus, the base panel is efficiently attached to the fluid collection bag.

The base panel (and/or each side panel) may comprise a stowed configuration. In the stowed configuration, the base panel (and/or each side panel) may be configured to reduce the internal volume of the fluid collection bag. The base panel (and/or each side panel) may be folded in the stowed configuration. The base panel (and/or each side panel) may be folded between the front and rear panels. The base panel (and/or each side panel) may be configured to fold to allow the front and rear panels to come together. Thus, the base panel and/or each side panel may be folded to allow the fluid collection bag to reduce in size which assists with storage and transport of the bag making it lighter and more discreet for the user.

The base panel (and/or each side panel) may be configured to automatically unfold. The base panel (and/or each side panel) may be configured to unfold as liquid enters the fluid collection bag. Thus, the fluid collection bag can be automatically deployed into a standing configuration as liquid enters the bag, for example under the weight of liquid entering/inside the bag. This makes the bag easier to use as the user does not need to manually unfold it.

The fluid collection bag may comprise a flexible plastics material. For example, polypropylene (PP), polyethylene terephthalate (PET), low density polyethylene (LDPE), metalized polyester (MET PET) orientated polypropylene (OPP) or polyvinyl chloride (PVC). The fluid collection bag may be formed of an opaque material. At least part of the fluid collection bag may be transparent or translucent. The front panel of the fluid collection bag may be transparent or translucent. Thus, the fluid collection bag can conform to the shape of the catheter assembly, is convenient to carry and also provides a visual reference to the contents of the bag before, during and after use which makes it easier to use.

The fluid collection bag may comprise a fill level indicator. The fill level indicator may be configured to allow the user to visualise fluid within the bag. Where the fluid collection bag is formed of an opaque material, the fill level indicator may comprise a region of the fluid collection bag, such as part of the front panel, that is formed of a transparent or translucent material. The fill level indicator may be any suitable shape or size such as circular, semi-circular, triangular, rectangular or irregularly shaped, preferably it is rectangular. The fill level indicator may have a height that spans a majority of the height of the fluid collection bag, for example at least 50%, 60%, 70%, 80%, or 90% of the height of the fluid collection bag. Preferably, the fill level indicator has a height of over 90% of the height of the fluid collection bag. The fill level indicator may have a width that spans a minority of the width of the fluid collection bag, for example, no more than 50%, 40%, 30% or 20% of the width of the fluid collection bag. The fill level indicator may have a width of at least 10%, 20%, 30%, or 40% of the width of the fluid collection bag. Preferably, the fill level indicator has a width of 30-50%, or 35-40% of the width of the fluid collection bag, for example, 37%.

The fill level indicator may comprise fill markers. The fill markers may allow the user to measure the volume of fluid contained within the fluid collection bag. The fill markers may be regularly spaced or irregularly spaced. The fill markers may correspond to an absolute volume of liquid contained within the bag. The fill markers may correspond to a proportion of the volume of the bag which is filled with fluid. The fill markers may indicate when a safe fill level of the bag has been reached. The fill level indicator may be positioned in a transparent or translucent region of the bag. Thus, the fill level indicator ensures the bag is not overfilled.

The peripheral bond may comprise any one or more of: a weld; mechanical seal; heat seal; pressure seal; adhesive; solvent bond; ultraviolet bond; ultrasonic weld; laser weld; impulse weld; or friction weld. The peripheral bond is generally permanent but, in some embodiments, may be re-sealable, for example through a resealable adhesive, an additional adhesive that is activated prior or during resealing, or a hook and loop/hook and hook fastener. The peripheral bond may provide a water-tight, and preferably sterile, seal. Thus, the peripheral bong can be easily constructed to suit the specific needs of the catheter assembly and ensure the contents remains sterile if necessary.

The upper edge of the peripheral bond may comprise the access opening/inlet. The access opening may project out in the plane of the fluid collection bag. The access opening may project away from the fluid collection bag. The access opening may span no more than 30%, 20%, or 10% of the length of the upper edge. The access opening may be positioned at any point on the upper edge, but preferably on one side of the upper edge. The access opening may be any suitable shape, but preferably it is rectangular. Thus, the access opening is spaced from the rest of the bag which makes it easier for the user to locate and also allows it to flex and bend allowing movement of the valve assembly with respect to the fluid collection bag.

The fluid collection bag may comprise a finger hole. The finger hole may be sized to allow a user to put their finger through the finger hole and handle the bag. Thus, the finger hole allows the bag to be handled more easily by a user. The finger hole may be provided at an edge of the bag. The finger hole may be provided in the peripheral bond of the bag. The finger hole may be provided at a corner of the bag. The finger hole may be provided at any of the following corners: where the left lateral edge meets the base; where the base meets the right lateral edge; where the right lateral edge meets the upper edge; or where the upper edge meets the left lateral edge. Where the finger hole is provided in the bag, the peripheral bond may be shaped to accommodate the finger hole. The peripheral bond may truncate a corner of the bag to accommodate the finger hole. The peripheral bond may project into the bag to accommodate the finger hole. The finger hole may be any suitable shape but preferably has rounded smooth edges to allow it to be more comfortably grasped by the user. In one example, the finger hole is circular, in another it is stadium shaped, that is a rectangle with semicircles replacing two opposite sides. The finger hole may comprise two elongate straight edges with a length of one quarter to one third, for example 30%, of the height of the fluid collection bag. The elongate straight edges may be aligned parallel to the left and or right lateral edge of the fluid collection bag. The finger hole may have an aspect ratio (length:width) of at least 1:1, 2:1, 3:1, or 4:1, preferably the aspect ratio is 4:1. As an alternative to the finger hole, the fluid collection bag may comprise a series of ridges or otherwise easily gripped surface or feature in the place of the finger hole to allow the bag to be easily handled.

The finger hole may be configured to allow the bag to be rotated. Preferably, one finger hole is positioned at a corner of the bag that is opposite from the access opening/outlet. The finger hole may be adjacent to the base of the fluid collection bag. The finger hole may be closer to one lateral edge (e.g. the right lateral edge) than the access opening/outlet. The access opening/outlet may be closer to the other lateral edge (e.g. the left lateral edge) than the finger hole. Thus, the bag may be hung or held by the finger hole and gravity will drive fluid contained within the bag out through the access opening/outlet.

The fill level indicator may be positioned adjacent to the access opening/inlet/outlet, for example on the same side of the fluid collection bag as the access opening/inlet/outlet and spanning the majority of the height of the fluid collection bag. Thus, the fill level indicator ensures that during filling and draining the fluid within the fluid collection bag is visible as soon as it enters the bag and until it leaves the bag. This helps to ensure the fluid collection bag is completely emptied when draining the bag.

The access opening/outlet may be positioned distal from a finger hole of the bag. Where the access opening/outlet is positioned at or adjacent to an edge of the bag, that edge may be free from finger holes. Where the access opening/outlet is position at or adjacent an edge of the bag, a different edge of the bag (that is the right/left lateral edge) may comprise one or more finger holes. For example, the access opening/outlet may be provided at one corner of the bag and a finger hole may be provided at an opposite corner of the bag. Thus, the finger holes are positioned on edges of the bag that generally do not correspond to the position of the access opening/outlet, this makes handling/emptying of the bag easier as the bag can be rotated and held using the finger holes to allow fluid to leave the bag due to gravity.

The catheter and/or sleeve may be in fluid communication with the fluid collection bag via the valve assembly and preferably connected to the proximal passageway. The proximal passageway may be configured to allow fluid to enter the valve assembly from the distal end of the catheter and optionally via the sleeve. The proximal passageway may be connected to one end of the sleeve which corresponds to the distal end of the catheter. Thus, the proximal passageway allows liquid to pass into the fluid collection bag.

The catheter assembly may be configured to allow fluid to pass directly from the sleeve to the proximal passageway and/or directly from the catheter to the proximal passageway. One lateral side (e.g. a right lateral side) of the catheter may be attached to one side (e.g. a right side) of the proximal passageway. There may be a gap between a different lateral side (e.g. a left lateral side) of the catheter and a respective different side (e.g. a left side) of the proximal passageway. The distal end of the catheter may be within the proximal passageway. The sleeve may be sealed around a periphery of the proximal passageway. As such, fluid travelling within the sleeve is directed into the proximal passageway directly via the gap. In addition, fluid travelling within the catheter passes out the distal end and into the proximal passageway. In other examples, the catheter may be positioned differently and may be attached, for example, to the left side of the proximal passageway.

The catheter assembly may be configured to allow fluid to pass from the catheter into the sleeve. One lateral side (e.g. a right lateral side) of the catheter may be attached to a side (e.g. right side) of the sleeve. A distal end of the catheter may be within the sleeve. A distal end of the catheter may not be within the proximal passageway. The sleeve may be sealed around a periphery of the proximal passageway. As such, fluid travelling within the sleeve is directed into the proximal passageway directly and fluid travelling within the catheter passes out the distal end into the sleeve and then from there into the proximal passageway. In other examples, the catheter may be positioned differently and may be attached to a left side of the sleeve (or indeed a front side or rear side). Furthermore, the distal end of the catheter may be blocked and an outlet may be provided in a lateral side of the catheter to allow fluid to pass out into the sleeve.

The catheter assembly may be configured to allow fluid to pass from the sleeve into the catheter. One lateral side (e.g. a right lateral side) of the catheter may be attached to one side (e.g. a right side) of the proximal passageway. The proximal passageway may be sealed around the catheter on all sides. Consequently, there may be no gap between the proximal passageway and the catheter. As such, fluid may only pass from the sleeve/catheter into the proximal passageway if it is within the catheter. A side, e.g. a lateral side of the catheter may comprise a sleeve intake opening. The sleeve intake opening may be any suitable shape such as oval or circular. The sleeve intake opening may be adjacent to the proximal passageway. The sleeve intake opening may permit the passage of fluid from the sleeve into the catheter. Thus, fluid may enter the catheter from the sleeve to join fluid travelling within the catheter. From here, the fluid may pass out the distal end of the catheter and into the proximal passageway. Many other configurations may be possible to achieve the same effect, for example, the sleeve may be attached to the outer sides of the catheter itself.

The reservoir adapter may annular, for example it may be an O-ring. The reservoir adapter may comprise a bore therethrough. The bore may be sized to allow the catheter to pass through the reservoir adapter. The bore may be sized to allow the catheter and wetting fluid to pass through the reservoir adapter. The reservoir adapter may be configured to inhibit passage of the catheter through the reservoir adapter/bore while the fluid reservoir is engaged by the reservoir adapter. The fluid reservoir may be configured to block the catheter from passing through the reservoir adapter/bore. The fluid reservoir may be configured to block the catheter from passing through the reservoir adapter/bore if the fluid reservoir is engaged by the reservoir adapter. Thus, the catheter may be withdrawn from the sleeve through the reservoir adapter and bore, but only once the fluid reservoir is removed from the reservoir adapter. This helps to protect the catheter from dirt and damage prior to use.

The reservoir adapter may be configured to engage the fluid reservoir by any suitable means, such as push-fit, bayonet fit, or screw-fit engagement. The reservoir adapter may have an internal size and shape that matches the fluid reservoir. Thus, the fluid reservoir may be easily push-fit into the reservoir adapter.

The fluid reservoir may comprise a housing. The housing may be a jacket as described herein. The housing may be configured to surround the sachet, preferably the housing is tight fitting on all sides of the sachet. The housing may be configured to direct wetting fluid released from the sachet to wet the catheter. The housing may be fluid impermeable. The housing may be flexible. The housing may be formed from any suitable material such as silicone or a flexible plastics material like thermoplastic polyurethane (TPU) or low-density polyethylene (LDPE). The housing may be substantially the same shape as the sachet, for example rectangular. The housing may have rounded edges, this can help to ensure fluid does not get trapped in corners of the housing.

The reservoir adapter may engage the housing. The reservoir adapter may engage the housing via a wetting tube positioned at the proximal end of the sleeve. The housing may be integrally formed with the wetting tube. The housing may be a separate object from the wetting tube. The housing may be engaged by the wetting tube in a variety of ways such as push-fit, screw-fit or through an adhesive or weld, preferably the housing is push-fit into the wetting tube. The sleeve may extend around the wetting tube. The reservoir adapter may be fitted around the sleeve. The reservoir adapter may be fitted around the wetting tube. The reservoir adapter may be fitted around the housing. The reservoir adapter may urge any two or more of the sleeve, wetting tube and housing into engagement with one another. The Thus, the reservoir adapter can provide a simple but effective fluid tight seal between the fluid reservoir and sleeve.

The reservoir adapter may have an inner diameter that is less than an outer diameter of the wetting tube. The reservoir adapter may have an inner diameter that is less than an outer diameter of the housing. The reservoir adapter may be formed of a material that is more flexible than the wetting tube. The sleeve may be formed of a material that is more flexible than the reservoir adapter and/or wetting tube. Thus, the reservoir adapter stretches to accommodate the wetting tube and does not significantly reduce the size of the wetting tube when it is engaged by the reservoir adapter. This ensures fluid flow from the fluid reservoir to the sleeve is not unnecessarily inhibited.

The fluid reservoir may be configured to release wetting fluid when activated by a user. The fluid reservoir may comprise an activation marker. The fluid reservoir may be configured to release wetting fluid upon activation of the activation marker. The activation marker may be on a surface of the fluid reservoir, such as on the housing. The activation marker may comprise a region of the fluid reservoir that is identifiable to the user, for example through visual and/or tactile feedback. Preferably, the activation marker may be at least visually recognisable, for example a different colour, shade of colour, or pattern, to the rest of the fluid reservoir. Thus, the activation marker is easily recognisable while the fluid reservoir is engaged by the reservoir adapter.

The break-away region may form a part of the walls of the pouch, for example, the break-away region may form a part of the front wall and/or rear wall. Preferably, the break-away region is positioned adjacent to a lateral edge (e.g. right lateral edge) of the pouch. The break-away region may be any suitable shape or size, for example, rectangular, circular, ellipsoidal or irregularly shaped. Preferably, the break-away region is rectangular. The break away region may be defined by a tear line along which the pouch preferentially tears. The tear line may comprise a line of weakness in the walls of the pouch. The tear line may be formed by any suitable means, for example laser scoring. The tear line may span between two edges of the pouch, for example the upper edge and base. The tear line may be present on the front and/or rear wall of the pouch, preferably just the front wall. A tear start may be provided at a first end of the tear line. The tear start may comprise a notch, or area of weakness, in one or both of the walls of the pouch. The tear start may define the first part of the pouch to be torn. A tear stop may be provided at a second end of the tear line. The tear stop may act to prevent or reduce the likelihood of the tear-away region being completely separated from the remainder of the packaging. Thus, the break-away region can be used to form an opening in the pouch in a controlled manner. In addition, the tear line can only span one wall of the pouch which prevents the break-away region from becoming fully separated from the pouch which may be a nuisance for the user as they would then need to retain and dispose of an additional loose item.

The tear line may be flexibly positioned on the pouch. The tear line may span between two points on the edge of the pouch. The tear start may be positioned on an edge of the pouch. The tear stop may be positioned on an edge of the pouch. The tear start and tear stop may be positioned on opposite edges of the pouch, preferably the upper edge and base respectively. The tear line may be positioned off-centre with respect to the width of the pouch. Preferably, the tear line is positioned 70-90% across the width of the pouch from one lateral edge to the other, (e.g. left lateral edge to right lateral edge). The break-away region may therefore correspond to 30-10% of the width of the pouch. As such, a break-away region is formed which is a minority of the width of the pouch and this ensures that the contents of the pouch do not fall out in an uncontrolled manner as soon as the pouch is opened.

The break-away region may be the same colour, a different shade of colour or a different colour to the rest of the pouch. Thus, the break-away region can either be made more discreet or more obvious depending on the needs of the packaged catheter assembly.

The break-away region may comprise a pull-ring. The pull ring may be arranged adjacent to the tear start. The pull ring may be sized to allow a finger to pass through it. The pull ring may be configured to tear the pouch along the tear line. Thus, the pull ring makes it easier for the user to open the pouch by providing a ring which they can easily grip and pull, of course a ring itself may not be necessary and instead any feature the user can easily grasp could be used.

The pull ring may be a different colour, or shade of colour, from the walls of the pouch. Thus, it is more easily identifiable for the use.

The break-away region may comprise an opening marker configured to provide guidance to the user about how to open the pouch. The opening marker may direct the user to tear the pouch along the tear line, for example by grasping and pulling on the pull ring. The opening marker may comprise an arrow. The arrow may point from the tear start towards the tear stop along the tear line. The opening marker may provide visual guidance to the user, or may provide tactile guidance or otherwise for example using a region of the pouch that is a different texture to the rest of the pouch.

The fluid collection bag may be contained within the pouch. The fluid collection bag may be provided in a stowed configuration, preferably while inside the pouch. The fluid collection bag may be configured to remain in the stowed configuration. In the stowed configuration, the fluid collection bag may be folded or rolled to reduce its size in any one dimension. The stowed configuration may be a folded or rolled configuration. Preferably, the fluid reservoir is folded along its width and height when in the stowed configuration. Thus, the fluid collection bag is stored in a compact manner that allows the pouch to also be a small size and convenient for the user to carry on their person.

The access opening may be provided in a folded configuration within the pouch. The valve assembly may overlap the fluid collection bag within the pouch. The fluid reservoir may be contained within the pouch. The fluid reservoir may be positioned adjacent to the valve assembly in the pouch, for example beneath the valve assembly. The catheter may be provided in a curved and/or coiled configuration. The distal end of the catheter may be arranged adjacent to the valve assembly/proximal passageway. The proximal end of the catheter may be arranged adjacent to the fluid reservoir/reservoir adapter/wetting tube. The sleeve may comprise a turn between the wetting tube and the proximal end of the catheter. The turn may be at least 90 degrees, 120 degrees, 150 degrees or 180 degrees, preferably the turn is 180 degrees. Preferably, the turn is a hairpin turn. Thus, the packaged catheter assembly is arranged in a compact manner that is easy to access and use.

The fluid reservoir may be in a position within the pouch corresponding to the tear line. The fluid collection bag may be in a position within the pouch corresponding to the tear line. Thus, when the tear line is torn, the fluid reservoir and fluid collection bag are accessible for the user to grasp and remove from the pouch.

The catheter assemblies/packaged catheter assemblies of the first to thirteenth aspects may include any one or more features of a catheter assembly/packaged catheter assembly as defined in broad terms, or according to any other of the first to thirteenth aspects set out above. The catheter assemblies/packaged catheter assemblies of the first to thirteenth aspects may comprise any of the optional features of the others of the first to thirteenth aspects without necessarily including all the features required of them. That is to say, an optional feature which happens to be set out following one particular aspect does not necessarily apply only to that aspect, so, for example, the disclosure provides for the packaged catheter assembly of the sixth aspect comprising a pouch having two walls and a peripheral seal between the two walls and a catheter arranged within the pouch, wherein the catheter is arranged within the pouch in a shape having two opposing turns of at least 180 degrees along the catheter's length, further comprising a fluid collection bag comprising an inlet and one or more projections as described in relation to the first aspect.

According to a fourteenth aspect of the present invention there is provided a method of forming a catheter assembly comprising the steps of providing a catheter having a proximal end for insertion into the body and a distal end, arranging a fluid collection bag to receive fluid from the distal end of the catheter, enclosing the catheter along the length of the catheter from the proximal end to the distal end with a sleeve, and arranging a fluid reservoir at the proximal end of the catheter, wherein the fluid reservoir is configured to release wetting fluid into the sleeve to wet the catheter and wherein the sleeve is in fluid communication with the fluid collection bag.

The method of the fourteenth aspect of the invention may be a method of forming the catheter assembly of the first aspect of the invention, which, of course, may include any optional feature outlined above.

According to a fifteenth aspect of the present invention, there is provided a method of forming a fluid collection bag, the method comprising providing a bag, forming an inlet in the bag to allow fluid to enter the bag, and forming a sealed outlet in the bag next to the inlet, wherein the outlet comprises a pull ring configured to be moved in a direction away from the inlet to form an opening in the bag, and the inlet is configured to be folded so as to form an edge of the bag with the pull ring projecting beyond the edge.

The method of the fifteenth aspect of the invention may be a method of forming a fluid collection bag of the second aspect of the invention, which, of course, may include any optional feature outlined above.

The method may comprise forming a finger hole in the bag. The finger hole may be formed at a point distal from the outlet, for example on a different edge of the bag from the outlet. Thus, the finger hole can be more easily use to handle the bag and direct fluid flow out of the outlet.

The method may comprise providing front and rear panels of the fluid collection bag as described in relation to the first aspect of the invention above. The outlet and/or inlet may be provided integrally with the front and rear panels. The outlet and/or inlet may be formed by sealing the front and rear panels together and creating the peripheral bond. Thus, the inlet and/or outlet and fluid collection bag may be formed in a single step which simplifies manufacture.

The method may comprise forming a point of weakness in the outlet. The method may comprise forming a point of weakness at either side of the outlet. The method may comprise forming a tear line extending from at least one point of weakness in the outlet. The tear line may be formed by laser scoring as described in the second aspect of the invention above.

According to a sixteenth aspect of the present invention, there is provided a method of forming a packaged catheter assembly comprising the steps of providing a fluid reservoir, a catheter, and a pouch, and arranging the fluid reservoir and catheter within the pouch; wherein the catheter comprises a proximal end for insertion into the body, a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

The method of the sixteenth aspect of the invention may be a method of forming the packaged catheter assembly of the third aspect of the invention, which, of course, may include any optional feature outlined above.

The method may comprise arranging the reservoir adapter at the end of the sleeve. The method may comprise inserting the fluid reservoir into the reservoir adapter so as to block/inhibit the passage of the catheter therethrough. Thus, the catheter is protected by the fluid reservoir/reservoir adapter.

The method may comprise inserting the reservoir adapter into the fluid reservoir. The method may comprise inserting an inserter tip of the reservoir adapter into the fluid reservoir. Thus, the reservoir adapter is protected by the fluid reservoir.

The method may comprise attaching the fluid reservoir to the pouch. Thus, the fluid reservoir can be retained by the pouch.

The method may comprise forming a housing around a sachet. The method may comprise attaching the housing to the end of the sleeve using the reservoir adapter.

According to an seventeenth aspect of the present invention there is provided a method of forming a catheter assembly, the method comprising providing a fluid reservoir surrounded by a jacket and a catheter; wherein the catheter comprises a proximal end for insertion into the body, a distal end, and a sleeve extending from the proximal end to the distal end; wherein the jacket is configured to receive a wetting fluid from the reservoir and direct the wetting fluid into the sleeve at the proximal end to wet the catheter.

The method of the seventeenth aspect of the invention may be a method of forming the catheter assembly of the fourth aspect of the invention, which, of course, may include any optional feature outlined above.

The method may comprise forming the jacket around the fluid reservoir. The method may comprise arranging two strips of material, one on either side of the fluid reservoir. The method may comprise joining the strips of material around their edge to form the jacket with the fluid reservoir within it. Thus, the jacket is conveniently formed around the fluid reservoir.

The method may comprise forming a wetting aperture in the jacket. The method may comprise providing a reservoir adapter at an end of the sleeve and inserting the jacket (containing the fluid reservoir) into the reservoir adapter.

The method may comprise inserting the reservoir adapter into the jacket, for example into the wetting aperture. The method may comprise inserting an inserter tip of the reservoir adapter into the jacket, for example into the wetting aperture. Thus, the reservoir adapter is protected by the jacket.

The method may comprise attaching the jacket to the pouch. Thus, the fluid reservoir can be retained by the pouch via the jacket.

According to a eighteenth aspect of the present invention there is provided a method of manufacturing a packaged catheter assembly, the method comprising the steps of providing a catheter, a pouch, and a fluid collection bag configured to receive fluid from the catheter in use, arranging the catheter and fluid collection bag within the pouch wherein the pouch comprises an interaction region to form an opening in the pouch and allow the fluid collection bag and catheter to be removed from the pouch, and the fluid collection bag is arranged adjacent to the opening.

The method of the eighteenth aspect of the invention may be a method of manufacturing the packaged catheter assembly of the fifth aspect of the invention, which, of course, may include any optional feature outlined above.

The method may comprise arranging the catheter in the pouch and then arranging the fluid collection bag in the pouch. This can ensure the fluid collection bag remains adjacent to the opening.

The method may comprise attaching the catheter to an inlet of the fluid collection bag. The method may comprise arranging the inlet on one side of the fluid collection bag.

According to a nineteenth aspect of the present invention there is provided a method manufacturing a packaged catheter assembly, the method comprising providing a pouch having two walls and a peripheral seal between the two walls and a catheter, arranging the catheter within the pouch, and forming two opposing turns of at least 180 degrees along the catheter's length.

The method of the nineteenth aspect of the invention may be a method of manufacturing the packaged catheter assembly of the sixth aspect of the invention, which, of course, may include any optional feature outlined above.

The method may comprise forming a turn in the catheter during the step of arranging the catheter within the pouch. The method may comprise forming two opposing turns in the catheter before arranging the catheter in the pouch.

According to a twentieth aspect of the present invention there is provided a method manufacturing a packaged catheter assembly comprising the steps of providing a pouch comprising an absorbent material, and arranging a catheter within the pouch.

The method of the twentieth aspect of the invention may be a method of manufacturing the catheter assembly of the seventh aspect of the invention, which, of course, may include any optional feature outlined above.

According to a twenty-first aspect of the present invention there is provided a method of forming a packaged catheter assembly comprising the steps of providing a catheter and a sheet of material, folding the sheet to form two walls, and sealing the two walls with a peripheral seal to form the pouch, wherein the peripheral seal is configured to be broken to allow the fold to be opened to a flat position.

The method of the twenty-first aspect of the invention may be a method of forming the catheter assembly of the eighth aspect of the invention, which, of course, may include any optional feature outlined above.

The method may comprise arranging the contents of the pouch on the inside of one of the walls (e.g. a first wall). The method may then comprise folding the other (second) wall over the first wall and sealing the two walls with the peripheral seal. Thus, the packaged catheter assembly can be conveniently and quickly manufactured.

According to a twenty-second aspect of the present invention there is provided a method of manufacturing a packaged catheter assembly comprising providing two walls to form a pouch, arranging a catheter within the pouch, and forming a peripheral seal between the two walls to seal the pouch, wherein each wall comprises a tab extending from an edge of the pouch and the tabs are configured to be separated in use to create an opening in the peripheral seal.

The method of the twenty-second aspect of the invention may be a method of manufacturing the catheter assembly of the ninth aspect of the invention, which, of course, may include any optional feature outlined above.

The method may comprise providing a gripping hole in each tab. The method may comprise cutting or punching a gripping hole in each tab. Thus, the gripping hole can be conveniently created in the tab to make it easier to grasp.

The method may comprise folding the tabs against the pouch. The method may comprise folding the tabs against the same side of the pouch, for example the front wall of the pouch. Thus, the form factor of the pouch is reduced and the tabs are less likely to be damaged by being caught on other objects.

According to a twenty-third aspect of the present invention, there is provided a method of manufacturing an intermittent catheter assembly comprising the steps of providing a catheter having a proximal end for insertion into the body and a distal end; and a fluid collection bag, arranging the fluid collection bag to receive fluid from the distal end of the catheter, wherein the fluid collection bag comprises a hand strap.

The method of the twenty-third aspect of the invention may be a method of manufacturing the catheter assembly of the tenth aspect of the invention, which, of course, may include any optional feature outlined above.

According to a twenty-fourth aspect of the present invention, there is provided a method of manufacturing a catheter assembly comprising the steps of providing a catheter having a proximal end for insertion into the body and a distal end, and a fluid collection bag, arranging the fluid collection bag to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration.

The method of the twenty-fourth aspect of the invention may be a method of manufacturing the catheter assembly of the eleventh aspect of the invention, which, of course, may include any optional feature outlined above.

According to a twenty-fifth aspect of the present invention there is provided a method of manufacturing a catheter assembly comprising the steps of providing a catheter having a proximal end for insertion into the body and a distal end and a fluid collection bag, providing a valve assembly and arranging it between the catheter and fluid collection bag such that the fluid collection bag may receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag.

The method of the twenty-fifth aspect of the invention may be a method of manufacturing the catheter assembly of the first aspect of the invention, which, of course, may include any optional feature outlined above.

The method may comprise providing the valve assembly in the first configuration. Thus, the valve assembly may allow the user to fill the fluid collection bag and use the catheter without first needing to change the configuration of the valve assembly.

According to a twenty-sixth aspect of the present invention there is provided a method of manufacturing a catheter assembly comprising the steps of providing a catheter having a proximal end for insertion into the body and a distal end and forming a fluid collection bag, the fluid collection bag being formed by providing two panels, creating a peripheral bond between the two panels, and providing an inlet in the fluid collection bag to receive fluid from the catheter, wherein: the two panels are configured to form a base of the fluid collection bag distal from the inlet; and the fluid collection bag is configured to stand up on the base.

The method of the twenty-sixth aspect of the invention may be a method of manufacturing the catheter assembly of the second aspect of the invention, which, of course, may include any optional feature outlined above.

The method may comprise providing a base panel. The method may comprise joining the base panel to the front and/or rear panels of the fluid collection bag. The method may comprise folding the base panel. The base panel may be folded along a line parallel to a base edge of the front and/or rear panels.

The method may comprise providing two side panels. The method may comprise joining each side panel to the front and/or rear panels of the fluid collection bag. The method may comprise joining each side panel to the base panel. The method may comprise folding each side panel. Thus, the interior volume of the fluid collection bag may be further increased.

According to an twenty-seventh aspect of the present invention there is provided a method of wetting a catheter having a proximal end for insertion into the body and a distal end, the method comprising the steps of releasing wetting fluid from a fluid reservoir at the proximal end of the catheter, into a sleeve that encloses the catheter, allowing the wetting fluid to flow through the sleeve from the proximal end to the distal end, and collecting excess wetting fluid in a fluid collection bag configured to receive fluid from a distal end of the catheter and the sleeve.

The method of the twenty-seventh aspect of the invention may be a method of wetting the catheter of the catheter assembly of the first aspect of the invention, which, of course, may include any optional feature outlined above and may be manufactured according to the fourteenth aspect of the invention.

The method may comprise introducing the catheter by its proximal end into the urethra. The method may comprise allowing fluid to pass from the body and into the fluid collection bag via the catheter and/or the sleeve. Consequently, the method may be a method of using a catheter assembly.

According to a twenty-eighth aspect of the present invention, there is provided a method of draining a fluid collection bag, the fluid collection bag comprising an inlet and a sealed outlet next to one another, the outlet comprising a pull ring, the method comprising folding the inlet to form an edge of the bag with the pull ring projecting beyond the edge, moving the pull ring in a direction away from the inlet to form an opening in the bag, and emptying the contents of the bag out through the opening.

The method of the twenty-eighth aspect of the invention may be a method of draining the fluid collection bag of the second aspect of the invention, which, of course, may include any optional feature outlined above and may be manufactured according to the fifteenth aspect of the invention.

The method may comprise forming an opening extending from at least one point of weakness disposed on the outlet. The method may comprise forming an opening along a weakened region of the outlet. Thus, the opening is easily formed in a controlled manner.

The method may comprise rotating the bag such that the outlet is at a point above the rest of the bag, and then forming an opening in the bag. Thus, fluid in the bag is not near the outlet as it is opened which reduces the likelihood of spillage.

The method may comprise rotating the bag such that the outlet is at a point below the rest of the bag after forming an opening in the bag. Thus, fluid may flow out of the bag and the bag may be efficiently drained.

The method may comprise rotating the bag by grasping one or more finger holes disposed at the edges of the bag. The method may comprise grasping a finger hole disposed on an edge of the bag distal from the outlet. Thus, the bag can be rotated in a controlled manner using the finger holes and while reducing the risk of spillages from the outlet.

The method may comprise providing a catheter and introducing the catheter by its proximal end into the urethra. The method may comprise allowing fluid to pass from the body and into the fluid collection bag via the inlet and catheter. Fluid may pass from the catheter into the bag before an opening is formed in the bag with the outlet. Consequently, the method may be a method of using a catheter assembly.

According to a twenty-ninth aspect of the present invention, there is provided a method of unpacking a packaged catheter assembly, the packaged catheter assembly comprising a fluid reservoir, a catheter, and a pouch containing the fluid reservoir and catheter, wherein the catheter comprises a proximal end for insertion into the body, a distal end, and a sleeve extending from the proximal end to the distal end, wherein an end of the sleeve comprises a reservoir adapter configured to engage the fluid reservoir, the method comprising the steps of releasing wetting fluid from the fluid reservoir into the sleeve, and detaching the fluid reservoir to permit the catheter to pass through the reservoir adapter.

The method of the twenty-ninth aspect of the invention may be a method of wetting a catheter from the packaged catheter assembly of the third aspect of the invention, which, of course, may include any optional feature outlined above and may be manufactured according to the sixteenth aspect of the invention.

The method may comprise forming an opening in the pouch. The step of releasing wetting fluid may comprise rupturing the fluid reservoir, for example by pressing the activation or wetting marker. The method may comprise pressing the fluid reservoir against or towards the sleeve to release wetting fluid from the fluid reservoir. The method may comprise pressing the fluid reservoir against or towards the proximal end of the catheter to release wetting fluid from the fluid reservoir. The method may comprise grasping the reservoir adapter and pressing the fluid reservoir against/towards the reservoir adapter to allow fluid to be released from the fluid reservoir. The method may comprise allowing fluid to flow through the reservoir adapter. Thus, the fluid reservoir may be easily activated to release wetting fluid into the sleeve.

Accordingly in one embodiment, there is provided a method of wetting a catheter having a proximal end for insertion into the body and a distal end, the method comprising the steps of releasing wetting fluid from a fluid reservoir at the proximal end of the catheter, into a sleeve that encloses the catheter, allowing the wetting fluid to flow through the sleeve from the proximal end to the distal end, and collecting excess wetting fluid in a fluid collection bag configured to receive fluid from a distal end of the catheter and the sleeve wherein the method comprises pressing the fluid reservoir against or towards the proximal end of the catheter to release wetting fluid from the fluid reservoir.

The fluid reservoir may be detached from the reservoir adapter after the release of wetting fluid from the fluid reservoir. Thus, the catheter is adequately wetted before the fluid reservoir is removed.

The method may comprise removing the fluid reservoir from the pouch. The method may comprise detaching the reservoir adapter from the fluid reservoir after removing the fluid reservoir from the pouch. Thus, the method may comprise removing the fluid reservoir from the sleeve, preferably after release of wetting fluid.

Accordingly in one embodiment, there is provided a method of wetting a catheter having a proximal end for insertion into the body and a distal end, the method comprising the steps of releasing wetting fluid from a fluid reservoir at the proximal end of the catheter, into a sleeve that encloses the catheter, allowing the wetting fluid to flow through the sleeve from the proximal end to the distal end, and collecting excess wetting fluid in a fluid collection bag configured to receive fluid from a distal end of the catheter and the sleeve wherein the method comprises removing the fluid reservoir from the sleeve.

The method may comprise detaching the reservoir adapter from the fluid reservoir by removing the reservoir adapter from the pouch.

The method may comprise withdrawing the catheter from the sleeve. Only part of the catheter, preferably including the proximal end of the catheter, may be withdrawn from the sleeve. The method may comprise passing the catheter through the reservoir adapter. The method may comprise bunching the sleeve. The catheter may be withdrawn from the sleeve after it has been wetted. The catheter may be withdrawn from the sleeve after the fluid reservoir has been removed from the sleeve. Thus, the fluid reservoir protects the catheter before use, and the catheter is easily withdrawn through the reservoir adapter.

The method may comprise introducing the catheter by its proximal end into the urethra. The method may comprise allowing fluid to pass from the body and into the fluid collection bag via the catheter and/or the sleeve. Consequently, the method may be a method of using a catheter assembly.

According to an thirtieth aspect of the present invention there is provided a method of wetting a catheter of a catheter assembly, the catheter assembly comprising the catheter having a proximal end for insertion into the body, a distal end and a sleeve extending from the proximal end to the distal end, and a jacket surrounding a fluid reservoir, the method comprising releasing wetting fluid from the fluid reservoir into the jacket and directing wetting fluid from the jacket into the sleeve at the proximal end of the catheter.

The method of the thirtieth aspect of the invention may be a method of unpackaging a packaged catheter assembly of the fourth aspect of the invention, which, of course, may include any optional feature outlined above and may be manufactured according to the seventeenth aspect of the invention.

The method may comprise activating the fluid reservoir to release wetting fluid via an interaction with the jacket. The method may comprise removing the jacket and fluid reservoir from the sleeve.

The method may comprise removing the jacket from the pouch. The method may comprise detaching the reservoir adapter from the jacket after removing the fluid reservoir from the pouch.

The method may comprise detaching the reservoir adapter from the jacket by removing the reservoir adapter from the pouch.

The method may comprise introducing the catheter by its proximal end into the urethra. The method may comprise allowing fluid to pass out of the body via the catheter. Consequently, the method may be a method of using a packaged catheter assembly.

According to a thirty-first aspect of the present invention there is provided a method of unpackaging a packaged catheter assembly, the packaged catheter assembly comprising a catheter, a fluid collection bag configured to receive fluid from the catheter in use, and a pouch containing the catheter and fluid collection bag, the method comprising using an interaction region to form an opening in the pouch to allow the catheter and fluid collection bag to be removed from the pouch, wherein the fluid collection bag is arranged adjacent to the opening.

The method of the thirty-first aspect of the invention may be a method of unpackaging a packaged catheter assembly according to the fifth aspect of the invention and may, of course, include any optional feature outlined above and the packaged catheter assembly may be manufactured according to the eighteenth aspect of the invention.

The method may comprise withdrawing the fluid collection bag from the pouch before the catheter. The method may comprise withdrawing the catheter from the pouch using the fluid collection bag.

The method may comprise unfolding the fluid collection bag. The method may comprise handling the fluid collection bag via one or more projections and/or one or more finger holes disposed on the fluid collection bag.

The method may comprise measuring the fluid contained in the fluid collection bag via a fill level indicator disposed on the fluid collection bag.

The method may comprise introducing the catheter by its proximal end into the urethra. The method may comprise allowing fluid to pass out of the body via the catheter. Consequently, the method may be a method of using a packaged catheter assembly.

According to a thirty-second aspect of the present invention there is provided a method of unpackaging a packaged catheter assembly, the packaged catheter assembly comprising a pouch and a catheter arranged within the pouch, wherein the catheter is arranged within the pouch in a shape having two opposing turns of at least 180 degrees along the catheter's length, the method comprising forming an opening in the pouch and withdrawing the catheter from the pouch through the opening.

The method of the thirty-second aspect of the invention may be a method of unpackaging a packaged catheter assembly according to the sixth aspect of the invention and may, of course, include any optional feature outlined above and the packaged catheter assembly may be manufactured according to the nineteenth aspect of the invention.

The method may comprise withdrawing the catheter from the pouch by holding it at one end. The method may comprise withdrawing the catheter from the pouch by holding it at a position corresponding to one of the turns. The method may comprise withdrawing the catheter from the pouch by holding it in two places: at one end and at a position corresponding to the second turn, optionally wherein the end and second turn are separated by the other (first) turn. This allows the catheter to be withdrawn in one motion without causing the catheter to be pulled out by one end alone which may damage the catheter by causing the radius of curvature at one of the turns to become so small that the catheter kinks.

The method may comprise unravelling the two opposing turns. The method may comprise straightening the catheter. The method may comprise wetting the catheter, for example as described in relation to the twelfth or fourteenth aspect of the invention. Preferably, the two opposing turns may be unravelled/the catheter straightened before the catheter is wetted. The combination of these aspects is particularly advantageous as the orientation/curvature of the catheter is optimised to ensure wetting fluid can easily flow through the sleeve and wet the full length of the catheter prior to use.

The method may comprise introducing the catheter by its proximal end into the urethra. The method may comprise allowing fluid to pass out of the body via the catheter. Consequently, the method may be a method of using a packaged catheter assembly.

According to a thirty-third aspect of the present invention there is provided a method of unpackaging a packaged catheter assembly, the packaged catheter assembly comprising a pouch and a catheter, wherein the pouch is configured to contain the catheter within it and wherein the pouch comprises an absorbent material, the method comprising removing the catheter from the pouch.

The method of the thirty-third aspect of the invention may be a method of unpackaging a catheter assembly according to the seventh aspect of the invention and may, of course, include any optional feature outlined above and the catheter assembly may be manufactured according to the twentieth aspect of the invention.

The method may comprise wetting the catheter and absorbing any excess wetting fluid with the absorbent material.

The method may comprise introducing the catheter by its proximal end into the urethra. The method may comprise allowing fluid to pass out of the body via the catheter. Consequently, the method may be a method of using a packaged catheter assembly.

The method may comprise absorbing liquid that has passed through the catheter. Thus, the volume of free liquid is reduced by the absorbent material and this can make transportation and disposal of the liquid easier and more convenient for the user.

The method comprising absorbing liquid present on the outside of the catheter. This may preferably be done after use of the catheter. The method may comprise placing the catheter within the pouch. The method may comprise closing the pouch. Thus, any excess liquids are absorbed by the absorbent material and retained within the pouch after use of the catheter.

According to a thirty-fourth aspect of the present invention there is provided a method of unpackaging a packaged catheter assembly, the packaged catheter assembly comprising a pouch and a catheter, the method comprising the steps of breaking a peripheral seal of the pouch, and opening a fold in the pouch to a flat position. Preferably the step of unfolding the pouch involves unfolding the pouch about a fold-line which joins a first and second wall of the pouch.

The method of the thirty-fourth aspect of the invention may be a method of unpackaging a packaged catheter assembly of the eighth aspect of the invention, which, of course, may include any optional feature outlined above and may be manufactured according to the twenty-first aspect of the invention.

The method may comprise applying a threshold force to separate the first and second walls. This may allow them to be separated in a controlled manner, and the walls may then remain separated and flat as described above in relation to the eighth aspect of the invention.

The method may comprise removing the catheter from the pouch after opening the fold in the pouch. The method may comprise introducing the catheter by its proximal end into the urethra. The method may comprise allowing fluid to pass out of the body via the catheter. Consequently, the method may be a method of using a packaged catheter assembly.

The method may comprise bringing the walls of the pouch together to close the pouch. The method may comprise re-sealing the peripheral seal of the pouch. The method may comprise closing/re-sealing the pouch with the catheter within it. The steps of closing/re-sealing the pouch may be performed after use of the catheter. Thus, the pouch can be used to cleanly store the catheter after use and prior to disposal.

According to a thirty-fifth aspect of the present invention there is provided a method of unpackaging a packaged catheter assembly, the packaged catheter assembly comprising a catheter and a pouch having two walls and a peripheral seal between the two walls, wherein the pouch is configured to contain the catheter within it, each wall comprises a tab extending from an edge of the pouch, and the tabs are configured to be separated in use to create an opening in the peripheral seal, the method comprising separating the tabs to form an opening in the peripheral seal.

The method of the thirty-fifth aspect of the invention may be a method of unpackaging a catheter assembly according to the ninth aspect of the invention and may, of course, include any optional feature outlined above and the catheter assembly may be manufactured according to the twenty-second aspect of the invention.

The method may comprise unfolding the tabs. The tabs may be unfolded prior to separation of the tabs. Thus, the tabs are more easily accessible and opening of the pouch is easier.

The method may comprise handling the pouch by grasping either one, or both, of the tabs. The pouch may be handled by grasping a, or each, tab either before, after, or before and after use of the catheter. Thus, the pouch can be handled/moved by the user as required without needing to touch the inside of the pouch which they may wish to keep clean or avoid as it is dirty.

The method may comprise removing the catheter from the pouch after opening the pouch. The method may comprise introducing the catheter by its proximal end into the urethra. The method may comprise allowing fluid to pass out of the body via the catheter. Consequently, the method may be a method of using a packaged catheter assembly.

The method may comprise closing/re-sealing the pouch as described in relation to the thirtieth aspect above. The step of closing/re-sealing the pouch may comprise grasping each tab and bringing them together. Thus, the tabs can be used to avoid contact with the walls of the pouch which may have become dirty during use of the catheter.

According to a thirty-sixth aspect of the present invention, there is provided a method of unpackaging a packaged intermittent catheter assembly, the packaged catheter assembly comprising a catheter having a proximal end for insertion into the body and a distal end; and a fluid collection bag arranged to receive fluid from the distal end of the catheter, wherein the fluid collection bag comprises a hand strap, further wherein the catheter and fluid collection bag are arranged within a pouch.

The method of the thirty-sixth aspect of the invention may be a method of unpackaging a packaged catheter assembly of the tenth aspect of the invention, which, of course, may include any optional feature outlined above and may be manufactured according to the twenty-third aspect of the invention.

According to a thirty-seventh aspect of the present invention, there is provided a method of unpackaging a packaged catheter assembly, the packaged catheter assembly comprising a catheter assembly comprising the steps of providing a catheter having a proximal end for insertion into the body and a distal end, and a fluid collection bag, arranging the fluid collection bag to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration, wherein the catheter and the fluid collection bag are arranged within a pouch.

The method of the thirty-seventh aspect of the invention may be a method of unpackaging a packaged catheter assembly of the eleventh aspect of the invention, which, of course, may include any optional feature outlined above and may be manufactured according to the twenty-fourth aspect of the invention.

According to a thirty-eighth aspect of the present invention there is provided a method of draining a fluid collection bag of a catheter assembly, the catheter assembly comprising a catheter having a proximal end for insertion into the body and a distal end, a valve assembly, and a fluid collection bag arranged to receive fluid from the distal end of the catheter via the valve assembly, the method comprising switching the valve assembly from a first configuration in which the valve assembly inhibits fluid flow in a direction from the fluid collection bag towards the catheter and permits fluid flow in a direction from the catheter towards the fluid collection bag to a second configuration in which the valve assembly permits fluid flow in a direction from the fluid collection bag towards the catheter and inhibits fluid flow in a direction from the catheter towards the fluid collection bag, and allowing the fluid collection bag to drain through the valve assembly.

The method of thirty-eighth aspect of the present invention may be a method of preparing the catheter assembly of the twelfth aspect of the invention, which, of course, may include any optional feature outlined above and may be manufactured according to the twenty-fifth aspect of the invention.

The method may comprise draining the fluid collection bag through the catheter. The method may comprise draining the fluid collection bag through a sleeve configured to enclose the catheter. Thus, the sleeve and catheter may be used to direct the flow of liquid out of the fluid collection bag.

The method may comprise introducing the catheter by its proximal end into the urethra before draining the fluid collection bag. The method may comprise allowing fluid to pass from the body and into the fluid collection bag via the catheter before draining the fluid collection bag. Consequently, the method may be a method of using a catheter assembly.

According to a thirty-ninth aspect of the present invention there is provided a method of preparing a catheter assembly for use, the catheter assembly comprising a catheter and the fluid collection bag, the fluid collection bag comprising two panels, a peripheral bond between the two panels, and an inlet to receive fluid from the catheter, the method comprising using the two panels to form a base of the fluid collection bag distal from the inlet; and standing the fluid collection bag up on the base.

The method of thirty-ninth aspect of the present invention may be a method of preparing the catheter assembly of the thirteenth aspect of the invention, which, of course, may include any optional feature outlined above and may be manufactured according to the twenty-sixth aspect of the invention.

The method may comprise separating the two panels (i.e. the front and rear panels) to form the base. The method may comprise unfolding a base panel of the fluid collection bag. The method may comprise introducing liquid into the fluid collection bag. The method may comprise introducing liquid into the fluid collection bag to automatically form the base in the fluid collection bag.

The method may comprise introducing the catheter by its proximal end into the urethra. The method may comprise allowing fluid to pass from the body and into the fluid collection bag via the catheter. Consequently, the method may be a method of using a catheter assembly.

The methods of the fourteenth to thirty-ninth aspects of the present invention may of course individually include any one or more of the features, optional or otherwise, of one another and further may include any one or more of the features, optional or otherwise, of the first to thirteenth aspects of the present invention.

In any aspect the catheter is preferably a urinary catheter; and/or is an intermittent catheter. Thus, the features of aspects of the present invention allow intermittent male urinary catheters to be adequately wetted prior to use which can be more difficult than for other types of catheters which are generally shorter.

### DETAILED DESCRIPTION OF THE INVENTION

In order that the invention may be more clearly understood one or more embodiments thereof will now be described, by way of example only, with reference to the accompanying drawings, of which:

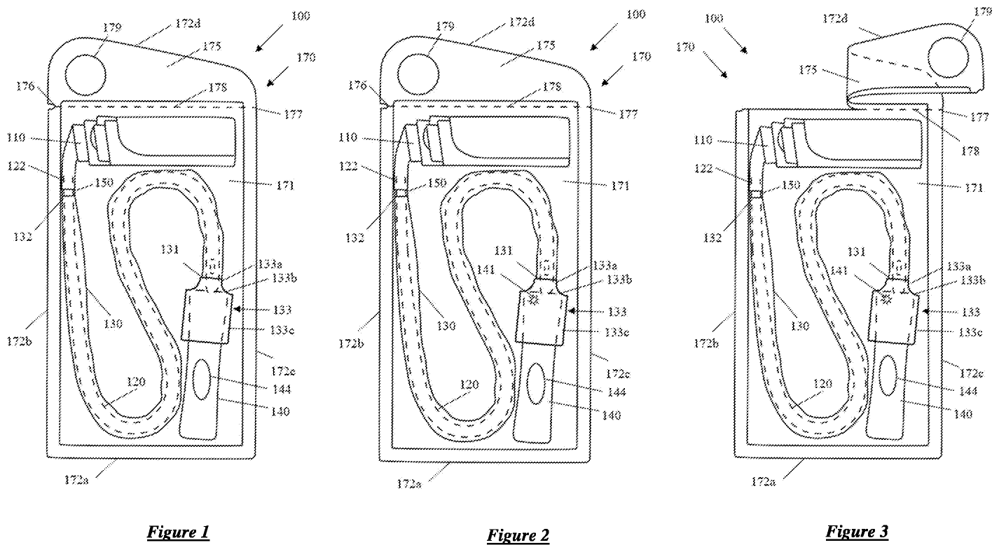

FIG. 1 is a cut-away front view of a first embodiment of a packaged catheter assembly;

FIG. 2 is a cut-away front view of the packaged catheter assembly of FIG. 1 where wetting fluid from the fluid reservoir has been released into the sleeve;

FIG. 3 is a cut-away front view of the packaged catheter assembly of FIG. 1 during forming an opening in the pouch;

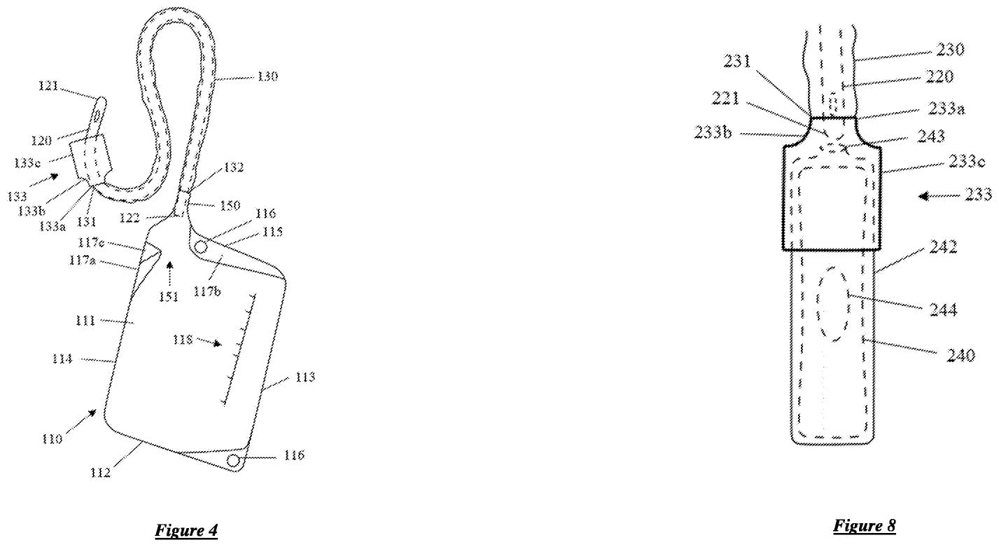

FIG. 4 is a cut-away front view of the catheter assembly of FIG. 1 where the closed catheter assembly has been removed from the pouch; and

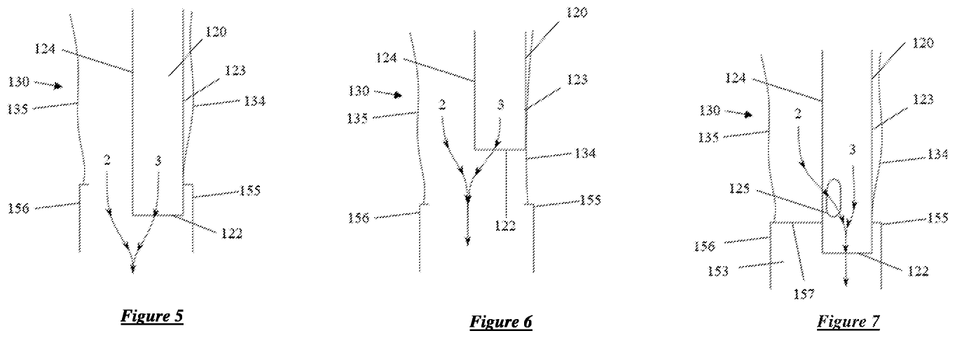

FIG. 5 is a cut-away view of a first example of the inlet of the closed catheter assembly of FIG. 1;

FIG. 6 is a cut-away view of a second example of the inlet of the closed catheter assembly of FIG. 1;

FIG. 7 is a cut-away view of a third example of the inlet of the closed catheter assembly of FIG. 1;

FIG. 8 is a front view of the reservoir adapter of a second embodiment of a packaged catheter assembly;

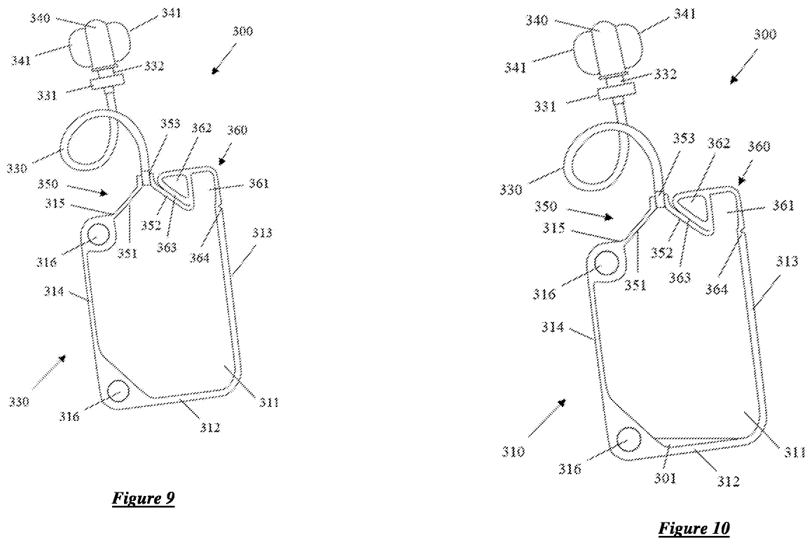

FIG. 9 is a front view of a third embodiment of a catheter assembly;

FIG. 10 is a front view of the catheter assembly of FIG. 9 where wetting fluid from the fluid reservoir has wetted the catheter;

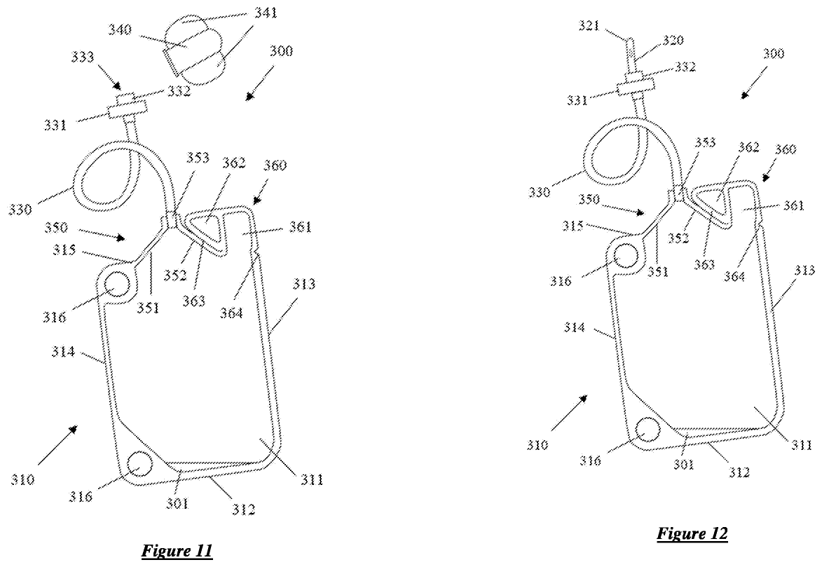

FIG. 11 is a front view of the catheter assembly of FIG. 9 where the fluid reservoir has been removed from the catheter;

FIG. 12 is a front view of the catheter assembly of FIG. 9 where the catheter the sleeve is only covering part of the length of the catheter;

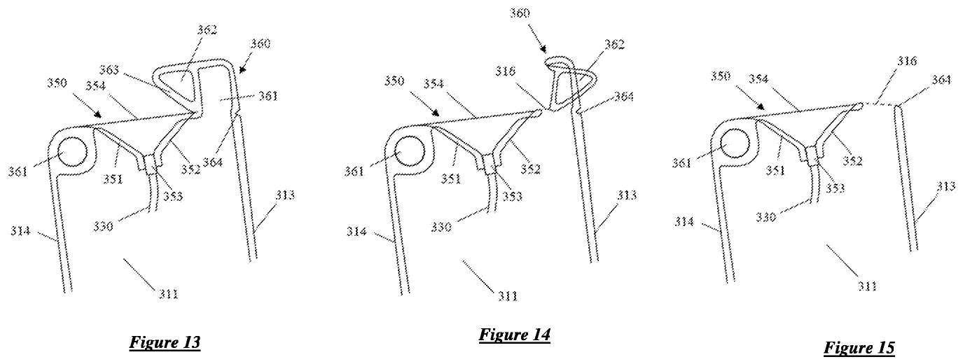

FIG. 13 is a front view a fluid collection bag of the catheter assembly of FIG. 9 where the neck has been folded;

FIG. 14 is a front view a fluid collection bag of the catheter assembly of FIG. 9 where the outlet has been partially opened;

FIG. 15 is a front view of the fluid collection bag of FIG. 9 where the outlet has been fully opened;

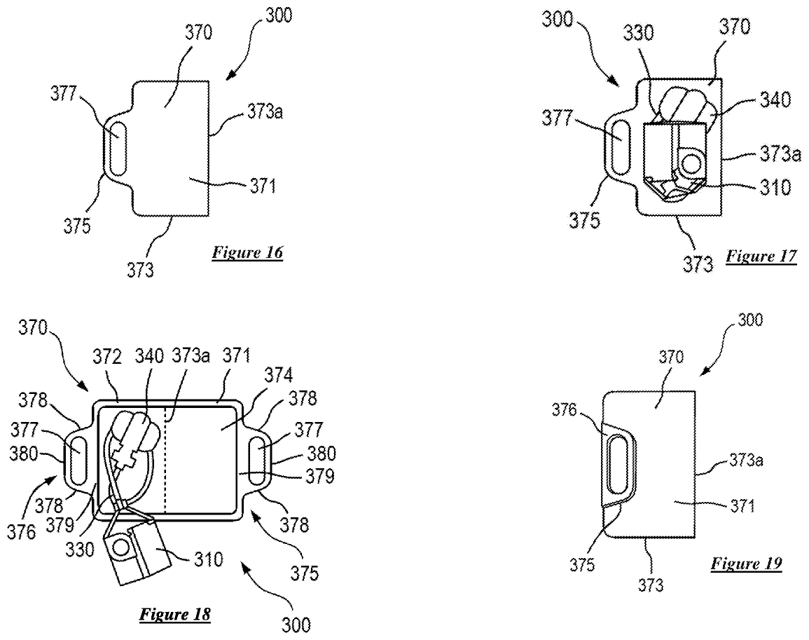

FIG. 16 is a front view of a pouch of the catheter assembly of FIG. 9;

FIG. 17 is a cut-away view of the interior of the pouch of the catheter assembly of FIG. 9;

FIG. 18 is a front view of the catheter assembly of FIG. 9 where the pouch has been opened;

FIG. 19 is a front view of the catheter assembly of FIG. 9 where the pouch has been closed;

FIG. 20 is a cut-away view of a first example of the inlet of the catheter assembly of FIG. 9;

FIG. 21 is a cut-away view of a second example of the inlet of the catheter assembly of FIG. 9;

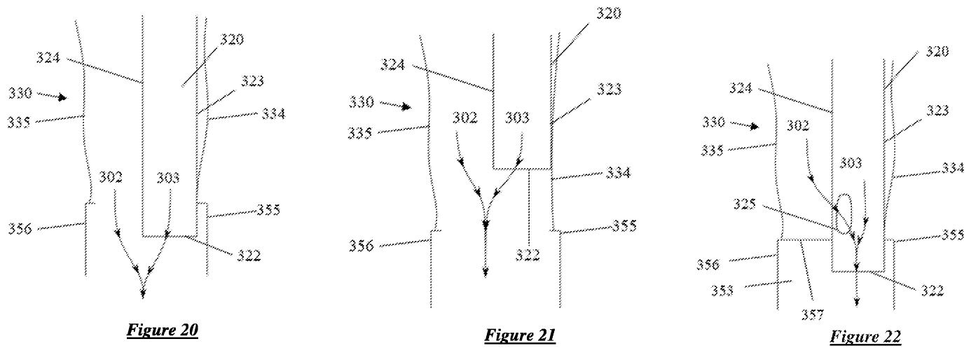

FIG. 22 is a cut-away view of a third example of the inlet of the catheter assembly of FIG. 9;

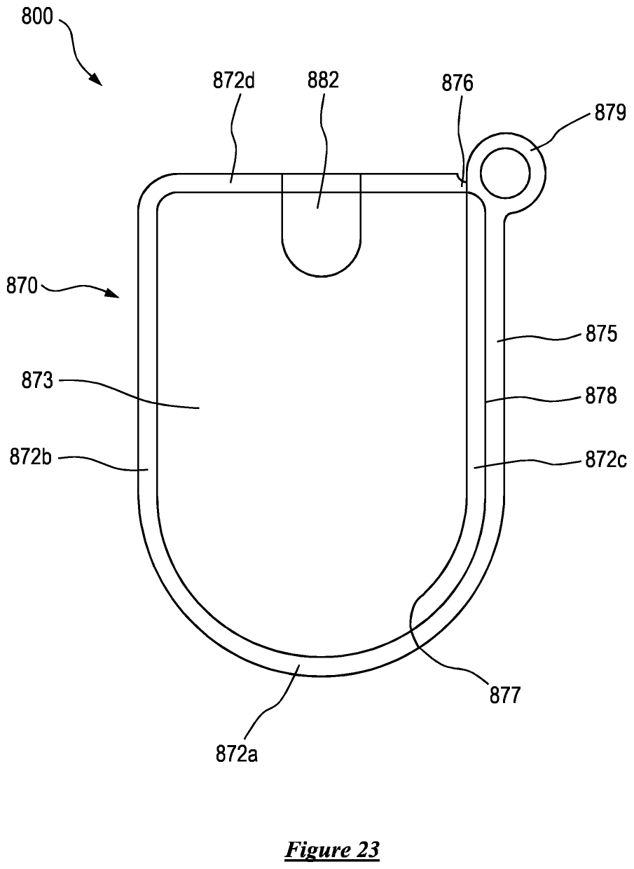

FIG. 23 is a front view of a fourth embodiment of a packaged catheter assembly;

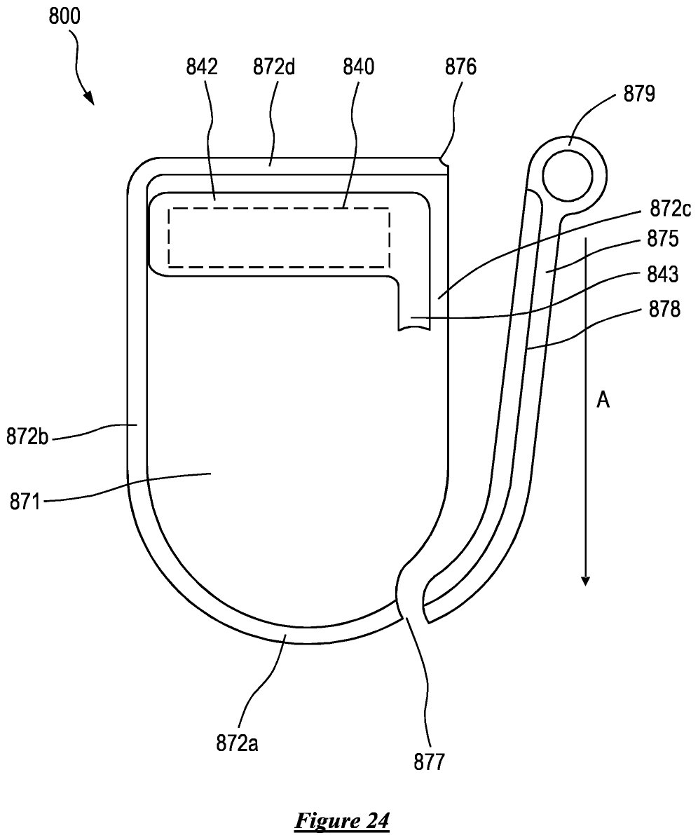

FIG. 24 is a cut-away front view of the packaged catheter assembly of FIG. 23 during forming an opening in the pouch;

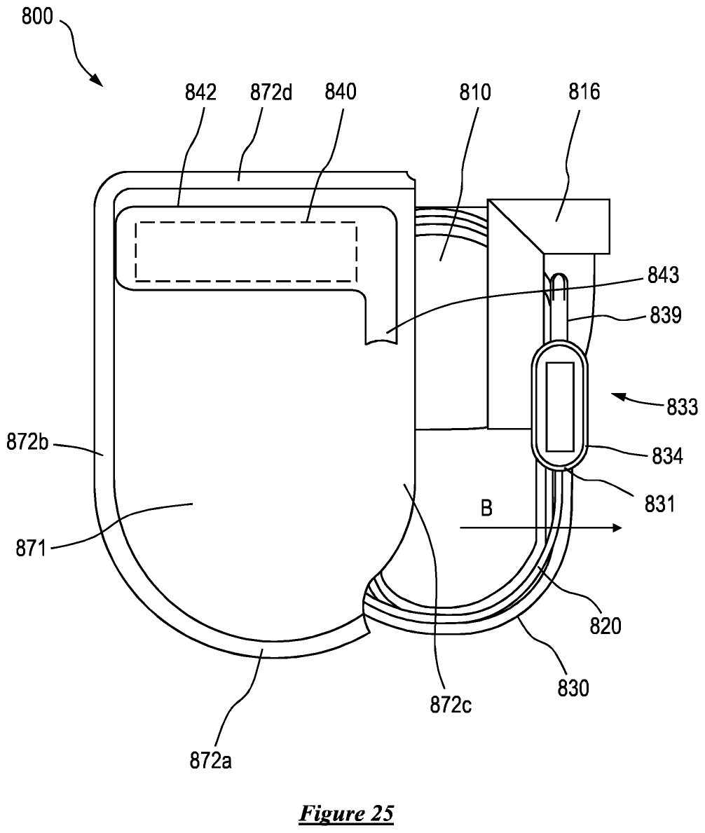

FIG. 25 is a cut-away front view of the packaged catheter assembly of FIG. 23 during withdrawal of the catheter assembly from the pouch;

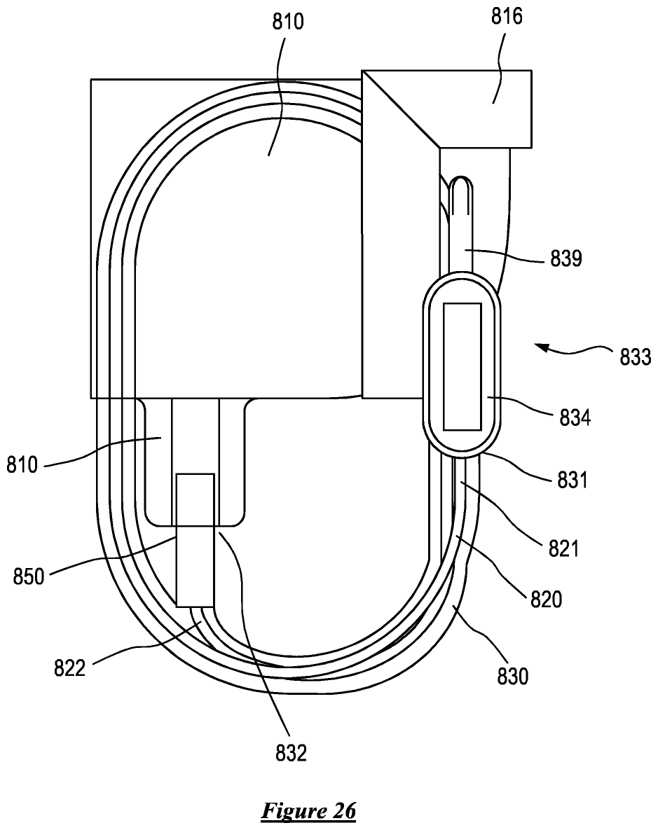

FIG. 26 is a front view of the catheter assembly of FIG. 23 where the catheter assembly has been removed from the pouch;

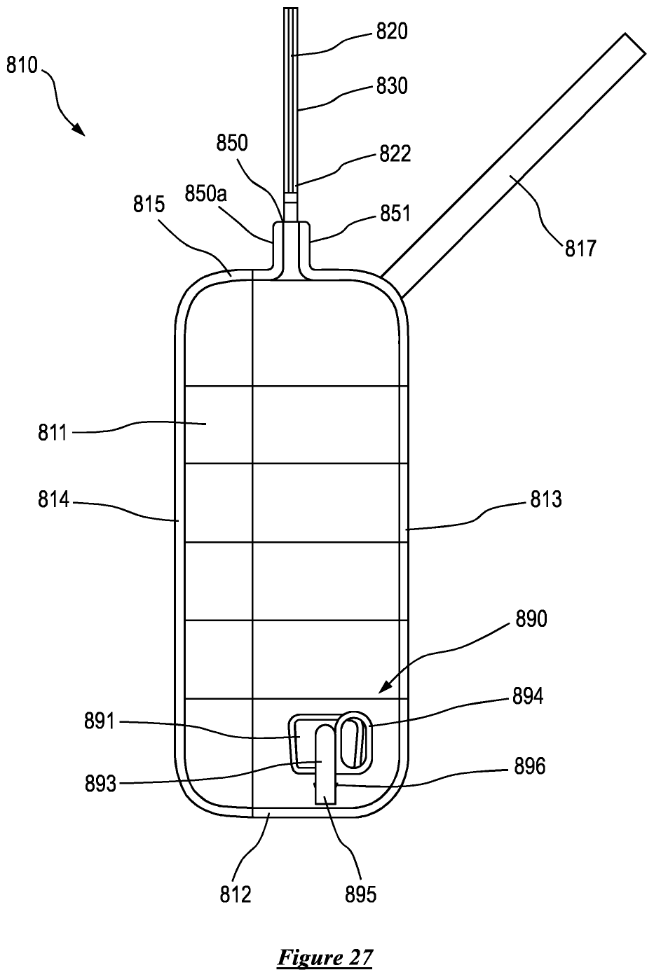

FIG. 27 is a front view of a fluid collection bag catheter assembly of FIG. 23;

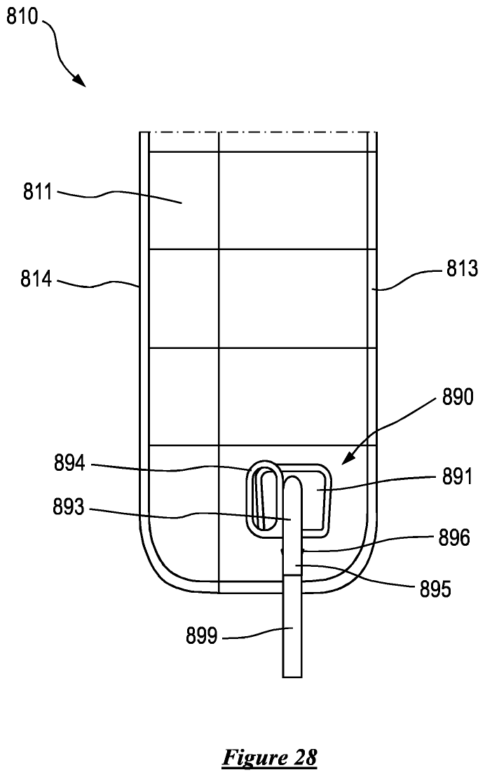

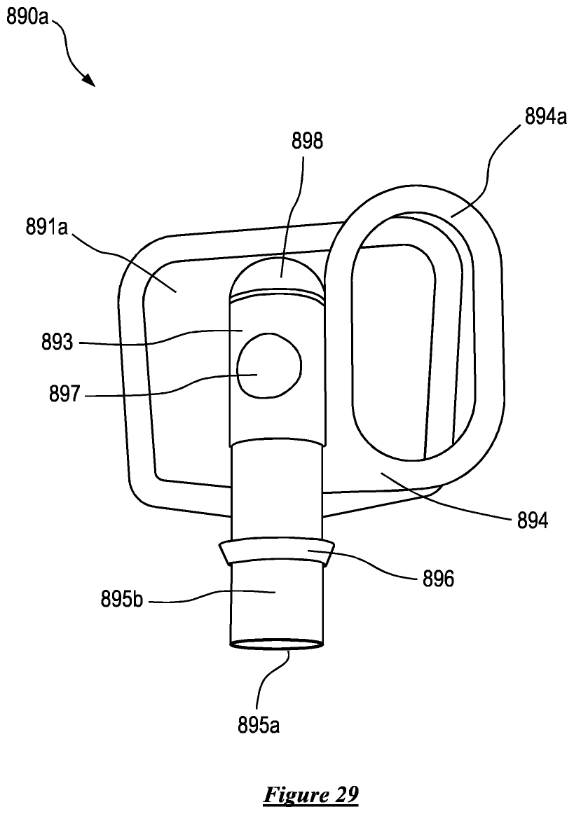

FIG. 28 is a partial front view of a fluid collection bag catheter assembly of FIG. 23;

FIG. 29 is a front view of an outlet valve of the fluid collection bag of FIG. 27;

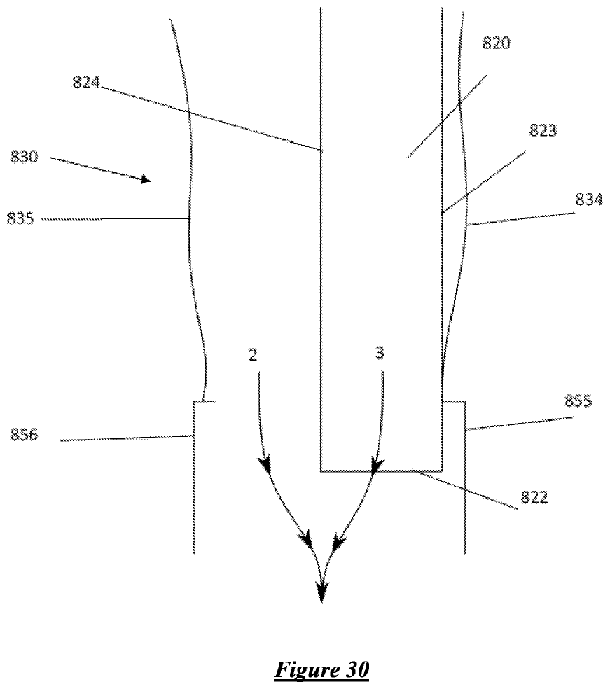

FIG. 30 is a cut-away view of a first example of the inlet of the catheter assembly of FIG. 23;

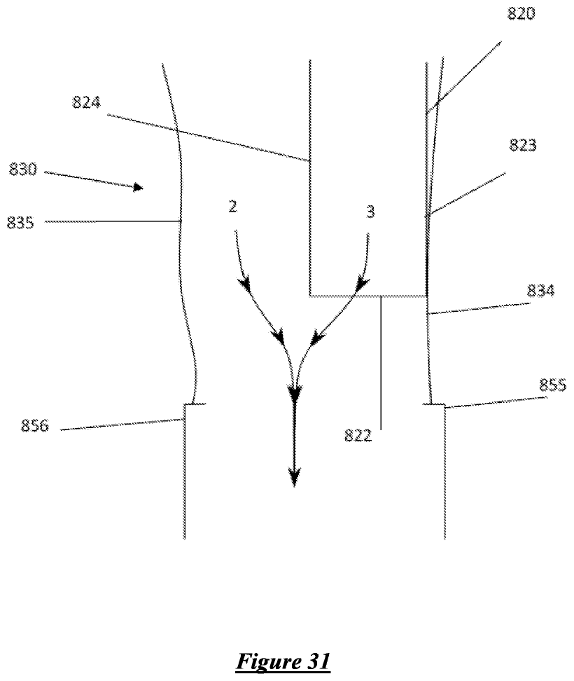

FIG. 31 is a cut-away view of a second example of the inlet of the catheter assembly of FIG. 23;

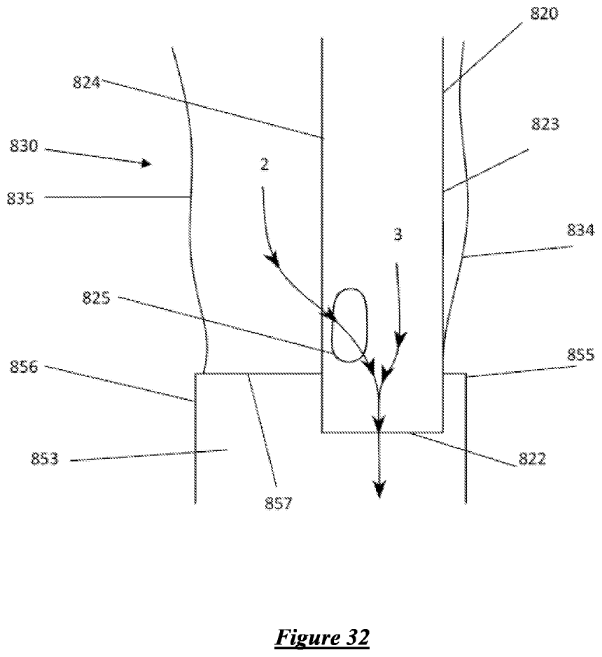

FIG. 32 is a cut-away view of a third example of the inlet of the catheter assembly of FIG. 23;

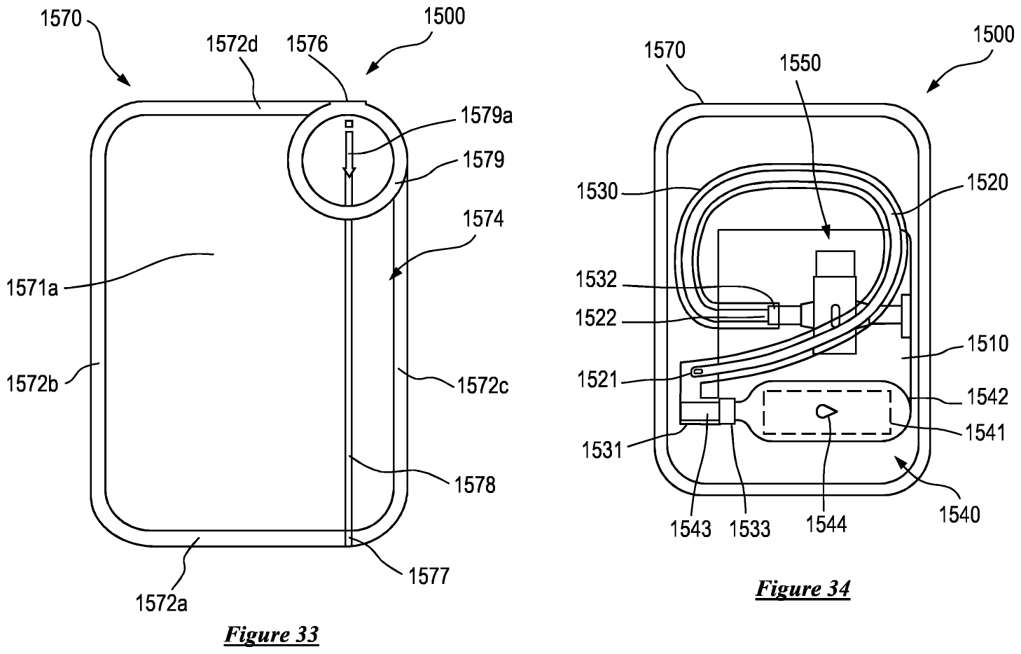

FIG. 33 is a front view of a fifth embodiment of a packaged catheter assembly where the pouch is in the closed configuration;

FIG. 34 is a front cut-away view of the packaged catheter assembly of FIG. 33;

FIG. 35 is a front view of the packaged catheter assembly of FIG. 33 where the pouch is in the open configuration and the catheter assembly contained within the pouch is being removed from the pouch;

FIG. 36 is a front view of the catheter assembly from the packaged catheter assembly of FIG. 33;

FIG. 37 is a schematic diagram of showing the operation of the valve assembly of the packaged catheter assembly of FIG. 33;

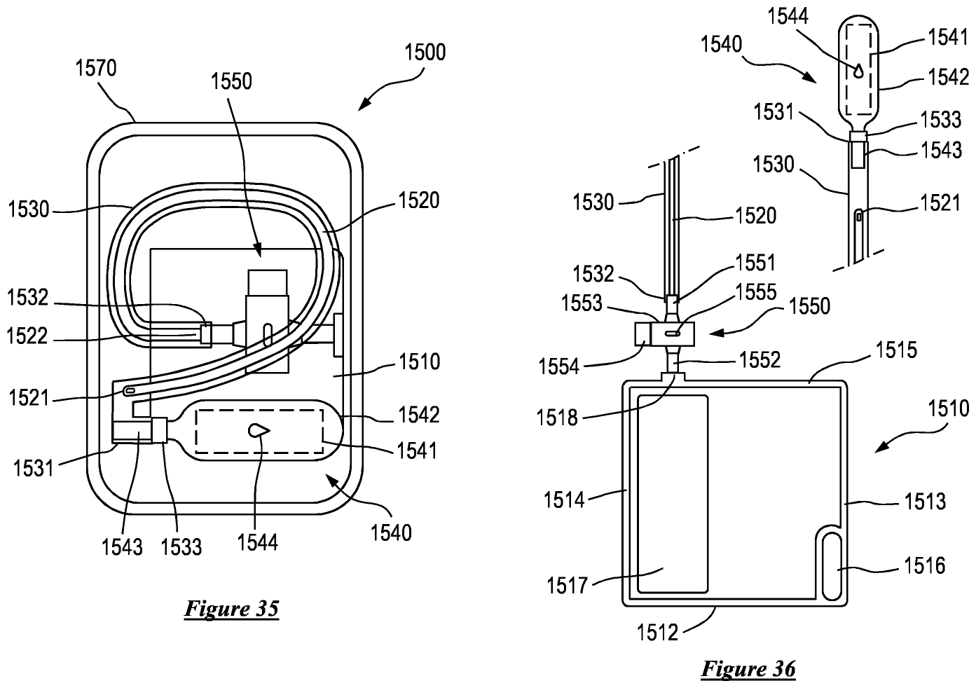

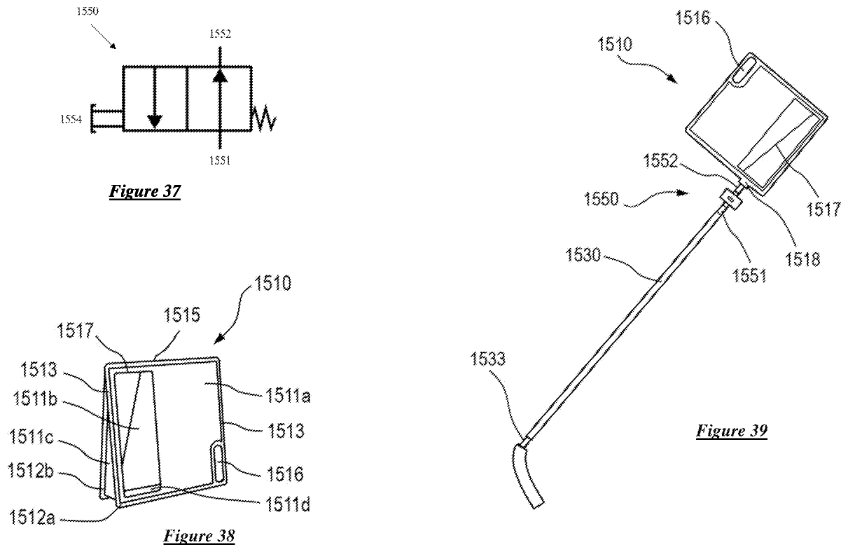

FIG. 38 is a perspective view of the fluid collection bag of the packaged catheter assembly of FIG. 33;

FIG. 39 is a front view of liquid being drained from the fluid collection bag of the packaged catheter assembly of FIG. 33;

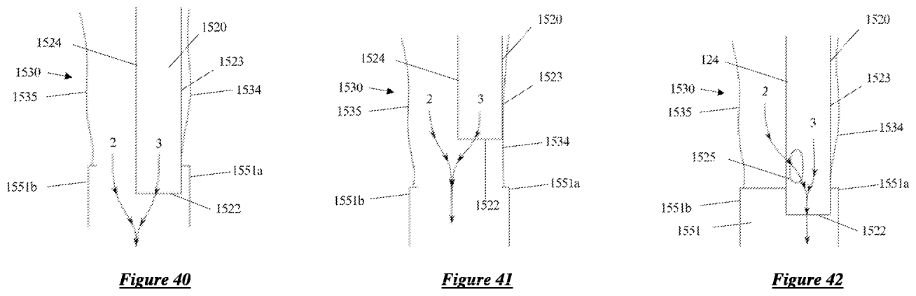

FIG. 40 is a cut-away view of a first example of the inlet of the catheter assembly of FIG. 33;

FIG. 41 is a cut-away view of a second example of the inlet of the catheter assembly of FIG. 33;

FIG. 42 is a cut-away view of a third example of the inlet of the catheter assembly of FIG. 33;

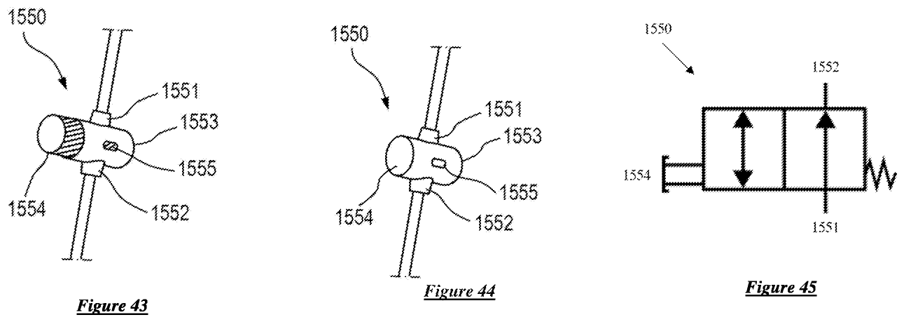

FIG. 43 is a perspective view of a variation on the valve assembly of the packaged catheter assembly of FIG. 33 in the first configuration;

FIG. 44 is a perspective view of the valve assembly of FIG. 43 in the second configuration; and

FIG. 45 is a schematic diagram of showing the operation of the valve assembly of FIG. 43.

In the Figures, as is conventional, broken lines show hidden features.

Referring to FIGS. 1-4, an embodiment of a packaged catheter assembly 100 is shown.

The packaged catheter assembly 100 comprises a pouch 170 containing a fluid collection bag 110, a catheter tube, or “catheter” 120, a sleeve 130, and a fluid reservoir 140 comprising wetting fluid. The catheter 120 has a proximal end 121 for insertion into the body and a distal end 122, and the fluid collection bag 110 is attached to the distal end 122 of the catheter 120 and is arranged to receive fluid from the distal end 122 of the catheter 120, thereby forming a so-called “closed catheter assembly”. In this embodiment, the sleeve 130 and fluid reservoir 140 are also attached to the catheter 120 and so are also considered part of the “closed catheter assembly”.

In this embodiment, the catheter 120 is a male urinary catheter made from a hydrophilic thermoplastic elastomer (TPE). The sleeve 130 of this embodiment is a thermoplastic polyurethane (TPU) or low-density polyethylene (LDPE). Obviously those skilled in the art will be able to select suitable alternative materials.

The sleeve 130 of this embodiment is sized to enclose the catheter 120 along the majority of the length of the catheter 120, and preferably the entire length of the catheter 120. The sleeve 130 comprises a proximal end 131 corresponding to the proximal end 121 of the catheter 120 and a distal end 132 corresponding to the distal end 122 of the catheter 120. The distal end 132 of the sleeve 130 is attached to an inlet 150 of the fluid collection bag 110. Therefore, in this embodiment the sleeve 130 is not separatable from the catheter 120. The proximal end 131 of the sleeve comprises a reservoir adapter 133.

In this embodiment, the reservoir adapter 133 is generally tubular with a constant wall thickness and comprises a bore through its entire length. Of course, in other embodiments the wall thickness may vary allowing the size/shape of the reservoir adapter to be varied as desired. The reservoir adapter 133 comprises a distal end 133a with annular cross-section where it is attached to the proximal end 131 of the sleeve 130. The distal end 133a comprises an internal diameter sized to allow the catheter 120 and wetting fluid to pass through the reservoir adapter 133 as described further below.

The reservoir adapter 133 is configured to releasably engage the fluid reservoir 140, and as such, a proximal end 133c of the reservoir adapter 133 distal from the sleeve 130 is configured to receive and retain the fluid reservoir 140. In this embodiment, the reservoir adapter 133 has an internal size and shape that matches the fluid reservoir 140 to allow the fluid reservoir to be push-fit into the reservoir adapter 133. This also allows the fluid reservoir 140 to be easily detached from the reservoir adapter 133 by pulling them apart. Of course, in other embodiments the fluid reservoir 140 may be received in a different way, such as through a bayonet or screw type fit, or through use of a temporary adhesive.

In this embodiment, the fluid reservoir 140 is an elongate rectangular burstable sachet of water. The fluid reservoir 140 is arranged with a first end engaged into the reservoir adapter 133. The reservoir adapter 133 extends around the first end of the fluid reservoir 140 and along the fluid reservoir 140 between 60-20% of the length of the fluid reservoir 140, for example 33%. Consequently, the proximal end 133b of the reservoir adapter 133 comprises a generally elliptical annular cross section to allow an interference fit between the fluid reservoir 140 and reservoir adapter 133.

In this embodiment, the reservoir adapter 133 also comprises a midsection 133b between the annular distal end 133a and larger elliptical proximal end 133c enclosing the fluid reservoir 140. The midsection 133b is tapered and has a convex shape to assist the user in gripping the reservoir adapter and using it as a handling element for the catheter 120 and/or sleeve 130. The midsection 133b also acts as a funnel to deliver wetting fluid from the fluid reservoir 140 to the catheter 120/sleeve 130.

In this embodiment, the fluid reservoir 140 is be configured to retain 5 to 50 ml of wetting fluid, for example 12 ml. The fluid reservoir 140 has a height equal to between 20-50% the height of the pouch 170, for example 30%, and a width between 5-25% the width of the pouch 170, for example 15%. The height and width of the pouch 170 are defined as the distance between a base 172a and an upper edge 172d of the pouch, and a left lateral edge 172b and right lateral edge 172c of the pouch 170 respectively and as described in more detail below.

The fluid reservoir 140 also comprises a wetting marker 144 configured to allow the user to identify where to activate the fluid reservoir 140 to release wetting fluid. In this embodiment, the wetting marker 144 comprises a logo/region of different colour or contrast from the rest of the fluid reservoir 140. In this embodiment, the wetting marker 144 is positioned in a region of the fluid reservoir 140 that is not enclosed by the reservoir adapter 133, this allows the user to easily identify the wetting marker 144 and activate the fluid reservoir 140. In other embodiments, the wetting marker 144 may provide any suitable visual or tactile indication to assist the user in identifying and activating the fluid reservoir 140 to release wetting fluid.

In this embodiment, the fluid reservoir 140 is configured to release wetting fluid by rupturing upon depression/compression of the fluid reservoir 140, especially at a position corresponding to the wetting marker 144. The fluid reservoir 140 is configured to rupture and create a tear 141 in the fluid reservoir 140 through which wetting fluid is released. In this embodiment, the fluid reservoir 140 is configured to create a tear 141 in locations that are enclosed by the reservoir adapter 133. Thus, the reservoir adapter 133 is therefore configured to receive wetting fluid from the fluid reservoir 140 into the sleeve 130 to wet the catheter 120. To ensure that wetting fluid released from the fluid reservoir 140 is directed into the sleeve 130, the reservoir adapter 133 and/or fluid reservoir 140 may comprise a sealing element to prevent wetting fluid from escaping into the pouch 170, for example an O-ring provided around the end of the reservoir adapter 133 that engages the fluid reservoir 140. Such an O-ring may also facilitate the push-fit engagement of the fluid reservoir 140 and reservoir adapter 133.

In other embodiments, many other types of fluid reservoir would be suitable including different shapes and sizes of reservoir, or non-burstable reservoirs and other external sources of fluid. In this embodiment, the wetting fluid is water and interacts with the hydrophilic surface of the catheter 120 to render it lubricious. In other embodiments, other wetting fluids may be used and they may be polar (e.g. water-based) or non-polar (e.g. oil-based) depending on the catheter's surface properties.

In this embodiment, the fluid reservoir 140 comprises a thermoplastic polyurethane (TPU) or low-density polyethylene (LDPE). In this embodiment, the reservoir adapter 133 comprises a relatively rigid material compared to the sleeve 130, fluid reservoir 140 and catheter 120, for example high-density polyethylene (HDPE), and thus provides a handling element to facilitate movement of the proximal end 131 of the sleeve 130 up and down the catheter 120.

The pouch 170 is formed from a front wall (not shown-cut away) and rear wall 171 of identical shape and size, and a peripheral seal joining the periphery of the walls to form the pouch 170 and containing its contents (i.e. the closed catheter assembly). The pouch 170 thereby provides a sterile environment for the catheter.

The peripheral seal defines a base 172a, a left lateral edge 172b, a right lateral edge 172c and an upper edge 172d of the pouch 170. The left lateral edge 172b and right lateral edge 172c being defined as the left and right sides of the pouch 170 when viewing the pouch 170 with the rear wall 171 behind the front wall, the base 172a at the bottom of the pouch 170 and the upper edge 172d at the top of the pouch 170. The peripheral seal thus defines a pouch 170 that is generally rectangular and suitably has a width between the lateral edges of between 60-110 mm, preferably 90-110 mm, for example about 100 mm, and a height from the base 172a to the upper edge 172d of between 100 to 250 mm, preferably 120 to 140 mm, for example about 130 mm. The base 172a defines the bottom of the pouch 170 in use, and the upper edge 172d the top. However, in other embodiments alternative shapes and sizes could be conceived, such as elliptical or circular walls forming correspondingly shaped pouches, or even a tubular pouch or pouch with more than two walls, e.g. a prism or other polygon.

The peripheral seal may be formed from any suitable bonding/sealing methods such as chemical adhesives, chemical bonding, ultrasonic bonding, or mechanical bonding. In alternative embodiments, the pouch 170 may be formed from a single piece of material. The pouch 170 may then be formed by folding this single piece of material along a pouch fold line to form the walls. In addition, in some embodiments any number of folds may be used to form the pouch 170. For example, none of the edges may comprise a fold, one edge may comprise a fold (as in the illustrated example), or two or more of the edges may comprise a fold.

In this embodiment, the pouch 170 is formed from an opaque material and as such provides a discrete package for its contents which can make the user more comfortable in carrying the packaged catheter assembly 100 in day-to-day life. In addition, the exterior of the pouch 170 may have a textured or printed appearance, for example a matte appearance, and feel to reduce the impression that it is a medical device. The front and/or rear wall of the pouch may comprise a wetting marker (not shown) which provides a visual/tactile guide to help the user locate the fluid reservoir 140 within the closed pouch and activate it to release wetting fluid.

In this embodiment, the pouch 170 also comprises an interaction region. The interaction region forms the top part of the pouch 170 and spans between the right lateral edge 172c and left lateral edge 172b. The interaction region is used to provide access to the pouch through/near to the upper edge 172d. In this embodiment, the interaction region comprises a tapered tear-away region 175. The tear-away region 175 comprises a tear start 176 at the point the left lateral edge 172b meets the tear-away region 175, a tear stop 177 at a corresponding point on the right lateral edge 172c, and a tear line 178 spanning between the tear start 176 and tear stop 177. The tear line 178 comprises a line of weakness in the walls of the pouch 170, which may be formed by any suitable means but in this embodiment is laser etched. The tear start 176 comprises a notch to provide an area of weakness in the peripheral seal. This allows the tear-away region 175 to be separated from the pouch 170 by tearing the walls apart from the tear start 176 to the tear stop 41 along the tear line 178. Thus, the tear is provided beneath the location of the upper edge 172d and thus the pouch 170 may be opened provided access to its contents. In other embodiments, the tear stop 177 may comprise a small aperture in the walls to prevent further tearing of the pouch 170.

In order to provide the tapered shape, the tear-away region 175 extends upwards on the left side approximately 50% more than it does on the right side. The left side of the tear-away region 175 comprises a circular aperture 179 sized to allow a finger to pass through and grip the tear-away region 175. To ensure the pouch 170 is fully sealed, the front wall and rear wall 171 are sealed to one another over the entire tear-away region 175. In other embodiments, other seals such as a zip-lock arrangement, or hook and hook, hook and loop (VELCRO®) or the like may be used to provide access to the pouch (or to reseal the pouch after opening), and of course, the sizes and shapes set out above are exemplary.

In this embodiment, the catheter 120 is arranged within the pouch 170 in a curved and S-shaped configuration with two opposing turns of at least 180 degrees along the length of the catheter 120. The distal end 122 of the catheter 120 is arranged adjacent to the inner left lateral edge 172b near the upper edge 172d of the pouch 170. The catheter 120 extends down the inside of the pouch 170 into a first turn adjacent the base 172a of the pouch and then extends back up the pouch 170 towards the distal end 122 of the catheter 120. The catheter 120 then comprises a second turn adjacent the fluid collection bag 110 (as described below) and at a height in the pouch 170 corresponding to the position of the distal end 122 of the catheter 120. Finally, the catheter 120 extends down the inner right lateral edge 172c of the pouch 170 with the proximal end 121 of the catheter 120 resting approximately half the way down the height of the pouch 170.

In this embodiment, each of the first and second turns in the catheter 120 comprise turns of at least 180 degrees, and preferably 180-220 degrees, for example 200 degrees. Each of the first and second turns has a radius of curvature approximately equal to one third the width of the pouch and as such, each turn spans two-thirds the width of the pouch. Thus, the S-shape of the catheter 120 helps to maintain the contents of the pouch 170 in a stable position before use and also protects the catheter 120 from damage, for example kinking, due to mechanical stresses imposed on the pouch 170. Of course, the catheter 120 straightens as it is pulled out of the pouch, as described below.

In this embodiment, the fluid collection bag 110 is provided in a stowed configuration, which in this embodiment is a folded configuration, within the pouch 170 and is retained in the folded configuration by the walls of the pouch 170 which provide compressive pressure to the folded fluid collection bag 110. In other embodiments, the fluid collection bag 110 may be retained in the folded configuration by any other suitable means, such as an elastic band, paper band, or closure. The folded fluid collection bag 110 is generally cylindrical in shape and is arranged within the pouch 170 just below the tear line 178 and with an axis of the cylindrical folded fluid collection bag 110 approximately parallel to the tear line 178. The inlet 150 of the bag 110 receives the distal end 122 of the catheter 120 adjacent to the left lateral edge 172b of the pouch 170, and the opposite end of the bag 110 is positioned adjacent to the right lateral edge 172c of the pouch 170. The fluid collection bag 110 is therefore arranged adjacent to the opening formed in the pouch 170 by the tear-away region 175 as described further below and spans between the left lateral edge 172b and right lateral edge 172c.

In this embodiment, the sleeve 130 is arranged around the catheter 120 and covering the majority of the length of the catheter 120. The sleeve 130 has a diameter of 2 to 4 times the diameter of the catheter 120. The distal end 132 of the sleeve 130 is attached to the inlet 150 of the fluid collection bag 110, and as described above, the proximal end 131 of the sleeve 130 is attached to the reservoir adapter 133. The reservoir adapter 133 is positioned at the proximal end 121 of the catheter 120 and adjacent the right lateral edge 172c of the pouch 170. The fluid reservoir 140 is engaged by the reservoir adapter 133 and extends from the proximal end 121 of the catheter 120 down the inner right lateral edge 172c of the pouch 170 to the base 172a of the pouch 170. The fluid reservoir of this embodiment is attached to a wall of the pouch, e.g. by adhesive, to prevent it being accidentally removed from the pouch. The fluid reservoir 140 can therefore be activated by pressing the walls of the sealed closed pouch 170 together adjacent the base 172a and right lateral edge 172c. Of course, in other embodiments, the catheter 120, fluid collection bag, sleeve 130 with reservoir adapter 133 and fluid reservoir 140 may be arranged differently within the pouch 170.

Referring to FIG. 4, in this embodiment, the fluid collection bag 110 comprises a front panel 111, a rear panel of identical shape and size (not shown), and a peripheral bond joining the periphery of the panels to form the bag 110. The front panel 111 and rear panel are both transparent or translucent in this embodiment. The peripheral bond defines a base 112, a right lateral edge 113, a left lateral edge 114 and an upper edge 115. The right lateral edge 113 and left lateral edge 114 being defined as the right and left sides of the bag 110 when viewing the bag 110 with the rear panel behind the front panel 111, the base 112 at the bottom of the bag 110 and the upper edge 115 at the top of the bag 110. The peripheral bond thus defines a bag 110 that, when unfolded, is generally rectangular and suitably has a width between the lateral edges of at least 10, 12, 15 or 20 cm. The bag may have a width no more than 25, 20, or 15 cm. Preferably, the width is between 10 and 20 cm, most preferably between 12 and 15 cm. A height from the base 112 to the upper edge 115 may be at least 25, 30, or 40 cm. The height of the bag may be no more than 45, 40, 35 or 30 cm. The height may be between 25 and 40 cm, 30 and 35 cm, or most preferably 33 and 35 cm. The bag may be configured to hold at least 500, 700, or 1000 ml of fluid, and may hold no more than 1000 ml. For example, it may hold at least 700 ml of fluid. The base 112 defines the bottom of the bag 110 in use, and the upper edge 115 the top. However, in other embodiments alternative shapes and sizes could be conceived, such as elliptical or circular panels forming correspondingly shaped bags, or even a tubular bag or bag with more than two panels, e.g. a prism or other polygon.

In this embodiment, the panels comprise a flexible plastics material, for example polypropylene (PP), polyethylene terephthalate (PET), low density polyethylene (LDPE), metalized polyester (MET PET), orientated polypropylene (OPP) or polyvinyl chloride (PVC).

In this embodiment, the inlet 150 is positioned at the corner of the peripheral bond where the upper edge 115 and left lateral edge 114 meet. A neck 151 is formed adjacent to the inlet 150 in the bag 110 by the peripheral bond which comprises two triangular projections 117a, 117b into the bag 110, a left edge projection 117a along the left lateral edge 114 and an upper edge projection 117b along the upper edge 115.

In this embodiment, the left edge projection 117a is a right-angle triangle with its right-angled vertex projecting into the bag 110 and the other vertices along the left lateral edge 114. The left edge projection 117a is approximately one quarter the length of the left lateral edge 114 and begins approximately 10% along the length of the left lateral edge 114 from the upper edge 115. The left edge projection 117a extends from this point approximately 10% across the width of the bag 110 angled slightly below perpendicular to the left lateral edge 114 before turning 90 degrees at the right-angled vertex and returning to meet the left lateral edge 114.

In this embodiment, the left edge projection 117a also comprises a rib 117c spanning from its right-angled vertex to a point on the left lateral edge 114 and approximately midway along the length of the left edge projection 117a. The rib 117c comprises a region which preferentially folds to help with maintaining the shape of the bag 110 both when folded and unfolded and also makes it easier for a user to grasp/grip the bag 110.

In this embodiment, the upper edge projection 117b is a right-angle triangle. The right-angled vertex projecting into the bag 110 and the other vertices along the upper edge 115. The upper edge projection 117b is approximately 90% the length of the upper edge 115 and begins approximately 10% along the length of the upper edge 115 from the left lateral edge 114 and ends at the right lateral edge 113. The upper edge projection 117b extends from where it meets the upper edge 115 approximately 10% down the length of the bag 110 angled slightly right of perpendicular to the upper edge 115 before turning through 90 degrees towards the right lateral edge 113 at the right-angled vertex and then returning to meet the right lateral edge 113.

Thus, as described above, in this embodiment the right-angled vertices of the left edge projection 117a and upper edge projection 117b define the narrowest point of the neck 151.

In this embodiment, the peripheral bond further comprises two finger holes 116 to allow the bag 110 to be easily handled by a user. One finger hold is positioned in the upper edge projection 117b adjacent to its right-angled vertex. The other finger hole 116 is at the corner of the bag 110 where the right lateral edge 113 meets the base 112, and the peripheral bond truncates the corner of the bag 110 to accommodate this finger hole 116. In other embodiments, the finger holes 116 may comprise a series of ridges, or otherwise easily gripped surface or feature to allow the user to easily handle the bag 110. In addition, there may be different numbers of finger holes 116, such as only one or more than two, and they may be placed in different locations on the bag 110.

In this embodiment, the fluid collection bag 110 further comprises a fill level indicator 118 comprising fill markers which allow the user to measure the volume of fluid contained within the fluid collection bag 110. Due to the shape of the bag 110, the cross-sectional area of the bag 110 may vary along the length of the bag 110 from the base 112 to upper edge 115. As such, the fill level indicator may comprise irregularly spaced fill markers to account for this. The fill markers may indicate, either the proportion of the volume of the bag which is filled with fluid, for example 10%, 25%, 50%, 75%, 100% of a safe fill level, or the absolute volume of fluid, for example 100 ml, 200 ml, 300 ml, etc. as required or as is convenient for the user.

Referring to FIGS. 1 and 2, in this embodiment, the packaged catheter assembly 100 is provided with the folded fluid collection bag 110, catheter 120, sleeve 130 and fluid reservoir 140 all contained with the pouch 170 as mentioned above. To wet the catheter 120 so that it is ready for use, the user may release the wetting fluid from the fluid reservoir 140 into the sleeve 130. This may be done by any suitable means, but preferably by applying pressure to the outside of the pouch 170 at a region corresponding to the position of the fluid reservoir 140. Alternatively, if the fluid reservoir is not attached to the pouch, the user may first remove the fluid collection bag 110, catheter 120, sleeve 130 and fluid reservoir 140 from the pouch 170 (as described below) and then activate the fluid reservoir 140 by pressing the activation marker 144.

As described above, in this embodiment, the fluid reservoir 140 is configured to rupture under external pressure and create a tear 141 in the fluid reservoir 140 at a position that is enclosed by the reservoir adapter 133. As such, the wetting fluid is released into the reservoir adapter 133 where it is able to flow through the bore of the reservoir adapter 133 along the length of the catheter 120 between the catheter 120 and sleeve 130.

Referring to FIGS. 3 and 4, in this embodiment, the user may then access the catheter 120 by creating an opening in the pouch 170 using the interaction region. The aperture 179 is grasped in one hand, and the pouch 170 below the tear line 178 in another, and the tear-away region 175 is torn from the tear start 176 to the tear stop 177 along the tear line 178. Thus, the pouch 170 is torn between the left lateral edge 172b and right lateral edge 172c at a location below the upper edge 172d, as such, an opening is formed in the pouch 170 that may be used to access its contents. In this embodiment, the tear-away region 175 is fully separated from the pouch 170 to improve access to the contents of the pouch 170. In other embodiments, the tear-away region 175 may not be fully separated from the pouch 170 to reduce the number of separate parts and make the pouch 170 easier to handle. Other embodiments may also feature other ways to access the pouch for example a zip-lock seal.

In this embodiment, the closed catheter assembly (i.e. catheter 120 attached to the sleeve 130, reservoir adapter 133, fluid reservoir 140, and fluid collection bag 110) may then be removed from the pouch 170. The folded fluid collection bag 110 is easily accessible to the user as it is directly accessible via the opening in the pouch 170. To withdraw the closed catheter assembly from the pouch 170 the user inverts the pouch, so that the fluid collection bag 110 is towards the bottom of the pouch and pulls it downwards out of the pouch 170, at which point the fluid will flow down through the sleeve.

In this embodiment, the fluid collection bag 110 may then be unfolded so that it is ready to accept fluid from the catheter 120. The catheter 120 may be handled via the sleeve 130 and/or the reservoir adapter 133.

In this embodiment, the fluid reservoir 140 must be removed from the reservoir adapter 133 to allow the proximal end of 121 of the catheter 120 to be withdrawn from the sleeve 130 and inserted into the body. The fluid reservoir 140 is removed from the reservoir adapter 133 by simply pulling the reservoir adapter 133 out of the pouch and hence away from the fluid reservoir 140 which is retained within the pouch. Now that the fluid reservoir 140 is removed from the reservoir adapter 133, the catheter 120 is able to be passed through the reservoir adapter 133 for use. In other embodiments, the fluid reservoir 140 may be actively disengaged by twisting or otherwise activating the reservoir adapter 133 to release the sleeve 130 for example.

The steps of releasing wetting fluid into the sleeve 130, unfolding the fluid collection bag 110, and disengaging the fluid reservoir 140 as described above may be performed in any order depending on the construction of the packaged catheter assembly and the preference of the user.

Referring to FIG. 4, in this embodiment, the catheter 120 is now ready for use. The sleeve 130 may be used to handle the catheter 120 and the reservoir adapter 133 may be used to progressively introduce the proximal end 121 of the catheter 120 into the urethra until fluid flows through the catheter 120 from the bladder. Fluid flowing through the catheter 120 is collected in the fluid collection bag 110 and the volume of fluid contained in the bag can be monitored using the fill level indicator. After use, in this embodiment, as the catheter 120 is withdrawn from the body, the reservoir adapter 133 may be used to cover the length of the catheter 120 with the sleeve 130 and the packaged catheter assembly 100 may then be disposed of.

As mentioned above, in this embodiment the catheter 120 and sleeve 130 are in fluid communication with the fluid collection bag 110 via the inlet 150. As described below, fluid can pass from the distal end 132 of the sleeve 130 into the fluid collection bag 110 via the inlet 150 (optionally via the catheter 120, see FIG. 7) and also from the distal end 122 of the catheter 120 into the fluid collection bag 110 via the inlet 150 (optionally via the sleeve 130 see FIG. 6). The configuration of the catheter 120, sleeve 130, inlet 150 and bag 110 is relatively flexible and many different configurations could be employed in embodiments of the invention.

Referring to FIG. 5, in a first example of the configuration for the catheter 120, sleeve 130 and inlet 150 fluid passes directly from the sleeve 130 to the inlet 150 and directly from the catheter 120 to the inlet 150. In this example, a right lateral side 123 of the catheter 120 is attached to a right side 155 of the inlet 150 and there is a gap between a left lateral side 124 of the catheter 120 and the respective left side 156 of the inlet 150. In addition, the distal end 122 of the catheter 120 is within the inlet 150, and the sleeve 130 is sealed around a periphery of the inlet 150. As such, fluid travelling within the sleeve 2 is directed into the inlet 150 directly via the gap between the left lateral side 124 of the catheter 120 and the left side 156 of the inlet 150. In addition, fluid travelling within the catheter 3 passes out the distal end 122 and into the inlet 150 directly. In other examples, the catheter 120 may be positioned differently and may be attached to the left side 156 of the inlet 150, or to a front or rear side.

Referring to FIG. 6, in a second example fluid passes from the catheter 120 into the sleeve 130 and then into the inlet 150. In this example, the right lateral side 123 of the catheter 120 is attached to a left side 134 of the sleeve 130 and a distal end 122 of the catheter 120 is not within the inlet 150. As in the first example, the sleeve 130 is sealed around a periphery of the inlet 150. As such, fluid travelling within the sleeve 2 is directed into the inlet 150 directly and fluid travelling within the catheter 3 passes out the distal end 122 into the sleeve 130 and then from there into the inlet 150. In other examples, the catheter 120 may be positioned differently and may be attached to a left side 135 of the sleeve 130, or indeed to a front or rear side. Furthermore, the distal end 122 of the catheter 120 may be blocked and an outlet (not shown) may be provided in a lateral side of the catheter 120 to allow fluid to pass out into the sleeve 130.

Referring to FIG. 7, in a third example fluid passes from the sleeve 130 into the catheter 120 and then into the inlet 150. The configuration of this example is similar to the first example shown in FIG. 12 and so only the differences are described. In this example, the inlet 150 is sealed between the left side 156 and right side 155 such that the catheter 120 is sealed on all sides. As such, fluid may only pass from the sleeve 130/catheter 120 into the inlet 150 if it is within the catheter 120. The left lateral side 124 of the catheter 120 comprises an oval sleeve intake opening 125 adjacent to the inlet 150 which permits the passage of fluid travelling within the sleeve 3 into the catheter to join fluid travelling within the catheter 2. From here, the fluid 2, 3 passes out the distal end 122 of the catheter 120 and into the inlet 150. Many other configurations may be possible to achieve the same effect, for example, the sleeve 130 may be attached to the outer sides of the catheter 120 itself.

Referring to FIG. 8, a second embodiment of a packaged catheter assembly shares many of the features of the first embodiment described above, in particular, the pouch 170, fluid collection bag 110, catheter 220, sleeve 230 and reservoir adapter 233 are identical and so only differences in the features are described below, and like numerals are used to denote like features.

In this embodiment, the fluid reservoir 240 further comprises a reservoir jacket 242 configured to enclose the fluid reservoir 240. The reservoir jacket 242 comprises an impermeable material, for example, a thermoplastic polyurethane (TPU) or low-density polyethylene (LDPE). The reservoir jacket 242 further comprises a wetting aperture 243 arranged at a position corresponding to the first end of the fluid reservoir 240, that is, the end engaged by the reservoir adapter 233 and closest to the sleeve 230 and catheter 220. The reservoir jacket 242 is therefore configured to direct wetting fluid released from the fluid reservoir 240 into the sleeve 230 and onto the catheter 220 via the wetting aperture 243. This helps ensure that wetting fluid released by the fluid reservoir 240 is directed into the sleeve 230, for example in the situation where the fluid reservoir 240 inadvertently ruptures at a position that is not enclosed by the reservoir adapter 233.

Furthermore, as the reservoir jacket 242 comprises the wetting aperture 243, pressure applied to the fluid reservoir 240 to rupture it does not also rupture the reservoir jacket 242, as any increase in pressure within the reservoir jacket 242 is relieved through the wetting aperture 243. Thus, the reservoir jacket 242 remains impermeable to wetting fluid and can direct it into the sleeve 130 irrespective of the location in which the fluid reservoir 140 ruptures.

In this embodiment, the reservoir jacket 242 is transparent or translucent and allows the activation marker 244 to be visible through the reservoir jacket 242. In other embodiments, the reservoir jacket 242 may be opaque or may otherwise obscure the activation marker 244. In such embodiments, an activation marker may be placed on the reservoir jacket 244 itself.

Referring to FIGS. 9-22, a third embodiment of a catheter assembly 300 is shown. Again, like features are given like reference numerals.

The assembly 300 comprises a fluid collection bag 310, a catheter 320, a sleeve 330, and a fluid reservoir 340. The catheter 320 has a proximal end 321 for insertion into the body and a distal end 322, and the fluid collection bag 310 is arranged to receive fluid from the distal end 322 of the catheter 320. The sleeve 330 encloses the catheter 320 along the length of the catheter 320 from the proximal end 321 to the distal end 322. The sleeve 330 is also in fluid communication with the fluid collection bag 310. The fluid reservoir 340 is arranged at the proximal end 321 of the catheter 320 and is configured to release wetting fluid into the sleeve 330 to wet the catheter 320.

In this embodiment, the catheter 320 is a male urinary catheter made from a hydrophilic thermoplastic elastomer (TPE). The sleeve 330 of this example is a low-density polyethylene (LDPE). Obviously those skilled in the art will be able to select suitable alternative examples.

In this embodiment, the wetting fluid is water and interacts with the hydrophilic surface of the catheter 320 to render it lubricious. In other embodiments, other wetting fluids may be used and they may be polar (e.g. water-based) or non-polar (e.g. oil-based) depending on the catheter's surface properties.

In this embodiment, the fluid collection bag 310 comprises a front panel 311, a rear panel of identical shape and size (not shown), and a peripheral bond joining the periphery of the panels to form the bag 310. The peripheral bond defines a base 312, a right lateral edge 313, a left lateral edge 314 and an upper edge 315. The right lateral edge 313 and left lateral edge 314 being defined as the right and left sides of the bag 310 when viewing the bag 310 with the rear panel behind the front panel 311, the base 312 at the bottom of the bag 310 and the upper edge 315 at the top of the bag 310. The peripheral bond thus defines a bag 310 that is generally rectangular and suitably has a width between the lateral edges of between 10 and 20 cm, and most preferably between 12 and 15 cm, and a height from the base 312 to the upper edge 315 of between 25 and 40 cm, 30 and 35 cm, or most preferably 33 and 35 cm. The bag is thus configured to hold at least 700 ml of fluid released from the user through the catheter safely. The base 312 defines the bottom of the bag 310 in use, and the upper edge 315 the top. However, in other embodiments alternative shapes and sizes could be conceived, such as elliptical or circular panels forming correspondingly shaped bags, or even a tubular bag or bag with more than two panels, e.g. a prism or other polygon.

In this embodiment, the panels comprise a flexible plastics material, for example polypropylene (PP), polyethylene terephthalate (PET), low density polyethylene (LDPE), metalized polyester (MET PET) orientated polypropylene (OPP), or polyvinyl chloride (PVC).

In this embodiment, the corners of the peripheral bond at either end of the left lateral edge 314 each comprise finger holes 316 to allow the bag 310 to be easily handled by a user. Where the left lateral edge 314 meets the base 312, the peripheral bond truncates the corner of the bag 310 to accommodate the finger hole 316, and where the left lateral edge 314 meets the upper edge 315, the peripheral bond forms a square projecting into the interior of the bag 310 with the finger hole 316 at its centre. In other embodiments, the finger holes 316 may comprise a series of ridges, or otherwise easily gripped surface or feature to allow the user to easily handle the bag 310. In addition, there may be different numbers of finger holes 316, such as only one or more than two, and they may be placed in different locations on the bag 310.

In this embodiment, the upper edge 315 of peripheral bond comprises a neck 350 which spans the middle 50% of the length of the upper edge 315. In other embodiments, it may be located a different point on the upper edge 315 or peripheral bond, such as on the left or right lateral edges 314, 313, or base 312, and may have a different size in relation to the rest of the bag 300 or edge in which it is situated. The neck 350 projects out from the plane of the bag 310. The neck 350 is triangular and comprises a left sloped edge 351 and a right sloped edge 352 which project from the upper edge 315 and converge at an inlet 353. The inlet 353 is connected to the catheter 320 and sleeve 330 and is configured to received fluid from the catheter 320 and sleeve 330 as described below.

In this embodiment, the upper edge 315 also comprises an outlet 360 arranged between the neck 350 and the right lateral edge 313. The outlet 360 comprises a rectangular spout 361 that projects from the upper edge 315 the same distance as the inlet 353. The outlet 360 also comprises a pull-ring 362 attached to the spout 361 and disposed between the spout 361 and neck 350. The pull-ring 362 has the shape of a right-angled triangle, with two perpendicular sides and a hypotenuse 363. The hypotenuse 363 is parallel and adjacent to the right sloped edge 352 of the neck 350. In other embodiments, the pull-ring 362 may be any suitable shape or size-such as circular, rectangular or square. This helps to protect the neck 350, inlet 353 and outlet 360 as they project out from the fluid collection bag 310 together without significant gaps between them.

The outlet 360 also comprises a point of weakness, which in this embodiment is a notch 364 in the peripheral bond at the point where the spout 361 meets the right lateral edge 313. This helps ensure that the opening 316 is formed cleanly and does not propagate down the right lateral edge 313 of the pouch. In other embodiments, the outlet may comprise a tear line which comprises a weakened region of the outlet 316. The notch 364 may be positioned at an end of the tear line and as such, the tear line Further assists with forming the opening 316. The tear line may be formed by any suitable means, such as laser scoring.

In this embodiment, the distal end 322 of the catheter 320 and sleeve 330 are in fluid communication with the bag 310 via the inlet 353, as described in more detail with reference to FIGS. 20-22 below. At the other end of the sleeve 330, corresponding to the proximal end 321 of the catheter 320, the sleeve 330 comprises a reservoir adapter in the form of a handling element 331. In this embodiment, the handling element 331 is cylindrical and has a radius at least twice that of the catheter 320 and a length no longer than the radius of the handling element 331. In other embodiments, the handling element 331 may have different dimensions or different shapes entirely.

In this embodiment, the sleeve 330 further comprises a tube 332 extending from the handling element 331 and defining an aperture 333 which permits the catheter 320 to exit the sleeve 330 as described below. The tube 332 has a radius greater than that of the catheter but less than that of the handling element 331, as such, it protects the catheter 320 as it emerges from the aperture 333 and reduces the likelihood that it is inadvertently touched by the user's hand which is grasping the handling element 331.

In this embodiment, the aperture 333 releasably engages with the fluid reservoir 340 through a screw fitting. The fluid reservoir 340 is therefore received by the aperture 333. Further, the fluid reservoir 340 is moveable axially with respect to the thread of the aperture 333 between positions where it engages the thread and does not engage the thread respectively. This ensures that the fluid reservoir 340 is less likely to be unintentionally removed from the sleeve 330 as it requires two different movements to be removed. In other embodiments, different types of engagement could be used such as bayonet or push-fit fittings.

In this embodiment, the fluid reservoir 340 is an elongate cylinder filled with a wetting fluid. The fluid reservoir also comprises two wings 341 which project from opposite sides of the cylindrical fluid reservoir 340 in a plane parallel to the axis of the fluid reservoir. The wings 341 of this particular embodiment are rectangular with rounded corners and extend along most of the length of the reservoir 340 with a width approximately equal to the diameter of the reservoir. The wings 341 provide gripping surfaces which allow a user to easily grasp and manipulate the fluid reservoir 340. In other embodiments, alternative gripping surfaces may be provided and/or the wings 341 may comprise other features that allow the fluid reservoir 340 to be handled easily, such as pull-rings or finger holes.

In this embodiment, the fluid reservoir 340 comprises a flexible plastics material, for example a hard moulded thermoplastic such as HDPE.

In use, the fluid reservoir 340 retains the wetting fluid within it until the fluid reservoir 340 is burst which then allows wetting fluid to enter the sleeve the sleeve 330 adjacent to the proximal end 321 of the catheter 320, this ensures that the proximal end 321 is well wetted which improves comfort and reduces risk of injury when using the catheter 320 as the proximal end 321 is the first part to be introduced into the body. In this embodiment, the fluid reservoir 340 is burst by pressing the fluid reservoir 340 into the handling element 331 and thus towards the proximal end 321 of the catheter 320. This causes the fluid reservoir 340 to be pierced by a spike (not shown) on the end of the sleeve 330/handling element 331. As mentioned above, the fluid reservoir 340 is movable axially with respect to the aperture 333, in this embodiment, the spike is configured to pierce the fluid reservoir 340 when it is in a position that the thread of the aperture 333 engages the thread of the fluid reservoir 340 allowing it to be unscrewed and removed. Thus, the fluid reservoir 340 may not be removed from the sleeve 330 without it first being pierced and releasing wetting fluid into the sleeve 330 to wet the catheter 320 for use.

Referring to FIGS. 10-12, the efficiency of the wetting fluid in wetting the catheter 320 varies depending on the orientation of the catheter 320 and sleeve 330 as well as how the user is gripping or holding the assembly 300. As such, to increase the likelihood that the catheter 320 is adequately wetted, in this embodiment the fluid reservoir 340 is configured to release excess wetting fluid into the sleeve 330 ensuring there is more than adequate wetting fluid to wet the catheter 320 irrespective of how the assembly is being held or its orientation. If this excess wetting fluid is not required for wetting the catheter 320, it may then flow through the sleeve 330 and into the fluid collection bag 310 via the inlet 353 to form a pool 3 in the bag 310. Advantageously, this ensures the excess wetting fluid does not get out of the assembly 300 and onto the user or surfaces of the assembly 300 that the user may need to hold/manipulate. This improves the ease of use of the assembly 300 and makes it more convenient to carry while ensuring the catheter 320 is adequately wetted.

Referring to FIG. 11, after the wetting fluid has been released from the fluid reservoir 340 into the sleeve 330, in this embodiment the fluid reservoir 340 may be removed from the assembly 300. In this embodiment, the fluid reservoir 340 is simply pulled off the sleeve 330 by separating the handling element 331 from the fluid reservoir 340. In other embodiments, a different action may be required such as releasing a locking element or twisting/unscrewing the fluid reservoir 340.

Referring to FIGS. 11 and 12, in this embodiment after removal of the fluid reservoir 340, the aperture 333 is exposed which allows the proximal end 321 of the catheter 320 to pass out of the sleeve 330. The handling element 331 can then be used to guide the proximal end 321 into the body without the user having to touch the catheter 320, this reduces the risk of infection to the user. The user can then progressively pass more of the catheter 320 out of the sleeve 330 and into the body by bunching the sleeve 330. Once the catheter 320 has been sufficiently inserted into the body, fluid may pass through the catheter 320 out of the body, and into the fluid collection bag 310 via the inlet 353 as described in greater detail below with reference to FIGS. 20-22.

Referring to FIGS. 13-15, after use of the catheter 320, the user may wish to empty the contents of the fluid collection bag 310. This can be desirable in order to reduce the size and weight of the used bag 310 and/or to dispose of the bag 310. To allow the bag 310 to be emptied, in this embodiment the outlet 360 may be removed from the bag 310 to form an opening 316 in the peripheral bond between the neck 350 and notch 364 as described below.

To form the opening 316, in this embodiment the user first folds the neck 350 onto the front panel 311 of the bag 310 creating a neck fold-line 354 parallel to the base 312 of the peripheral bond and spanning between the end of the left sloped edge 351 and the end of the right sloped edge 352 distal the inlet 353. Advantageously, this helps to ensure fluid in the fluid collection bag 310 is less likely to pass back into the sleeve 330 via the inlet 353 and also ensures the opening 316 is correctly formed while the outlet 360 is being removed from the fluid collection bag 310. In other embodiments, the same effect may be achieved by different means such as deforming, rolling or folding the neck 350 in different ways.

In this embodiment, once the neck 350 is folded, the outlet 360 is exposed and projects beyond the edge of the bag 310 which is now defined by the neck fold-line 354. The pull-ring 362 may then be grasped by the user and pulled in a direction parallel to the neck fold-line 354 and away from the left lateral edge 314 to separate the outlet 360 from the fluid collection bag 310 forming the opening 316. Advantageously, the peripheral bond preferentially tears from a point between the right sloped edge 352 of the neck 350 and outlet 360 due to the neck fold-line 354 which reinforces the peripheral bond around the neck 350 and reduces tearing forces on the right sloped edge 352. In addition, the shape of the pull-ring 362 means that when moved away from the inlet 353, shearing force is concentrated at the point the outlet 360 meets the upper edge 315. This ensures the opening 316 is created in a controlled manner.

In this embodiment, the notch 364 provides a second point on the peripheral bond which preferentially forms one end of the opening 316. Thus, the outlet 360 may be easily, conveniently, and reliably removed from the fluid collection bag 310 to form a clean opening 316 in the peripheral bond. This allows the user to easily empty the contents of the bag 310 and reduces the risk of spilling the contents of the bag 310 during emptying.

Referring to FIGS. 16-19, in this embodiment, the catheter assembly also comprises a pouch 370 which contains the fluid collection bag 310, catheter 320, sleeve 330 and fluid reservoir 340 within it. The pouch 370 is formed from a single rectangular piece of material. The pouch is formed by folding this single piece of material along a pouch fold-line 373a spanning its width at a point midway along its length to form a front wall 371 and a rear wall 372 of the pouch 370. A peripheral seal 373 exists between the front wall 371 and rear wall 372 around the edges of the front wall 371 and rear wall 372 (except the edge corresponding to the pouch fold-line 373a) to form the pouch 370 and contain the contents of the pouch 370. In this embodiment, the peripheral seal 373 comprises all edges of the pouch other than the pouch fold-line 373a and is formed through heat sealing the front wall 371 to the second wall 372, but in other embodiments any suitable bonding may be used such as Velcro®, chemical adhesives, chemical bonding, ultrasonic bonding, or mechanical bonding. In addition, in some embodiments any number of folds may be used to form the pouch 370. For example, none of the edges may comprise a fold and instead one edge may be formed by a peripheral join which is not configured to be separated during opening of the pouch—such as through bonding the walls in a similar manner to the peripheral seal but in a way that is stronger than the peripheral seal. Alternatively, one edge may comprise a fold (as in the illustrated example), or two or more of the edges may comprise a fold.

In this embodiment, the pouch 370 is formed from an opaque material and as such provides a discrete package for the catheter assembly 300 which can make the user more comfortable in carrying the catheter assembly 300 in day-to-day life. In addition, the exterior of the pouch 370 may have a textured or printed appearance and feel, such as a matte finish to reduce the impression that it is a medical device.

In this embodiment, the pouch 370 also comprises an absorbent layer 374. The absorbent layer 374 is a layer of absorbent material that lines the inside surfaces of both the front wall 371 and rear wall 372. As such, the absorbent layer 374 is a single sheet of absorbent material folded at the pouch fold-line 373a. In other embodiments, the absorbent layer 374 may only line one of the front wall 371 or rear wall 372, or may only be present at certain locations in the pouch 370. In other embodiments, the absorbent layer 374 may be replaced by an absorbent that is not shaped like a layer, such as an absorbent sachet or absorbent strip of material.

In this embodiment, the front wall 371 comprises a front opening tab 375 and the rear wall 372 comprises a rear opening tab 376. Both the front opening tab 375 and rear opening tab 376 project from an edge of the pouch 370 opposite the pouch fold-line 373a. Each of the tabs 375, 376 of this particular embodiment are trapezoid in shape with two sides 378, a base 379 and a top 380, and a height between the base 379 and top 380, wherein the length of the base 379 is greater than the length of the top 380 and also at least twice as great as the height. The tabs 375, 376 are arranged with their respective bases 379 parallel to the pouch fold-line 373a and the base 379 of each tab 375, 376 forms an edge of the pouch 370.

In this embodiment, the tabs 375, 376 are positioned at corresponding positions on the edge of the pouch 370 and therefore overlap one another and without any offset or overhang. This helps to reduce the form factor of the pouch 370. In other embodiments, the tabs 375, 376 may be positioned at different positions to introduce an offset or overhang between the tabs 375, 376. This may be done to make it easier to grasp one of the tabs 375, 376 without the other.

In this embodiment, each tab 375, 376 is also configured to be folded about its base against either the front wall 371 or rear wall 372. Both tabs 375, 376 may be folded against the front wall 371, or both against the rear wall 372. This allows the user to reduce the form factor of the pouch 370. Furthermore, where both tabs 375, 376 are folded against the same wall, the folding of the tabs 375, 376 in the same direction provides resistance to the inadvertent separation of the tabs 375, 376 and resultant opening of the pouch 370.

Each tab 375, 376 also comprises a gripping hole 377. Each gripping hole 377 of this particular example is in the shape of a stadium, that is a rectangle with semicircles replacing two opposite sides. The parallel sides of each gripping hole 377 are parallel to the base and top of the tabs 375, 376 and form apertures which allow the user to easily grasp each tab 375, 376. Of course, alternative shapes of gripping hole can be readily imagined.

In use, the pouch 370 is initially provided sealed with the fluid collection bag 310, catheter 320, sleeve 330 and fluid reservoir 340 within it. To access the contents, the user grasps each of the tabs 375, 376 using the gripping holes 377 and pulls the tabs 375, 376 away from one another. This in turn pulls the front wall 371 away from the rear wall 372 and breaks a frangible portion of the peripheral seal 373 which comprises the edges of the pouch 370 that do not correspond to the pouch fold-line 373a.

Referring to FIG. 18, as the pouch 370 is folded about the pouch fold-line 373a, the front wall 371 and rear wall 372 preferentially open out to a flat position. In this embodiment, a flat position is defined as when the angle between the front wall 371 and rear wall 372 is less than 1 degree. This opening of the pouch 370 exposes the absorbent layer 374 as well as the contents of the pouch 370 for use as described above in relation to the first embodiment. Advantageously, any leakage from the fluid reservoir 340 may be absorbed by the absorbent layer 374 preventing accidental wetting of items/surfaces which are to be handled by the user.

During preparation and use of the catheter 320, the opened pouch 370 may present a flat and clean surface for the user. This makes use of the catheter 320 as parts of the assembly 300 may be placed on the opened pouch 370 as required without them being potentially contaminated with dirt.

After use of the catheter 320, the fluid collection bag 310 may be emptied as described above.

Referring to FIGS. 18 and 19, the catheter 320, fluid collection bag 310, sleeve 330 and reservoir 340, as well as the outlet 360 that is now separated from the bag 310 may be repackaged within the pouch 370. This may be possible in embodiments where the peripheral seal 373 is configured to be re-sealable. Advantageously, any fluid that has leaked from the bag 310 during use or emptying of the bag 310 can be absorbed by the absorbent layer 340 which ensures there are no leaks from the catheter assembly 300 and pouch 370.

To close the pouch 370, the tabs 375, 376 may be brought towards each other, resulting in folding of the pouch about the pouch fold-line 373a and the bringing together of the front wall 371 and rear wall 372. The peripheral seal 373 may then be re-sealed closed, for example by pressing the front wall 371 and rear wall 372 together.

Thus, the pouch 370 may be closed without the user needing to touch the interior surfaces of the pouch 370 which may have become dirty or soiled during use of the catheter 320. In addition, both the tabs 375, 376 may be folded about their respective bases 380 against the front wall 371 of the pouch. This provides an additional mechanical resistance to the re-opening of the peripheral seal 373, reduces the form factor of the pouch 370 and also reduces the likelihood that the tabs 375, 376 are inadvertently separated by contact with another object.

As mentioned above, in this embodiment the catheter 320 and sleeve 330 are in fluid communication with the fluid collection bag 310 via the inlet 353. The configuration of the catheter 320, sleeve 330, inlet 353 and bag 310 is relatively flexible and many different configurations could be employed in embodiments of the invention.

Referring to FIG. 20, in a first example of the configuration for the catheter 320, sleeve 330 and inlet 353 fluid passes directly from the sleeve 330 to the inlet 353 and directly from the catheter 320 to the inlet 353. In this example, a right lateral side 323 of the catheter 320 is attached to a right side 355 of the inlet 353 and there is a gap between a left lateral side 324 of the catheter 320 and the respective left side 356 of the inlet 353. In addition, the distal end 322 of the catheter 320 is within the inlet 353, and the sleeve 330 is sealed around a periphery of the inlet 353. As such, fluid travelling within the sleeve 2 is directed into the inlet 353 directly via the gap between the left lateral side 324 of the catheter 320 and the left side 356 of the inlet 353. In addition, fluid travelling within the catheter 3 passes out the distal end 322 and into the inlet 353 directly. In other examples, the catheter 320 may be positioned differently and may be attached to the left side 356 of the inlet 353, or to a front or rear side.

Referring to FIG. 21, in a second example fluid passes from the catheter 320 into the sleeve 330 and then into the inlet 353. In this example, the right lateral side 323 of the catheter 320 is attached to a left side 334 of the sleeve 330 and a distal end 322 of the catheter 320 is not within the inlet 353. As in the first example, the sleeve 330 is sealed around a periphery of the inlet 353. As such, fluid travelling within the sleeve 302 is directed into the inlet 353 directly and fluid travelling within the catheter 303 passes out the distal end 322 into the sleeve 330 and then from there into the inlet 353. In other examples, the catheter 320 may be positioned differently and may be attached to a left side 335 of the sleeve 330, or indeed to a front or rear side. Furthermore, the distal end 322 of the catheter 320 may be blocked and an outlet (not shown) may be provided in a lateral side of the catheter 320 to allow fluid to pass out into the sleeve 330.

Referring to FIG. 22, in a third example fluid passes from the sleeve 330 into the catheter 320 and then into the inlet 353. The configuration of this example is similar to the first example shown in FIG. 20 and so only the differences are described. In this example, the inlet 353 is sealed between the left side 356 and right side 355 such that the catheter 320 is sealed on all sides. As such, fluid may only pass from the sleeve 330/catheter 320 into the inlet 353 if it is within the catheter 320. The left lateral side 324 of the catheter 320 comprises an oval sleeve intake opening 325 adjacent to the inlet 353 which permits the passage of fluid travelling within the sleeve 303 into the catheter to join fluid travelling within the catheter 302. From here, the fluid 302, 303 passes out the distal end 322 of the catheter 320 and into the inlet 353. Many other configurations may be possible to achieve the same effect, for example, the sleeve 330 may be attached to the outer sides of the catheter 320 itself.

Referring to FIGS. 23-32, a fourth embodiment of a (packaged) catheter assembly 800 is shown. Again, like features are given like reference numerals.

The packaged catheter assembly 800 comprises a pouch 870 containing a fluid collection bag 810, a catheter tube, or “catheter” 820, a sleeve 830, and a fluid reservoir 840 comprising wetting fluid. The catheter 820 has a proximal end 821 for insertion into the body and a distal end 822, and the fluid collection bag 810 is attached to the distal end 822 of the catheter 820 and is arranged to receive fluid from the distal end 822 of the catheter 820, thereby forming a so-called “closed catheter assembly”. In this embodiment, the sleeve 830 and fluid reservoir 840 are also attached to the catheter 820 and so are also considered part of the “closed catheter assembly”.

In this embodiment, the catheter 820 is a male urinary catheter made from a hydrophilic thermoplastic elastomer (TPE). The sleeve 830 of this embodiment is a thermoplastic polyurethane (TPU) or low-density polyethylene (LDPE). Obviously those skilled in the art will be able to select suitable alternative materials.

The sleeve 830 of this embodiment is sized to enclose the catheter 820 along the majority of the length of the catheter 820, and preferably the entire length of the catheter 820. The sleeve 830 comprises a proximal end 831 corresponding to the proximal end 821 of the catheter 820 and a distal end 832 corresponding to the distal end 822 of the catheter 820. The distal end 832 of the sleeve 830 is attached to an inlet 850 of the fluid collection bag 810. The proximal end 831 of the sleeve comprises a reservoir adapter 833.

In this embodiment, the reservoir adapter 833 is generally tubular with a constant wall thickness and comprises a bore through its entire length. Of course, in other embodiments the wall thickness may vary allowing the size/shape of the reservoir adapter to be varied as desired. The reservoir adapter 833 comprises a distal end (not shown) with annular cross-section where it is attached to the proximal end 831 of the sleeve 830. The distal end comprises an internal diameter sized to allow the catheter 820 and wetting fluid to pass through the reservoir adapter 833 as described further below.

The reservoir adapter 833 is configured to releasably engage the fluid reservoir 840 via a jacket as described below. In this embodiment, the reservoir adapter 833 has a size and shape that matches the jacket to allow a push-fit connection between the fluid reservoir 840 and the reservoir adapter 833. This also allows the fluid reservoir 840 to be easily detached from the reservoir adapter 833 by pulling them apart. Of course, in other embodiments the fluid reservoir 840 may be connected in a different way, such as through a bayonet or screw type fit, or through use of a temporary adhesive.

In this embodiment, the fluid reservoir 840 is an elongate rectangular burstable sachet of water. The fluid reservoir 840 is arranged with a first end engaged into the reservoir adapter 833. The reservoir adapter 833 extends around the first end of the fluid reservoir 840 and along the fluid reservoir 840 between 60-20% of the length of the fluid reservoir 840, for example 33%. Consequently, the proximal end of the reservoir adapter 833 comprises a generally elliptical annular cross section to allow an interference fit between the fluid reservoir 840 and reservoir adapter 833.

In this embodiment, the reservoir adapter 833 also comprises a midsection (not shown) between the annular distal end and larger elliptical proximal end enclosing the fluid reservoir 840. The midsection is tapered and has a convex shape to assist the user in gripping the reservoir adapter and using it as a handling element for the catheter 820 and/or sleeve 830. The midsection also acts as a funnel to deliver wetting fluid from the fluid reservoir 840 to the catheter 820/sleeve 830.

In this embodiment, the fluid reservoir 840 is be configured to retain 5 to 50 ml of wetting fluid, for example 82 ml. The fluid reservoir 840 has a height equal to between 20-50% the height of the pouch 870, for example 30%, and a width between 5-25% the width of the pouch 870, for example 15%. The height and width of the pouch 870 are defined as the distance between a base 872a and an upper edge 872d of the pouch, and a left lateral edge 872b and right lateral edge 872c of the pouch 870 respectively and as described in more detail below.

The fluid reservoir 840 also comprises a wetting marker (not shown) configured to allow the user to identify where to activate the fluid reservoir 840 to release wetting fluid. In this embodiment, the wetting marker comprises a logo/region of different colour or contrast from the rest of the fluid reservoir 840. In this embodiment, the wetting marker is positioned in a region of the fluid reservoir 840 that is not enclosed by the reservoir adapter 833, this allows the user to easily identify the wetting marker and activate the fluid reservoir 840. In other embodiments, the wetting marker may provide any suitable visual or tactile indication to assist the user in identifying and activating the fluid reservoir 840 to release wetting fluid.

In this embodiment, the fluid reservoir 840 is configured to release wetting fluid by rupturing upon depression/compression of the fluid reservoir 840, especially at a position corresponding to the wetting marker 844. The fluid reservoir 840 is configured to rupture and create a tear in the fluid reservoir 840 through which wetting fluid is released. In this embodiment, the fluid reservoir 840 is configured to create a tear in locations that are enclosed by the reservoir adapter 833. Thus, the reservoir adapter 833 is therefore configured to receive wetting fluid from the fluid reservoir 840 into the sleeve 830 to wet the catheter 820. To ensure that wetting fluid released from the fluid reservoir 840 is directed into the sleeve 830, the reservoir adapter 833 and/or fluid reservoir 840 may comprise a sealing element to prevent wetting fluid from escaping into the pouch 870, for example an O-ring provided around the end of the reservoir adapter 833 that engages the fluid reservoir 840. Such an O-ring may also facilitate the push-fit engagement of the fluid reservoir 840 and reservoir adapter 833.

In other embodiments, many other types of fluid reservoir would be suitable including different shapes and sizes of reservoir, or non-burstable reservoirs and other external sources of fluid. In this embodiment, the wetting fluid is water and interacts with the hydrophilic surface of the catheter 820 to render it lubricious. In other embodiments, other wetting fluids may be used and they may be polar (e.g. water-based) or non-polar (e.g. oil-based) depending on the catheter's surface properties.

In this embodiment, the fluid reservoir 840 comprises a thermoplastic polyurethane (TPU) or low-density polyethylene (LDPE). In this embodiment, the reservoir adapter 833 comprises a relatively rigid material compared to the sleeve 830, fluid reservoir 840 and catheter 820, for example high-density polyethylene (HDPE), and thus provides a handling element to facilitate movement of the proximal end 831 of the sleeve 830 up and down the catheter 820.

The pouch 870 is formed from a front wall 873 and rear wall 871 of identical shape and size, and a peripheral seal joining the periphery of the walls to form the pouch 870 and containing its contents (i.e. the closed catheter assembly).

The peripheral seal defines a base 872a, a left lateral edge 872b, a right lateral edge 872c and an upper edge 872d of the pouch 870. The left lateral edge 872b and right lateral edge 872c being defined as the left and right sides of the pouch 870 when viewing the pouch 870 with the rear wall 871 behind the front wall, the base 872a at the bottom of the pouch 870 and the upper edge 872d at the top of the pouch 870. The upper edge 872d is linear and the base 872a is outwardly curved, away from the upper edge 872d, and forms a curved bottom of the pouch 870. The peripheral seal thus defines a pouch 870 that is generally rectangular with a curved bottom and suitably has a width between the lateral edges of between 80-160 mm, preferably 100-140 mm, for example about 120 mm, and a height from the lowermost part of the base 872a to the upper edge 872d of between 100 to 250 mm, preferably 120 to 180 mm, more preferably 140 to 160 mm, for example about 150 mm. The base 872a defines the bottom of the pouch 870 in use, and the upper edge 872d the top. However, in other embodiments alternative shapes and sizes could be conceived, such as rectangular, elliptical or circular walls forming correspondingly shaped pouches, or even a tubular pouch or pouch with more than two walls, e.g. a prism or other polygon.

The peripheral seal may be formed from any suitable bonding/sealing methods such as chemical adhesives, chemical bonding, ultrasonic bonding, or mechanical bonding. In alternative embodiments, the pouch 870 may be formed from a single piece of material. The pouch 870 may then be formed by folding this single piece of material along a pouch fold line to form the walls. In addition, in some embodiments any number of folds may be used to form the pouch 870. For example, none of the edges may comprise a fold, one edge may comprise a fold, or two or more of the edges may comprise a fold.

In this embodiment, the pouch 870 is formed from an opaque material and as such provides a discrete package for its contents which can make the user more comfortable in carrying the packaged catheter assembly 800 in day-to-day life. In addition, the exterior of the pouch 870 may have a textured or printed appearance, for example a matte appearance, and feel to reduce the impression that it is a medical device. The front and/or rear wall of the pouch may comprise a wetting marker 882 which provides a visual/tactile guide to help the user locate the fluid reservoir 840 within the closed pouch and activate it to release wetting fluid. In this embodiment, the wetting marker 882 is located on the front wall 873 at the centre of the upper edge 872d of the pouch 870.

In this embodiment, the pouch 870 also comprises an interaction region. The interaction region forms the right side of the pouch 870 and spans between the upper edge 872d and base 872a. The interaction region is used to provide access to the pouch through/near to the right lateral edge 872c. In this embodiment, the interaction region comprises a tear-away region 875. The tear away region 875 is of equal width throughout its length. The tear-away region 875 comprises a tear start 876 at the point the upper edge 872d meets the tear-away region 875, a tear stop 877 at a corresponding point on the base 872a, and a tear line 878 spanning between the tear start 876 and tear stop 877. The tear line 878 comprises a line of weakness in the walls of the pouch 870, which may be formed by any suitable means but in this embodiment is laser etched. The tear start 876 comprises a notch to provide an area of weakness in the peripheral seal. This allows the tear-away region 875 to be separated from the pouch 870 by tearing the walls apart from the tear start 876 to the tear stop 877 along the tear line 878. Thus, the tear is provided beneath the location of the right lateral edge 872c and thus the pouch 870 may be opened provided access to its contents. In other embodiments, the tear stop 877 may comprise a small aperture in the walls to prevent further tearing of the pouch 870.

The tear-away region 875 comprises a circular aperture 879 sized to allow a finger to pass through and grip the tear-away region 875. The circular aperture 879 is positioned in a corner where the upper edge 872d and right lateral edge 872c meet. To ensure the pouch 870 is fully sealed, the front wall 873 and rear wall 871 are sealed to one another over the entire tear-away region 875. In other embodiments, other seals such as a zip-lock arrangement, or hook and hook, hook and loop (VELCRO®) or the like may be used to provide access to the pouch (or to reseal the pouch after opening), and of course, the sizes and shapes set out above are exemplary.

In this embodiment, the catheter 820 is arranged within the pouch 870 in a curved, generally elliptical, curled/coiled configuration. The distal end 822 of the catheter 820 is arranged adjacent to the inner left lateral edge 872b near the base 172a of the pouch 870. The catheter 820 extends from a position where the proximal end 821 of the catheter 820 is adjacent the right lateral edge 872c down the inside of the pouch 870 into a first turn adjacent the base 872a of the pouch and then extends back up the pouch 870 adjacent to the left lateral edge 872b towards the upper edge 872d, into a second turn adjacent the upper edge 872d and then extends back down the pouch adjacent to the right lateral edge 872c towards the base 872a. The catheter 820 then comprises a third turn adjacent the base 872a where the distal end 822 of the catheter 820 connects to the fluid collection bag 810.

In this embodiment, the generally elliptical shape of the catheter 820 helps to maintain the contents of the pouch 870 in a stable position before use and also protects the catheter 820 from damage, for example kinking, due to mechanical stresses imposed on the pouch 870. Of course, the catheter 820 straightens as it is pulled out of the pouch, as described below.

In this embodiment, the fluid collection bag 810 is provided in a stowed configuration, which in this embodiment is a folded configuration, within the pouch 870 and is retained in the folded configuration by the walls of the pouch 870 which provide compressive pressure to the folded fluid collection bag 810. In other embodiments, the fluid collection bag 810 may be retained in the folded configuration by any other suitable means, such as an elastic band, paper band or closure. The folded fluid collection bag 810 is generally rectangular in shape and is arranged within the pouch 870 just below the upper edge 872d and with an axis of the rectangular folded fluid collection bag 810 approximately parallel to the upper edge 872d.

The fluid collection bag 810 comprises an inlet 850 to receive the distal end 822 of the catheter 820 adjacent to the left lateral edge 872b of the pouch 870, and the opposite end of the bag 810 is positioned adjacent to the right lateral edge 872c of the pouch 870. The fluid collection bag 810 is therefore arranged adjacent to the opening formed in the pouch 870 by the tear-away region 875 as described further below and spans between the left lateral edge 872b and right lateral edge 872c.

In this embodiment, the sleeve 830 is arranged around the catheter 820 and covering the majority of the length of the catheter 820. The distal end 832 of the sleeve 830 is attached to the inlet 850 of the fluid collection bag 810, and as described above, the proximal end 831 of the sleeve 830 is attached to the reservoir adapter 833. The reservoir adapter 833 is positioned at the proximal end 821 of the catheter 820 and adjacent the right lateral edge 872c of the pouch 870. The fluid reservoir 840 extends adjacent the inner upper edge 872d from the inner left lateral edge 872b to the inner right lateral edge 872c of the pouch 870. The fluid reservoir of this embodiment is attached to a wall of the pouch, e.g. by adhesive, to prevent it being accidentally removed from the pouch. The fluid reservoir 840 can therefore be activated by pressing the walls of the sealed closed pouch 870 together adjacent the upper edge 872d. Of course, in other embodiments, the catheter 820, fluid collection bag, sleeve 830 with reservoir adapter 833 and fluid reservoir 840 may be arranged differently within the pouch 870.

In this embodiment, the pouch 870 comprises a reservoir jacket 842 configured to enclose the fluid reservoir 840. The reservoir jacket 842 comprises an impermeable material, for example, silicon, a thermoplastic polyurethane (TPU) or low-density polyethylene (LDPE). The reservoir jacket 842 is rectangular and spans across the width of the pouch 870 adjacent to the upper edge of the pouch 870. The reservoir jacket 842 further comprises a wetting aperture 843 arranged at a position adjacent to the opening formed in the pouch 870 by actuation of the interaction region. The wetting aperture is configured to receive an inserter tip 839 of the reservoir adapter 833 and to direct wetting fluid from the reservoir jacket 842 and/or fluid reservoir 840 into the sleeve 830 and onto the catheter 820. In this embodiment, the wetting aperture is resiliently deformed by insertion of the inserter tip 839 therein. Consequently, the wetting aperture provides a fluid tight seal around the inserter tip 839. In other embodiments, the wetting aperture may fit to the reservoir adapter 833 through a different type of fitting, such as a screw or bayonet fitting. This ensures that wetting fluid released by the fluid reservoir 840 is directed into the sleeve 830. Of course, in other embodiments, the reservoir jacket 842 may have a different shape and size and the wetting aperture may be in a different position. Alternatively, the reservoir jacket 842 may couple to the reservoir adapter 833 via a different method, such as being inserted into the reservoir adapter 833.

Furthermore, as the reservoir jacket 842 comprises the wetting aperture, pressure applied to the fluid reservoir 840 to rupture it does not also rupture the reservoir jacket 842, as any increase in pressure within the reservoir jacket 842 is relieved through the wetting aperture 843. Thus, the reservoir jacket 842 remains impermeable to wetting fluid and can direct it into the sleeve 830 irrespective of the location in which the fluid reservoir 840 ruptures.

In this embodiment, the reservoir jacket 842 is attached to the inside of the pouch 870, for example by welding or gluing of the reservoir jacket 842 to the pouch 870. This ensures that the reservoir jacket 842 and fluid reservoir 840 are not removed from the pouch 870 unnecessarily or accidentally, which makes the packaged catheter assembly 800 more convenient to use.

Referring to FIGS. 27 to 29, the fluid collection bag 810 comprises a front panel 811, a rear panel of identical shape and size (not shown), and a peripheral bond joining the periphery of the panels to form the bag 810. The peripheral bond defines a base 812, a right lateral edge 813, a left lateral edge 814 and an upper edge 815. The right lateral edge 813 and left lateral edge 814 being defined as the right and left sides of the bag 810 when viewing the bag 810 with the rear panel behind the front panel 811, the base 812 at the bottom of the bag 810 and the upper edge 815 at the top of the bag 810. The peripheral bond thus defines a bag 810 that, when unfolded, is generally rectangular and suitably has a width between the lateral edges of at least 10, 12, 15 or 20 cm. The bag may have a width no more than 25, 20, 15, 12 or 10 cm. Preferably, the width is between 10 and 20 cm, most preferably between 10 and 15 cm, for example 12 cm. A height from the base 812 to the upper edge 815 may be at least 25, 30, 35 or 40 cm. The height of the bag may be no more than 45, 40, 35 or 30 cm. The height may be between 25 and 40 cm, 28 and 34 cm, or most preferably 29 and 31 cm, for example 30 cm. The bag may be configured to hold at least 500, 700, or 1000 ml of fluid, and may hold no more than 1000 ml. For example, it may hold at least 700 ml of fluid. The base 812 defines the bottom of the bag 810 in use, and the upper edge 815 the top. However, in other embodiments alternative shapes and sizes could be conceived, such as elliptical or circular panels forming correspondingly shaped bags, or even a tubular bag or bag with more than two panels, e.g. a prism or other polygon.

The front panel 811 and rear panel are both substantially formed from an opaque material. In this embodiment, 70% of the fluid collection bag 810 is formed from an opaque material. The remaining 30% of the fluid collection bag 810 is located proximal to the left lateral edge 814 and spans between the upper edge 815 and the base 812.

In this embodiment, the panels comprise a flexible plastics material, for example polypropylene (PP), polyethylene terephthalate (PET), low density polyethylene (LDPE), metalized polyester (MET PET), orientated polypropylene (OPP) or polyvinyl chloride (PVC).

In this embodiment, the inlet 850 is positioned at the upper edge 815 the peripheral bond. The inlet 850 is located at the end of a spout 850a. The spout 850a extends from the upper edge 815 of the fluid collection bag 810 equidistant between the corner where the upper edge 815 and left latera ledge 814 meet and the corner where the upper edge 815 and right lateral edge 813 meet. The spout 850a has a length, i.e. the distance from where the spout 850a joins the upper edge 815 to the inlet 850, of about 8 cm.

In this embodiment, the fluid collection bag 810 comprises a hand strap 817. The hand strap 817 is arranged adjacent to the inlet 850 on a corner of the fluid collection bag 810 where the upper edge 815 and right lateral edge 813 meet. The hand strap 817 is joined to the fluid collection bag 810 by a weld. Thus, the hand strap 817 can be joined to the fluid collection bag 810 simultaneously with the formation of the peripheral bond.

The hand strap 817 provides for a convenient and easy way of holding the fluid collection bag 810, where the hand strap 817 can be temporarily worn around the user's wrist or be easily held. Moreover, the hand strap 817 allows the fluid collection bag 810 to be easily manipulated by the user during use, meaning that the fluid collection bag 810 can be easily handled and manoeuvred by the user as is necessary. Further, the dimensions of the hand strap (as described below) means that the fluid collection bag 810 can be hanged hung from any suitable article, such as a wheelchair handle or bed post, when the catheter assembly is in use.

The hand strap 817 is arranged at the opposite end of the fluid collection bag 810 to an outlet valve 890a (described further below) configured to control release of fluid out of the fluid collection bag 810 in use. This facilitates the flow of fluid within the fluid collection bag 810, including the draining of liquid, toward the outlet valve 890a and away from the inlet 850, when in use.

The hand strap 817 is formed of an opaque material, such as polyethylene terephthalate, polypropylene or low-density polyethylene.

The hand strap 817 has a length (measured from the point at which it is joined to the bag 810 to its furthest point) of 15 cm and a width of 1 cm. The width of the hand strap 817 is constant along its entire length. In this embodiment, the length of the hand strap 817 is fixed, i.e. not adjustable.

The hand strap 817 is formed of a single, continuous loop of material which is joined at two ends to the fluid collection bag 810. The hand strap 817 is joined to the fluid collection bag 810 at two positions on the fluid collection bag 810. In the present embodiment, each of these positions are substantially the same and are each located on the corner where the upper edge 815 and right lateral edge 813 meet. (In this embodiment the distance from one end of the strap to the opposite end, is 30 cm, i.e. 2×15 cm as both ends are attached to the same point.)

In this embodiment, the catheter tube has a length of 35 cm and a width defined as CH14 by the French System, i.e. about 0.5 cm. The catheter is formed of polyolefin material comprising polyethylene and polypropylene.

Referring to FIGS. 23 to 29, the fluid collection bag 810 comprises an outlet valve 890a. The outlet valve 890a is configured to control release of fluid out of the fluid collection bag 810 in use.

The outlet valve 890a is arranged on the front panel 811 of the fluid collection bag 810. The outlet valve 890a is arranged proximal to the base 812, off-centre toward the right lateral edge 813.

The outlet valve 890a comprises a valve base 891a attached to the front panel 811 of the fluid collection bag 810 by a weld. In the present embodiment, the valve base 891a is trapezoidal. The valve base has a height of 8 cm and a width, at its widest part, of 10 cm. The valve base 891a is formed of a material more rigid than the material from which the fluid collection bag 810 is formed. In the present embodiment, the valve base 891a is formed of polypropylene.

The valve base 891a comprises a base opening (not shown) for receiving a liquid output from the fluid collection bag 810. The base opening is an aperture of any suitable size and shape but is preferably a circular aperture having a diameter of at least 0.5 cm.

The outlet valve 890a further comprises an outlet element 893 configured to be laterally rotatable relative to the valve base 891a from a closed configuration to an open configuration. The outlet element 893 is rotatable about a vertical axis of rotation when in use. This means that the fluid collection bag 810, comprising an upper edge, left lateral edge, right lateral edge and base, comprises a vertical axis parallel to the left and right lateral edges, and it is about this axis which the outlet element 893 is rotatable. The outlet element 893 is housed in a housing 898 (best seen in FIG. 29) which is formed integrally with the valve base 891a and sized to receive the outlet element 893 within it, so as to be rotatable between the open and closed configurations.

The outlet element 893 is rotatable such that the outlet element 893 moves through an arc of rotation. At a first end of the arc of rotation, the outlet element 893 is in the closed configuration, and at a second end of the arc of rotation, the outlet element 893 is in the open configuration. Moreover, the outlet element 893 is rotatable about an axis parallel to the plane of the front panel 811 of the fluid collection bag 810 to which the valve base 891a is attached.

The outlet element 893 is has a hollow tubular form with an internal cavity/bore of approximately 1 cm and comprises a circular aperture 897 having a diameter of 1 cm. The aperture 897 opens into, and is hence in fluid communication with the internal cavity of the outlet element 893. The aperture 897 is positioned in an upper portion of the outlet element 893. When the outlet element 893 is in the closed configuration, the aperture 897 is positioned facing outwardly relative to the fluid collection bag 810, facing away from the valve base 891a. In this configuration, the aperture 897 is not aligned with the base opening. Moreover, in the closed configuration, the base opening is aligned with a solid portion of the outlet element 893, therefore, flow of liquid from the fluid collection bag 810 through the base opening is prevented.

When the outlet element 893 is in the open configuration, the aperture 897 is positioned facing inwardly relative to the fluid collection bag 810, facing toward the valve base 891a. In this configuration, the base opening and aperture 897 are in fluid communication. As shown in FIG. 28, liquid 899 within the fluid collection bag 810 can therefore drain from the bag 810 via the base opening, aperture 897 and downwardly through the outlet element and through a connector portion 895b (further described below) and then into a receptacle or toilet or similar.

The connector portion 895b is a hollow cylindrical bore extending from the outlet element such that fluid entering the outlet element 893 via the aperture 897 flows downwardly, through the outlet element 893 and into, through the connector portion 895b and out of a connector portion outlet 895a located at a distal end of the connector portion 895b. The connector portion outlet 895a has a diameter of 1 cm. The connector portion 895b and outlet element 893 each have an inner diameter of 1 cm.

The outlet element 893 comprises a handle 894 arranged between the housing 898 and the connector portion 895b. The handle 894 and connector portion 895b are integrally formed with the outlet element 893 and are formed as a single moulding. The handle 894 is arranged at approximately 90 degrees anti-clockwise relative to the position of the aperture 897. This means that when the outlet element 893 is in the closed position, the handle 894 is arranged against the trapezoidal section of the valve base 891a. Lateral rotation of the outlet element 893 using the handle 894 by 880 degrees moves the handle 894 from a first position closer toward the right lateral edge 813 of the fluid collection bag 810 to a second position closer toward the left lateral edge 814 of the fluid collection bag 810, i.e. to the open position. Simultaneously, this rotates the outlet element 180 degrees such that the aperture is rotated from a position where the aperture 897 is facing away from the valve base 891a and the aperture 897 is not aligned with the base opening, to a position where the aperture is facing toward the valve base 891a and the aperture 897 is in fluid communication with the valve base 891a.

The handle 894 is inhibited from rotating more than 180 degrees clockwise or anti-clockwise due to obstruction by the valve base 891a.

The handle 894 is planar such that when the outlet element 893 is in the open or closed configuration, the handle 894 lies flat against the valve base 891a and only minimally protrudes away from the fluid collection bag 810.

The handle 894 is operable to rotate the outlet element 893 between the closed configuration and the open configuration. The handle 894 is rotatable from a position closer to the right lateral side 813 to a position closer to the left lateral side 814. Such rotation moves the outlet element 893 from the closed configuration to the open configuration.

The handle 894 has a length of 8 cm and a width of 4 cm. The handle 894 is sized to allow a user to easily grip it between at least their thumb and index finger, in particular for users with limited dexterity. The handle 894 is elliptical in shape.

The handle 894 comprises a finger-hole 894a. The finger-hole 894a is sized to allow at least one of the user's fingers to pass through it to facilitate rotation of the handle 894.

The handle 894 extends above the outlet element 893 and valve base 891a to facilitate gripping by the user. When in the closed or open configuration, the handle extends laterally further than the valve base 891a.

The connector portion 895b is configured for use with tubing (not shown) having a diameter marginally smaller than that of the connector portion 895b, such that, on insertion of the tubing onto a distal end of the connector portion 895b, over the connector outlet 895a, in use, the tubing attaches fluid-tight to the connector portion 895b. The annular flange 896 arranged proximal to the connector outlet 895a engages with the tubing.

The valve base 891a, including the housing 898, and outlet element 893, including the connector portion 895b are each formed of a material which is more rigid than the relatively flexible material from which the fluid collection bag is formed. In the present embodiment, the valve base 891a, and outlet element 893 are each formed of polypropylene.

The outlet element 893 protrudes away from the valve base 891a by 1.5 cm.

The flange 896 protrudes away from the housing 898 by 0.3 cm.

Each component of the outlet valve 890a is formed of opaque materials.

Referring to FIGS. 23 to 26, in this embodiment, the packaged catheter assembly 800 is provided with the folded fluid collection bag 810, catheter 820, sleeve 830 and fluid reservoir 840 all contained with the pouch 870 as mentioned above. To wet the catheter 820 so that it is ready for use, the user may release the wetting fluid from the fluid reservoir 840 into the sleeve 830. This may be done by any suitable means, but preferably by applying pressure to the outside of the pouch 870 at a region corresponding to the position of the fluid reservoir 840 by pressing the wetting marker 882.

As described above, in this embodiment, the fluid reservoir 840 is configured to rupture under external pressure and create a tear in the fluid reservoir 840 at a position that is enclosed by the reservoir adapter 833. As such, the wetting fluid is released into the reservoir adapter 833 where it is able to flow through the bore of the reservoir adapter 833 along the length of the catheter 820 between the catheter 820 and sleeve 830.

Referring to FIGS. 24 and 25, in this embodiment, the user may then access the catheter 820 by creating an opening in the pouch 870 using the interaction region. The aperture 879 is grasped in one hand, and the pouch 870 below the tear line 878 in another, and the tear-away region 875 is torn from the tear start 876 to the tear stop 877 along the tear line 878 in a direction corresponding to arrow A in FIG. 24. Thus, the pouch 870 is torn between the upper edge 872d and base 872a at a location below the right lateral edge 872c, as such, an opening is formed in the pouch 870 that may be used to access its contents. In this embodiment, the tear-away region 875 is fully separated from the pouch 870 to improve access to the contents of the pouch 870. In other embodiments, the tear-away region 875 may not be fully separated from the pouch 870 to reduce the number of separate parts and make the pouch 870 easier to handle. Other embodiments may also feature other ways to access the pouch for example a zip-lock seal.

In this embodiment, the closed catheter assembly (i.e. catheter 820 attached to the sleeve 830, reservoir adapter 833, fluid reservoir 840, and fluid collection bag 810) may then be removed from the pouch 870. The folded fluid collection bag 810 is easily accessible to the user as it is directly accessible via the opening in the pouch 870. To withdraw the closed catheter assembly from the pouch 870 the user pulls the closed catheter assembly laterally out of the pouch 870 in the direction of arrow B in FIG. 25.

In this embodiment, the fluid collection bag 810 may then be unfolded so that it is ready to accept fluid from the catheter 820. The catheter 820 may be handled via the sleeve 830 and/or the reservoir adapter 833.

In this embodiment, the fluid reservoir 840 must be removed from the reservoir adapter 833 to allow the proximal end of 821 of the catheter 820 to be withdrawn from the sleeve 830 and inserted into the body. The fluid reservoir 840 is removed from the reservoir adapter 833 by simply pulling the reservoir adapter 833 out of the pouch and hence away from the fluid reservoir 840 which is retained within the pouch. Now that the fluid reservoir 840 is removed from the reservoir adapter 833, the catheter 820 is able to be passed through the reservoir adapter 833 for use. In other embodiments, the fluid reservoir 840 may be actively disengaged by twisting or otherwise activating the reservoir adapter 833 to release the sleeve 830 for example.

The steps of releasing wetting fluid into the sleeve 830, unfolding the fluid collection bag 810, and disengaging the fluid reservoir 840 as described above may be performed in any order depending on the construction of the packaged catheter assembly and the preference of the user.

The catheter 820 is now ready for use. The sleeve 830 may be used to handle the catheter 820 and the reservoir adapter 833 may be used to progressively introduce the proximal end 821 of the catheter 820 into the urethra until fluid flows through the catheter 820 from the bladder. Fluid flowing through the catheter 820 is collected in the fluid collection bag 810. After use, in this embodiment, as the catheter 820 is withdrawn from the body, the reservoir adapter 833 may be used to cover the length of the catheter 820 with the sleeve 830 and the packaged catheter assembly 800 may then be disposed of.

As mentioned above, in this embodiment the catheter 820 and sleeve 830 are in fluid communication with the fluid collection bag 810 via the inlet 850. The configuration of the catheter 820, sleeve 830, inlet 850 and bag 810 is relatively flexible and many different configurations could be employed in embodiments of the invention.

Referring to FIG. 30, in a first example of the configuration for the catheter 820, sleeve 830 and inlet 850 fluid passes directly from the sleeve 830 to the inlet 850 and directly from the catheter 820 to the inlet 850. In this example, a right lateral side 823 of the catheter 820 is attached to a right side 855 of the inlet 850 and there is a gap between a left lateral side 824 of the catheter 820 and the respective left side 856 of the inlet 850. In addition, the distal end 822 of the catheter 820 is within the inlet 850, and the sleeve 830 is sealed around a periphery of the inlet 850. As such, fluid travelling within the sleeve 2 is directed into the inlet 850 directly via the gap between the left lateral side 824 of the catheter 820 and the left side 856 of the inlet 850. In addition, fluid travelling within the catheter 3 passes out the distal end 822 and into the inlet 850 directly. In other examples, the catheter 820 may be positioned differently and may be attached to the left side 856 of the inlet 850, or to a front or rear side.

Referring to FIG. 31, in a second example fluid passes from the catheter 820 into the sleeve 830 and then into the inlet 850. In this example, the right lateral side 823 of the catheter 820 is attached to a left side 834 of the sleeve 830 and a distal end 822 of the catheter 820 is not within the inlet 850. As in the first example, the sleeve 830 is sealed around a periphery of the inlet 850. As such, fluid travelling within the sleeve 2 is directed into the inlet 850 directly and fluid travelling within the catheter 3 passes out the distal end 822 into the sleeve 830 and then from there into the inlet 850. In other examples, the catheter 820 may be positioned differently and may be attached to a left side 835 of the sleeve 830, or indeed to a front or rear side. Furthermore, the distal end 822 of the catheter 820 may be blocked and an outlet (not shown) may be provided in a lateral side of the catheter 820 to allow fluid to pass out into the sleeve 830.

Referring to FIG. 32, in a third example fluid passes from the sleeve 830 into the catheter 820 and then into the inlet 850. The configuration of this example is similar to the first example shown in FIG. 30 and so only the differences are described. In this example, the inlet 850 is sealed between the left side 856 and right side 855 such that the catheter 820 is sealed on all sides. As such, fluid may only pass from the sleeve 830/catheter 820 into the inlet 850 if it is within the catheter 820. The left lateral side 824 of the catheter 820 comprises an oval sleeve intake opening 825 adjacent to the inlet 850 which permits the passage of fluid travelling within the sleeve 3 into the catheter to join fluid travelling within the catheter 2. From here, the fluid 2, 3 passes out the distal end 822 of the catheter 820 and into the inlet 850. Many other configurations may be possible to achieve the same effect, for example, the sleeve 830 may be attached to the outer sides of the catheter 820 itself.

Before use, providing the user does not want the fluid collection bag 810 to drain from the outlet valve 890a during urination, the user must ensure that the outlet valve 890a is in a closed configuration. In this configuration, the handle 894 is arranged closer to the right lateral edge 813 of the fluid collection bag 810 and the aperture 897 therefore positioned facing away from the valve base 891a such that the base opening is adjacent to a solid portion of the outlet element 893. After urination, when the user wishes to drain the fluid collection bag 810, the user grips the handle 894, optionally using the finger-hole 894a, and rotates the handle 894 approximately 880 degrees toward the left lateral edge 814 of the fluid collection bag 810. Simultaneously, the outlet element 893 is rotated. This brings into alignment the aperture 897 and the base opening. As such, fluid from the fluid collection bag 810 can drain through the base opening in the valve base 891a, through the aperture 897, into the outlet element 893 and downwardly through the connector portion 895b and out of the outlet 895a.

Referring to FIGS. 33-42, a fifth embodiment of a packaged catheter assembly 1500 is shown. In the Figures, as is conventional, dashed lines indicate hidden features.

The packaged catheter assembly 1500 comprises a catheter assembly contained within a pouch 1570. The catheter assembly comprises a fluid collection bag 1510, a catheter 1520, a sleeve 1530 and a fluid reservoir 1540. The catheter 1520 has a proximal end 1521 for insertion into the body and a distal end 1522, and the fluid collection bag 1510 is arranged to receive fluid from the distal end 1522 of the catheter 1520, as such the catheter assembly is a “closed catheter assembly”.

In this embodiment, the catheter 1520 is a male urinary catheter made from a hydrophilic thermoplastic elastomer (TPE). The sleeve 1530 of this embodiment is a thermoplastic polyurethane (TPU) or low-density polyethylene (LDPE). Obviously those skilled in the art will be able to select suitable alternative examples.

The pouch 1570 is formed from a front wall 1571a and rear wall 1571b of identical shape and size, and a peripheral seal joining the periphery of the walls to form the pouch 1571 and containing its contents (i.e. the catheter assembly).

The peripheral seal defines a base 1572a, a left lateral edge 1572b, a right lateral edge 1572c and an upper edge 1572d of the pouch 1570. The left lateral edge 1572b and right lateral edge 1572c being defined as the left and right sides of the pouch 1500 when viewing the pouch 1570 with the rear wall 1571b behind the front wall 1571a, the base 1572a at the bottom of the pouch 1570 and the upper edge 1572d at the top of the pouch 1570. The peripheral seal thus defines a pouch 1570 that is generally rectangular with rounded corners and suitably has a width between the lateral edges of between 60-110 mm, preferably 90-110 mm, for example about 105 mm, and a height from the base 1572a to the upper edge 1572d of between 100 to 250 mm, preferably 120 to 170 mm, for example about 150 mm. The base 1572a defines the bottom of the pouch 1570 in use, and the upper edge 1572d the top. However, in other embodiments alternative shapes and sizes could be conceived, such as elliptical or circular walls forming correspondingly shaped pouches, or even a tubular pouch or pouch with more than two walls, e.g. a prism or other polygon.

The peripheral seal may be formed from any suitable bonding/sealing methods such as chemical adhesives, chemical bonding, ultrasonic bonding, or mechanical bonding. In alternative embodiments, the pouch 1570 may be formed from a single piece of material. The pouch may then be formed by folding this single piece of material along a pouch fold line to form the walls. In addition, in some embodiments any number of folds may be used to form the pouch 1570. For example, none of the edges may comprise a fold (as in the illustrated example), one edge may comprise a fold, or two or more of the edges may comprise a fold.

In this embodiment, the pouch 1570 is formed from an opaque material and as such provides a discrete package for its contents which can make the user more comfortable in carrying the packaged catheter assembly 1500 in day-to-day life. In addition, the exterior of the pouch 1570 may have a textured or printed appearance, for example a matte appearance, and feel to reduce the impression that it is a medical device.

In this embodiment, the pouch 1570 also comprises an interaction region which is arranged adjacent to the right lateral edge 1572c of the pouch 1570 on the front wall 1571a. The interaction region is used to provide access to the pouch through/near to the right lateral edge 1572c. In this embodiment, the interaction region comprises a rectangular break-away region 1574. The break-away region 1574 comprises a tear start 1576 at the point the upper edge 1572d meets the break-away region 1575, a tear stop 1577 at a corresponding point on the base 1572a, and a tear line 1578 spanning between the tear start 1576 and tear stop 1577. The tear line 1578 comprises a line of weakness in the walls of the pouch 1570 configured to preferentially tear and allow the break-away region to be at least partially separated from the pouch 1570. The tear line 1578 may be formed by any suitable means but in this embodiment is laser etched.

In this embodiment, the tear line 1578 is positioned off-centre with respect to the width of the pouch 1570. The tear line 1578 is positioned 70-90% across the width of the pouch 1570 from the left lateral edge 1572b to the right lateral edge 1572c, for example 80% across. In other embodiments, the tear-away region may have a different shape or size as required by the shape/size of the pouch 1570 or the contents of the pouch 1570. For example, it may be positioned centrally with respect to the width and/or may be arranged in a different orientation such that access is provided through a corner of the pouch 1570 or a different edge of the pouch 1570.

In this embodiment, the tear line 1578 extends across the front wall 1571a only and not the rear wall 1571b. As such, once the tear line 1578 is torn, the break-away region 1574 remains attached to the pouch 1570 by the rear wall 1571b. This makes the pouch 1570 more convenient to use as the break-away region doesn't become a separate item and so the user doesn't need to handle/store/dispose of an extra item once the tear line 1578 is activated. In other embodiments, the break-away region 1574 may be configured to completely separate from the pouch 1570. This could be advantageous to allow easier access to the pouch 1570.

In this embodiment, the break-away region 1574 comprises a pull-ring 1579 arranged adjacent to the tear start 1576. The pull ring 1579 is sized to allow a finger to pass through and grip then pull so as to tear the front wall 1571a along the tear line 1578 and create an opening in the pouch 1570. Of course, in other embodiments the pull ring 1579 may be replaced by a different kind of easily gripped object, such as a tab.

In this embodiment, the pull-ring 1579 is a different colour from the front wall 1571 of the pouch 1570 to allow the user to more easily recognise it and open the pouch 1570. In addition, an opening marker 1579a in the form of an arrow is present on the front wall 1517a in a position corresponding to the pull-ring 1579 and tear line 1578 and pointing the towards the base 1572a of the pouch 1570. The opening marker 1579a is the same colour as the pull-ring 1579. The user is therefore guided to open the pouch 1570. Of course, in other embodiments, the pull-ring may be a different shade of colour from the pouch, or may be the same colour/shade of colour where discretion is prioritised. In addition, the opening marker may not be present or may have a different form. In some embodiments, it may comprise a region which is a different texture to the pouch for example where the user is visually impaired.

In this embodiment, the fluid reservoir 1540 is configured to retain a wetting fluid and release wetting fluid to activate the surface of the catheter 1520. In this embodiment, the wetting fluid is water and interacts with the hydrophilic surface of the catheter 1520 to render it lubricious. In other embodiments, other wetting fluids may be used and they may be polar (e.g. water-based) or non-polar (e.g. oil-based) depending on the catheter's surface properties. In this embodiment, the fluid reservoir 1540 is configured to retain and release 12 ml of wetting fluid, of course in other embodiments more or less wetting fluid may be required.

In this embodiment, the fluid reservoir 1540 comprises a burstable rectangular sachet 1541 configured to retain 5 to 50 ml of wetting fluid, for example 12 ml. In this embodiment, the fluid reservoir also comprises a jacket in the form of a housing 1542 that surrounds the sachet 1541. The housing 1542 comprises a flexible and fluid impermeable material such as soft silicone. In other embodiments, the housing 1542 may comprise a thermoplastic polyurethane (TPU) or low-density polyethylene (LDPE). The housing 1542 is configured to enclose the sachet 1415 on all sides and is tight fitting on all sides, as such the housing 1542 is also generally rectangular in shape but with rounded edges.

In this embodiment, the housing 1542 is push-fit into a wetting tube 1543 at one end that extends away from the fluid reservoir 1540, of course in other embodiments screw fittings or seals such as a weld may join the housing 1542 to the wetting tube 1543. In this embodiment, the housing 1542 has an aspect ratio (length:width:height) of 5:2:2 and as such is elongate. The wetting tube 1543 connects to a middle of a face of the housing 1542 that is perpendicular to the length of the housing 1542. In this embodiment, the wetting tube 1543 extends in a direction parallel to the length of the housing 1542 and is approximately one third the length of the housing 1542 with a circular cross-section and a diameter approximately half the width and/or height of the housing 1542. The face of the housing 1542 on the opposite side from the wetting tube 1543 is rounded to the extent that it forms a hemispherical end of the housing 1542.

The user can release wetting fluid held by the fluid reservoir 1540 by compressing the fluid reservoir 1540, for example by pinching the housing 1452 between their fingers. To facilitate the user in activating the fluid reservoir 1540, the housing 1542 comprises a wetting marker 1544. The wetting marker 1544 comprises a region of the housing 1542 that is a different colour or texture to the rest of the housing 1542 to allow the user to easily identify the position of the sachet 1451 and where to press to activate it and release wetting fluid. In other embodiments, the fluid reservoir 1540 may be configured to release wetting fluid through a different means, for example by twisting or bursting the fluid reservoir in a different way.

The sleeve 1530 of this embodiment is sized to enclose the catheter 1520 along the majority of the length of the catheter 1520, and preferably the entire length of the catheter 1520. The sleeve 1530 comprises a proximal end 1531 corresponding to the proximal end 1521 of the catheter 1520 and a distal end 1532 corresponding to the distal end 1522 of the catheter 1520. The distal end 1532 of the sleeve 1530 is attached to a valve assembly 1550 as described below. The proximal end 1531 of the sleeve is configured to receive and engage the wetting tube 1543 such that wetting fluid released by the fluid reservoir 1540 is directed into the sleeve 1530 as described further below.

In this embodiment, the proximal end 1531 of the sleeve 1530 comprises a reservoir adapter 1533 configured to retain the fluid reservoir 1540 at the proximal end 1531 of the sleeve 1530. In this embodiment, the reservoir adapter 1533 achieves this by retaining the wetting tube 1543 and housing 1542 in engagement with the sleeve 1530. The reservoir adapter 1533 is fitted around the sleeve 1530 and wetting tube 1543 and comprises an O-ring with an inner diameter that is slightly less than the outer diameter of the wetting tube 1543. Consequently, when the reservoir adapter 1533 is fitted around the wetting tube 1543 and proximal end 1531 of the sleeve 1530, the reservoir adapter 1533 urges the sleeve 1530 against the wetting tube 1543. Thus, a fluid-tight seal is formed between the sleeve 1530 and wetting tube 1543 and the fluid reservoir 1540 is retained in position via the housing 1542.

To ensure tight engagement between the wetting tube 1543 and sleeve 1530, the sleeve 1530 should be formed from a material that is more flexible than both the wetting tube 1543 and reservoir adapter 1533. Preferably, the reservoir adapter 1533 is more flexible than the wetting tube 1543 to ensure the wetting tube 1543 is not compressed when engaged by the reservoir adapter 1533. If the wetting tube 1543 is compressed, this could lead to an unwanted reduction or restriction of wetting fluid flow from the fluid reservoir 1540 to the sleeve 1530. In this embodiment, the reservoir adapter 1533 comprises rubber, however, in other embodiments, a different material may be used such as silicone.

In this embodiment, the reservoir adapter 1533 is configured to release the fluid reservoir 1540 and housing 1542 to allow the proximal end 1521 of the catheter 1520 to pass out of the sleeve 1530 and be used. In this embodiment, the housing 1542 can be released from the reservoir adapter 1533 by being simply pulled out of the sleeve 1530. In other embodiments, specific fittings or tear-lines may be used to allow the housing 1542 to be released from the reservoir adapter 1533. In this embodiment, the wetting tube 1543 is not part of the fluid reservoir 1540 and thus, when the housing 1542 is removed from the sleeve 1530, the wetting tube 1543 remains in the proximal end of the sleeve 1530 to be used as a handling element. In other embodiments, the wetting tube 1543 may be integrally formed with the housing 1542 and may be removed from the sleeve 1530 with the housing 1542.

In this embodiment, the valve assembly 1550 is connected to the distal end 1532 of the sleeve 1530 and the fluid collection bag 1510 and is configured to control the flow of fluids between them. In this embodiment, the valve assembly 1550 comprises two configurations: a first configuration in which the valve assembly 1550 inhibits fluid flow in a direction from the fluid collection bag 1510 towards the catheter 1520 and permits fluid flow in a direction from the catheter 1520 towards the fluid collection bag 1510; and a second configuration in which the valve assembly 1550 permits fluid flow in a direction from the fluid collection bag 1510 towards the catheter 1520 and inhibits fluid flow in a direction from the catheter 1520 towards the fluid collection bag 1510. Thus, the valve assembly 1550 allows the user to both fill and empty the fluid collection bag 1510 via the sleeve 1530 in a controlled manner as described below.

In this embodiment, the valve assembly 1550 comprises a proximal passageway 1551 configured to provide a fluid communication between the valve assembly 1550 and the distal end 1532 of the sleeve 1530 and the catheter 1420, and a distal passageway 1552 configured to provide a fluid connection between the valve assembly 1550 and the fluid collection bag 1510. The connection between the sleeve 1530 and/or catheter 1520 and the proximal passageway 1551 is flexible as described further below. Each passageway 1551, 1552 connects to opposite sides of a side wall of a cylindrical valve housing 1553 and the valve assembly 1550 is configured to control the fluid flow between the passageways 1551, 1552 as shown in FIG. 37 and described below. The valve housing 1553 has a diameter of approximately 1 to 3 cm, for example 2 cm and a length approximately double its diameter. In this embodiment, each passageway 1551, 1552 extends away from the valve housing 1553 by a distance that is not more than the diameter of the valve housing 1553. Each passageway 1551, 1552 is tubular with an internal diameter that is approximately equal to the internal diameter of the catheter 1520, thus each passageway 1551, 1552 has a similar fluid resistance to the catheter 1520 and does not significantly inhibit the flow of fluid into/out of the fluid collection bag 1510.

In this embodiment, one end of the valve housing 1553 is open and is filled by a user operable control which comprises a button 1554 of cylindrical shape. The button 1554 is configured to allow the user to select the first or second configurations of the valve assembly 1550 and may be depressed in an axial direction of the valve housing 1553. In one example, the button 1554 may be in an extended configuration when the valve assembly 1550 is in the first configuration and a depressed configuration when the valve assembly 1550 is in the second configuration. In the extended configuration, the button 1554 extends beyond the open end of the valve housing 1553 and in the depressed configuration, the button 1554 is flush with the open end of the valve housing 1553. The user can switch between the configurations by depressing the button 1554 past the depressed configuration, for example an audible/tactile click may signal that the button 1554 has been sufficiently depressed, for example via a protrusion (not shown) on the inside of the valve housing 1553 that overrides a detent (not shown) on the button 1554 as the button 1554 moves with respect to the valve housing 1553. The user operable control also comprises a biasing element in the form of a spring (not shown) to bias the user operable control and ensure that the button 154 is always returned to the correct extended configuration. Thus, the valve assembly 1550 may be operated using a simple button and can be readily operated by someone with reduced dexterity by simply squeezing the button in their hand. Of course, other configurations of the button 1554 may be readily implemented by the skilled person or indeed a different type of user operable control may be used such as a switch, button, lever, slider, knob. Thus, the valve can be flexibly operated as required by the user.

In this embodiment, the valve housing 1554 comprises a window 1555 located in the side wall of the valve housing 1554 between the passageways 1551, 1552. The window 1555 may be configured to indicate to the user if the valve assembly 1550 is in the first or second configuration as described further below. The window 1555 is elongate with a length parallel to the axis of the valve housing 1554. The window 1555 has a length of approximately half the diameter of the valve housing 1554 and the window has an aspect ratio (length:width) of approximately 3:1. The window is stadium-shaped that is rectangular semicircles replacing two opposite sides.

In this embodiment, the side wall of the button 1554 is a different colour from the valve housing 1553 and a top end of the button 1554 (that is the end that is outside the valve housing 1553) comprises an activation marker 1556. In this embodiment, the activation marker 1556 comprises the words “PUSH” to provide visual guidance on how to operate the valve assembly 1550. In other embodiments, the side wall of the button may be a different shade of colour from the valve housing, and/or the entire button may be a different colour/shade of colour from the valve housing. This can help the user to identify the button 1554 and use the valve assembly 1550.

In addition, the button 1554 is visible through the window 1555 described above. In this embodiment, the parts of the button 1554 that are visible through the window 1555 when the button 1554 is in the extended and depressed configurations respectively are different colours or different shades of colour. This helps the user to identify which configuration the valve assembly 1550 is in. In this embodiment, the window 1555 appears to be the same colour as the button 1554 when the button 1554 is in the extended configuration and is a different colour from the button 1554 when the button 1554 is in the depressed configuration. The user is thereby given a visual indication of the state of the valve assembly 1550.

Inside the valve housing 1553 and between the passageways 1551, 1552, there is a valve body (not shown) that is configured enable the valve assembly 1550 to operate as shown in FIG. 37. The valve body is configured to inhibit fluid flow in a direction from the fluid collection bag 1510 and distal passageway 1552 towards the catheter 1520 and proximal passageway 1551 and permit fluid flow in a direction from the catheter 1520 and proximal passageway 1551 towards the fluid collection bag 1510 and distal passageway 1552 when the button 1554 is in the extended configuration. The valve body is also configured to permit fluid flow in a direction from the fluid collection bag 1510 towards the catheter 1520 and inhibits fluid flow in a direction from the catheter 1520 towards the fluid collection bag 1510 and distal passageway 1552 when the button 1554 is in the depressed configuration. Thus, the valve body enables the valve assembly 1550 to function as required.

The valve body could be any suitable arrangement of fluidic components as would be familiar to the skilled person to enable the valve assembly 1550 to operate as described above. In one example, the valve body comprises two opposing check valves in series: a first check valve to prevent fluid flow in a direction from the catheter 1520 and proximal passageway 1551 towards the fluid collection bag 1510 and distal passageway 1552, and a second check valve to prevent fluid flow in a direction from the fluid collection bag 1510 and distal passageway 1552 towards the catheter 1520/proximal passageway 1551, for example spring loaded in-line check valves could be used for the first and second check valves. The button 1554 is then configured to inhibit the operation of the second check valve in the extended configuration and the first check valve when in the depressed configuration, for example by physically preventing the respective check valve from closing. In a different example, the valve body may comprise two opposing check valves as described above but stacked on top of one another in parallel within the valve housing and coupled to the button 1554. Movement of the button 1554 between the extended and depressed configurations may then select which check valve is in fluid communication with the passageways 1551, 1552 and thus alter the polarity of the valve assembly 1550. Thus, the button 1554 and valve body provide the first and second configurations of the valve assembly 1550. Of course, in other embodiments, a lever, switch or other user operable input may be used to control the valve assembly 1550 and a different type of valve body may be used to achieve the same effect.

In this embodiment, the distal passageway 1552 is connected directly to an access opening 1518 of the fluid collection bag 1510, that is without any intervening components such as tubing. The access opening 1518 is configured to allow fluid to flow into and out of the fluid collection bag 1510 as required. In other embodiments, the distal passageway may be connected indirectly to the access opening of the fluid collection bag 1510a, for example via an intervening section of tubing or other fluid connection device placed between the distal passageway and fluid collection bag.

In this embodiment, the fluid collection bag 1510 comprises a front panel 1511a, a rear panel 1511b of identical shape and size, and a peripheral bond joining the periphery of the panels to form the bag 1510. The peripheral bond defines a base 1512, a right lateral edge 1513, a left lateral edge 1514 and an upper edge 1515. The right lateral edge 1513 and left lateral edge 1514 being defined as the right and left sides of the bag 1510 when viewing the bag 1510 with the rear panel behind the front panel 1511, the base 1512 at the bottom of the bag 1510 and the upper edge 1515 at the top of the bag 1510. The peripheral bond thus defines a bag 1510 that is generally rectangular and preferably square with a width between the lateral edges of between 25 and 40 cm, 30 and 35 cm, or most preferably 33 and 35 cm, and a height from the base 1512 to the upper edge 1515 of between 25 and 40 cm, 30 and 35 cm, or most preferably 33 and 35 cm. The bag 1510 is thus configured to hold at least 700 ml of fluid released from the user through the catheter safely. The base 1512 defines the bottom of the bag 1510 in use, and the upper edge 1515 the top. However, in other embodiments alternative shapes and sizes could be conceived, such as elliptical or circular panels forming correspondingly shaped bags, or even a tubular bag or bag with more than two panels, e.g. a prism or other polygon.

In this embodiment, the fluid collection bag comprises two side panels 1511c and a base panel 1511d. The side panels 1511c are each triangular shaped in the form of elongate isosceles triangles with a height equal to the height of the fluid collection bag 1510. The two equal length sides of each side panel 1511c are bonded to the front and rear panels 1511a, 1511b respectively with one side panel 1511c separating the front and rear panels 1511a, 1511b along the left lateral edge 1514 of the fluid collection bag 1510 and the other separating the front and rear panels 1511a, 1511b along the right lateral edge 1513.

The base panel 1511d that spans between the front panel 1511a and rear panel 1511b along the base 1512 of the peripheral bond and between the base edges of the side panels 1511c. The base panel 1511d is therefore rectangular shaped with a width equal to the width of the fluid collection bag 1510. The base panel 1511d has a length that is equal to the length of the base edge of the side panels 1511c and the base panel 1511d has an aspect ratio (length:width) of approximately 1:4. The base panel 1511d and side panels 1511c thus allow the front panel 1511a and rear panel 1511b to separate along the base 1512 and form a wedge-shaped fluid collection bag 1510.

Consequently, the base panel 1511d and side panels 1511c allow the fluid collection bag 1510 to have a greater internal volume and also to be free-standing when partially filled with fluid as described below.

The base 1512 is formed by two flanges 1512a, 1512b; the rear panel joined to the base panel by the peripheral bond forms a first flange 1512a and the front panel joined to the base panel by the peripheral bond forms the second flange 1512b. The two flanges 1512a, 1512a extend around the left lateral edge 1514, base 1512 and right lateral edge 1513 of the fluid collection bag 1510 where the side panels 1511c and base panel 1511d meets the rear panel 1511b and the front panel 1511a respectively.

In this embodiment, the panels comprise a flexible plastics material, for example polypropylene (PP), polyethylene terephthalate (PET), low density polyethylene (LDPE), metalized polyester (MET PET) orientated polypropylene (OPP), polyvinylpyrrolidone (PVP), or polyvinyl chloride (PVC). Consequently, the panels 1511a, 1511b, 1511c, 1511d may be folded or rolled to reduce the physical size of the fluid collection bag 1510 before, during or after use. In this embodiment, the fluid collection bag 1510 is provided in a stowed configuration before use, which is a folded configuration as described in more detail below.

In this embodiment, the corner of the peripheral bond where the right lateral edge 1514 and base 1512 meet comprises a finger hole 1516 to allow the bag 1510 to be easily handled by a user. The finger hole 1516 is stadium shaped, that is a rectangle with semicircles replacing two opposite sides. The finger hole 1516 comprises two elongate straight edges with a length of one quarter to one third, for example 30%, of the height of the fluid collection bag 1510 and aligned parallel to the right lateral edge 1514. The finger hole 1516 has an aspect ratio (length:width) of approximately 4:1. In other embodiments, the finger hole may comprise a series of ridges, or otherwise easily gripped surface or feature to allow the user to easily handle the bag. In addition, there may be different numbers of finger holes, such as two or more, and they may be placed in different locations on the bag.

In this embodiment, the panels 1511a, 1511b, 1511c, 1511d are formed from an opaque material. This makes the fluid collection bag 1510 more discreet for the user. Of course, in other embodiments, any one or more of the panels may be transparent or translucent.

In this embodiment, the fluid collection bag 1510 further comprises a fill level indicator 1517. The fill level indicator 1517 comprises a region of the front panel 1511a that is formed from a transparent or translucent material configured to allow a user to visualise the amount of liquid within the fluid collection bag 1510. The fill level indicator spans a majority of the height of the fluid collection bag 1510 and in this embodiment spans over 90% of the height of the fluid collection bag 1510. In this embodiment, the fill level indicator 1517 has a width of approximately 30-50% the width of the fluid collection bag 1510, for example 35-40% or most preferably 37%. Thus, the fill level indicator 1517 is a rectangular window that can be used to visualise the contents of the fluid collection bag 1510.

In this embodiment, the fill level indicator 1517 does not contain any fill level markers to allow the fill level of the fluid collection bag 1510 to be quantitatively measured. Of course, in other embodiments, fill level markers may be present for example to let the user identify when the fluid collection bag 1510 is a certain % full, for example the fill level markers may indicate 25%, 50%, and 75% fill levels of the fluid collection bag.

Referring to FIG. 39, in this embodiment, the finger hole 1516, fill level indicator 1517 and access opening 1518 are positioned with respect to one another to enhance the usability of the fluid collection bag 1510.

In this embodiment, the access opening 1518 is positioned in the upper edge 1515 of the fluid collection bag 1510 at a point approximately 15-30% along the upper edge 1515 from the left lateral edge 1514 to the right lateral edge 1513. Thus, the access opening 1518 is positioned at or near an opposite corner of the fluid collection bag 1510 from the finger hole 1516. This allows the user to easily grasp the finger hole 1516 and allow the fluid collection bag 1510 to drain completely through the access opening 1518 while the bag 1510 hangs from the finger hole 1516 and without further manipulation.

In this embodiment, the fill level indicator 1517 spans the majority of the height of the fluid collection bag 1517 and is positioned adjacent to the left lateral edge 1514 of the fluid collection bag 1517. Thus, the fill level indicator 1517 is on the same side of the fluid collection bag 1517 as the access opening 1518 and ensures that during filling and draining the fluid within the fluid collection bag 1510 is visible as soon as it enters the bag and until it leaves the bag. This helps to ensure the fluid collection bag 1510 is completely emptied when draining the bag.

Referring to FIGS. 34 and 35, in this embodiment, the fluid collection bag 1510 is provided in a stowed folded configuration as mentioned above. The fluid collection bag 1510 is folded once at a mid-point along its width and then again at a midpoint along its height thus in the stowed configuration the fluid collection bag 1510 is a square with a width/height half its unfolded width/height. When contained within the pouch 1570, the access opening 1518 is also folded such that the valve assembly 1550 is positioned to overlie the fluid collection bag 1510 and minimise the physical size of the catheter assembly. The valve assembly 1550 is initially provided in the first configuration and as such permits flow of fluids from the catheter 1520 into the fluid collection bag 1510, in addition, the button 1554 is in the extended configuration.

The position and configuration of the catheter 1520 is not particularly limited so long as it is not curved to the extent that damage to the catheter is likely, for example from kinking. In this embodiment, the catheter 1520 is arranged in a curved/coiled configuration, this helps ensure that the catheter 1520 is not excessively curved and minimises risk of damage.

In this embodiment, the fluid reservoir 1540/housing 1542 is positioned adjacent to the base 1572a of the pouch 1570 and beneath the valve assembly 1550. The wetting tube 1543 is therefore positioned adjacent to the left lateral edge 1572b of the pouch 1570. The sleeve 1530 extends from the wetting tube 1543 and completes a hairpin turn before enclosing the proximal end 1521 of the catheter 1520. This takes advantage of the flexible nature of the sleeve 1530 which can be arranged with tight turns/folds to reduce the size of the catheter assembly. This is because even if the sleeve 1530 becomes kinked, kinks in the sleeve 1530 are unlikely to negatively affect the usability of the catheter assembly. It is of course important that any such tight turns/folds are present in a portion of the sleeve 1530 that does not enclose the catheter 1520 as kinks in the catheter 1520 may render it unusable.

Prior to use, the pouch 1570 is provided sealed closed with the contents arranged within it as mentioned above. The contents of the pouch 1570 can be accessed by opening the pouch 1570. The pouch 1570 is opened by grasping the pouch 1570 in one hand and pulling the pull-ring 1579 in the direction indicated by the opening marker 1579a. This breaks the front wall 1571a of the pouch 1570 along the tear line 1578 from the tear start 1576 in the upper edge 1572d to the tear stop in the base 1572a. Thus, an opening is created and the break-away region 1574 is partially removed from the pouch 1570 allowing the catheter assembly to be removed from the pouch 1570 as shown in FIG. 35.

Once the pouch 1570 has been opened, the fluid collection bag 1510, catheter 1520 and sleeve 1530 may be removed from the pouch 1570. To ensure the catheter 1520 is wetted prior to use, the user must activate the fluid reservoir 1540 by squeezing the housing 1542 at the position indicated by the wetting marker 1544. Preferably, the user visualises the wetting fluid leaving the housing 1542 through the wetting tube 1543 and into the sleeve 1530. The wetting fluid enters the sleeve 1530 and flows down the sleeve 1530 wetting the catheter 1520 from its proximal end 1521 to its distal end 1522. This prioritises wetting of the proximal end 1521 and ensures that it is wetted prior to use which is important to reduce the likelihood of injury during use as the proximal end 1521 is the first part of the catheter 1520 to be introduced into the body.

The wetting fluid may then flow through the valve assembly 1550 and into the fluid collection bag 1510 where it is collected.

Before use, the fluid collection bag 1510 may be unfolded to ensure that the collection of liquid within it is not hindered by folds in the fluid collection bag 1510. In addition, the fluid collection bag 1510 may be opened such that the side panels 1551c and base panel 1511d are unfolded and the fluid collection bag 1510 is free-standing on its base. This can be helpful as the user does not need to hold or support the fluid collection bag 1510 and as the access opening 1518 is on the upper edge 1515 of the fluid collection bag 1510, fluid will efficiently fill the bag while it remains standing upright.

The fluid reservoir 1540 and housing 1542 initially block the catheter 1520 from leaving the sleeve 1530 and as such, the user must then remove the housing 1542 from the sleeve by grasping the reservoir adapter 1533 in one hand and pulling the housing 1542 away from it with the other. The reservoir adapter 1533 then separates from the wetting tube 1543 leaving the wetting tube 1543 in place within the sleeve 1530 and restricted by the reservoir adapter 1533. Once the housing 1542 has been removed, the proximal end 1521 of the catheter 1520 may emerge from the sleeve 1530 and be progressively introduced into the body until urine flows therethrough and into the fluid collection bag 1510 via the valve assembly 1550. In this embodiment, the relatively rigid wetting tube 1543 provides a handling element that can be used to guide the proximal end 1521 of the catheter 1520 into the body without having to touch the catheter 1520 directly and risk infection.

Referring to FIG. 39, after use of the catheter 1520, the user may wish to empty the contents of the fluid collection bag 1510. This can be desirable in order to reduce the size and weight of the used bag 1510 and/or to dispose of the bag 1510. To allow the bag 1510 to be emptied, the valve assembly 1550 must be switched into the second configuration by depression of the button 1554 to the depressed configuration. The fluid collection bag 1510 may also be grasped and lifted by the finger hole 1516 to allow gravity to drive fluid flow out of the access opening 1518, through the valve assembly 1550 and out through the catheter 1520/sleeve 1530. The wetting tube 1543 may once again be used to guide the flow of fluid out of the sleeve 1530 to minimise the risk of spillages.

Advantageously, the valve assembly 1550 may be used to selectively start and stop fluid flow through switching between the first and second configurations using the button 1554. This can allow the user to better control draining to minimise leaks/spillage of fluids, for example, they may hang the fluid collection bag 1510 in an elevated position and arrange the sleeve 1530/catheter 1520 into a receptacle such as a drain or toilet before depressing the button 1554 and allowing fluid to drain out of the fluid collection bag 1510.

As mentioned above, in this embodiment the catheter 1520 and sleeve 1530 are in fluid communication with the proximal passageway 1551. The configuration of the catheter 1520, sleeve 5130, and proximal passageway 1551 is relatively flexible and many different configurations could be employed in embodiments of the invention.

Referring to FIG. 40, in a first example of the configuration for the catheter 1520, sleeve 1530 and proximal passageway 1551 fluid passes directly from the sleeve 1530 to the proximal passageway 1551 and directly from the catheter 1520 to the proximal passageway 1551. In this example, a right lateral side 1523 of the catheter 1520 is attached to a right side 1551a of the proximal passageway 1551 and there is a gap between a left lateral side 1524 of the catheter 1520 and the respective left side 1551b of the proximal passageway 1551. In addition, the distal end 1522 of the catheter 1520 is within the proximal passageway 1551, and the sleeve 1530 is sealed around a periphery of the proximal passageway 1551. As such, fluid travelling within the sleeve 2 is directed into the proximal passageway 1551 directly via the gap between the left lateral side 1524 of the catheter 1520 and the left side 1551b of the proximal passageway 1551. In addition, fluid travelling within the catheter 3 passes out the distal end 1522 and into the proximal passageway 1551 directly. In other examples, the catheter 1520 may be positioned differently and may be attached to the left side 1551b of the proximal passageway 1551, or to a front or rear side.

Referring to FIG. 41, in a second example fluid passes from the catheter 1520 into the sleeve 1530 and then into the proximal passageway 1551. In this example, the right lateral side 1523 of the catheter 1520 is attached to a left side 1534 of the sleeve 1530 and a distal end 1522 of the catheter 1520 is not within the proximal passageway 1551. As in the first example, the sleeve 1530 is sealed around a periphery of the proximal passageway 1551. As such, fluid travelling within the sleeve 2 is directed into the proximal passageway 1551 directly and fluid travelling within the catheter 3 passes out the distal end 1522 into the sleeve 1530 and then from there into the proximal passageway 1551. In other examples, the catheter 1520 may be positioned differently and may be attached to a left side 1535 of the sleeve 1530, or indeed to a front or rear side. Furthermore, the distal end 1522 of the catheter 1520 may be blocked and an outlet (not shown) may be provided in a lateral side of the catheter 1520 to allow fluid to pass out into the sleeve 1530.

Referring to FIG. 42, in a third example fluid passes from the sleeve 1530 into the catheter 1520 and then into the proximal passageway 1551. The configuration of this example is similar to the first example shown in FIG. 40 and so only the differences are described. In this example, the proximal passageway 1551 is sealed between the left side 1551b and right side 1551a such that the catheter 1520 is sealed on all sides. As such, fluid may only pass from the sleeve 1530/catheter 1520 into the proximal passageway 1551 if it is within the catheter 1520. The left lateral side 1524 of the catheter 1520 comprises an oval sleeve intake opening 1525 adjacent to the proximal passageway 1551 which permits the passage of fluid travelling within the sleeve 3 into the catheter to join fluid travelling within the catheter 2. From here, the fluid 2, 3 passes out the distal end 1522 of the catheter 1520 and into the proximal passageway 1551. Many other configurations may be possible to achieve the same effect, for example, the sleeve 1530 may be attached to the outer sides of the catheter 1520 itself.

Referring to FIGS. 43 to 45, a variation on the valve assembly 1550 described above is shown. In this example, the valve assembly 1550 is positioned in-line between two sections of tubing rather than directly connected to a fluid collection bag as with the fifth embodiment. This could be advantageous to enable more compact or flexible packaging of the catheter assembly, or to position the valve assembly 1550 in a more convenient place for the user to interact with.

In this example, the valve assembly 1550 is configured such that in the second configuration fluid flow is permitted in both directions, that is from the catheter 1520 towards the fluid collection bag 1510 and vice versa. This retains the core function of the valve assembly 1550 to selectively allow the fluid collection bag 1510 to be drained by switching from the first to second configuration, but does so with a slightly simplified design as one-way flow is not required in the second configuration.

In this example, the valve body could therefore be a single check valve arranged to prevent fluid flow from the fluid collection bag towards the catheter. The button 1554 may be configured to inhibit the check valve from closing when the valve assembly 1550 is in the second configuration thus enabling two-way flow.

As described above, in the first to third and fifth embodiments, the reservoir adapter receives the fluid reservoir/jacket, this allows the reservoir adapter to have a cup-like shape to protect the tip of the catheter as it emerges and also to help guide wetting fluid into the sleeve. In addition, the fluid reservoir of each of these embodiments is removable from the pouch which allows the user to remove the fluid reservoir and catheter from the pouch and then activate the catheter when ready.

Whereas, in the fourth embodiment, the fluid reservoir/jacket receives the reservoir adapter. This is advantageous given the reservoir adapter comprises an inserter tip which must remain clean prior to use. The fluid reservoir/jacket thereby helps keep the tip clean. In addition, the fluid reservoir of the fourth embodiment is retained in the pouch which makes it more convenient to use.

While the embodiments disclosed above may be described as closed catheter assemblies in that fluid is collected in a fluid collection bag, other embodiments may not comprise a fluid collection bag and may be open catheter assemblies in that fluid is not collected during use.

The one or more embodiments are described above by way of example only.

Many variations are possible without departing from the scope of protection afforded by the appended claims.

1. A catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag.

2. The catheter assembly of claim 1 wherein the catheter is a male urinary catheter.

3. The catheter assembly of any preceding claim wherein the catheter is coupled to the fluid collection bag, such that it is configured to collect and retain fluid released from the body through the catheter.

4. The catheter assembly of any preceding claim wherein the catheter assembly is configured such that the fluid reservoir is inhibited from being removed from the sleeve until wetting fluid has been released from the fluid reservoir.

5. The catheter assembly of any preceding claim wherein the sleeve comprises a handling element at one end corresponding to the proximal end of the catheter and the fluid reservoir is disposed on the handling element and releasably engages the handling element, wherein the fluid reservoir is movable axially with respect to the sleeve between a position where it can be removed from the sleeve and a position where it cannot be removed from the sleeve.

6. The catheter assembly of claim 5 wherein the fluid reservoir is configured to release wetting fluid when it is in the position where it can be removed from the sleeve.

7. The catheter assembly of any preceding claim wherein the fluid reservoir is configured to release wetting fluid by pressing the fluid reservoir towards the proximal end of the catheter.

8. The catheter assembly of any preceding claim wherein the sleeve comprises a handling element at one end corresponding to the proximal end of the catheter and the fluid reservoir is disposed on the handling element, wherein the fluid reservoir is configured to release wetting fluid by pressing the fluid reservoir against the handling element and wherein the fluid reservoir is configured to be pierced by the handling element.

9. The catheter assembly of any preceding claim wherein the fluid reservoir is configured to prevent the catheter from being withdrawn from the sleeve.

10. The catheter assembly of claim 9 wherein the fluid reservoir is configured to prevent the catheter from being withdrawn from the sleeve until after the fluid reservoir has released wetting fluid into the sleeve.

11. The catheter assembly of any preceding claim wherein the sleeve comprises a distal end corresponding to the distal end of the catheter and the fluid collection bag comprises an inlet configured to allow fluid to enter the fluid collection bag from the distal end of the sleeve and optionally via the catheter.

12. The catheter assembly of any preceding claim wherein the fluid collection bag comprises an inlet configured to allow fluid to enter the fluid collection bag and the inlet comprises a one-way valve which prevents fluid flow through the inlet from the fluid collection bag to the sleeve and/or catheter.

13. The catheter assembly of claim 11 or 12 wherein the catheter assembly is configured to allow fluid to pass from the distal end of the catheter into the sleeve or from the distal end of the sleeve into the catheter via a sleeve intake opening adjacent to the inlet.

14. The catheter assembly of any preceding claim wherein the sleeve has a diameter no more than four times the diameter of the catheter.

15. A sterile sealed packaged catheter assembly comprising a pouch containing the catheter assembly of any preceding claim.

16. A method of forming a catheter assembly comprising the steps of providing a catheter having a proximal end for insertion into the body and a distal end, arranging a fluid collection bag to receive fluid from the distal end of the catheter, enclosing the catheter along the length of the catheter from the proximal end to the distal end with a sleeve, and arranging a fluid reservoir at the proximal end of the catheter, wherein the fluid reservoir is configured to release wetting fluid into the sleeve to wet the catheter and wherein the sleeve is in fluid communication with the fluid collection bag.

17. A method of wetting a catheter having a proximal end for insertion into the body and a distal end, the method comprising the steps of releasing wetting fluid from a fluid reservoir at the proximal end of the catheter, into a sleeve that encloses the catheter, allowing the wetting fluid to flow through the sleeve from the proximal end to the distal end, and collecting excess wetting fluid in a fluid collection bag configured to receive fluid from a distal end of the catheter and the sleeve.

18. The method of claim 17 wherein the method comprises removing the fluid reservoir from the sleeve.

19. The method of claim 17 or 18 wherein the method comprises removing the fluid reservoir from the sleeve after the release of wetting fluid from the fluid reservoir.

20. The method of any of claims 16 to 19 wherein the catheter assembly is the catheter assembly of any of claims 1 to 15.
